SEQUENCE LISTING (17 sequences)

SEQ ID NO: 1 - protein, 33 aa, Artificial Sequence
  GLP-2-Gly2 Mutation
HGDGSFSDEMNTILDNLAARDFINWLIQTKITD

SEQ ID NO: 2 - protein, 33 aa, Artificial Sequence
  GLP-2 Variant #2
  MISC_FEATURE (10)..(10): Xaa is Nle
  MISC_FEATURE (11)..(11): Xaa is D-Phe
HGEGSFSDEXXTILDNLAARDFINWLIQTKITD

SEQ ID NO: 3 - protein, 33 aa, Artificial Sequence; identical to SEQ ID NO: 2
  GLP-2 Variant #3
  MISC_FEATURE (10)..(10): Xaa is Nle
  MISC_FEATURE (11)..(11): Xaa is D-His

SEQ ID NO: 4 - protein, 33 aa, Artificial Sequence
  GLP-2 Variant #4
  MISC_FEATURE (10)..(10): Xaa is Nle
HGEGSFSDEXNTILDLLAARDFINWLIQTKITD

SEQ ID NO: 5 - protein, 33 aa, Artificial Sequence
  GLP-2 Variant #5
  MISC_FEATURE (10)..(10): Xaa is Nle
HGEGSFSDEXNTILDYLAARDFINWLIQTKITD

SEQ ID NO: 6 - protein, 33 aa, Artificial Sequence; identical to SEQ ID NO: 4
  GLP-2 Variant #6
  MISC_FEATURE (10)..(10): Xaa is Nle

SEQ ID NO: 7 - protein, 33 aa, Artificial Sequence; identical to SEQ ID NO: 5
  GLP-2 Variant #7
  MISC_FEATURE (10)..(10): Xaa is Nle

SEQ ID NO: 8 - protein, 33 aa, Homo sapiens
HADGSFSDEMNTILDNLAARDFINWLIQTKITD

SEQ ID NO: 9 - protein, 33 aa, Artificial Sequence
  GLP-2 Analog
  MISC_FEATURE (2)..(2): Xaa is Gly, Ala or Aib
  MISC_FEATURE (3)..(3): Xaa is Glu, Gln or Asp
  MISC_FEATURE (5)..(5): Xaa is Ser or Thr
  MISC_FEATURE (7)..(7): Xaa is Ser or Thr
  MISC_FEATURE (8)..(8): Xaa is Asp, Glu or Ser
  MISC_FEATURE (9)..(9): Xaa is Glu or Asp
  MISC_FEATURE (10)..(10): Xaa is Met, Val, Leu or Tyr
  MISC_FEATURE (11)..(11): Xaa is Asn, Ser or Ala
  MISC_FEATURE (12)..(12): Xaa is Thr, Ser or Lys
  MISC_FEATURE (13)..(13): Xaa is Ile, Leu, Val, Tyr, Phe or Gln
  MISC_FEATURE (14)..(14): Xaa is Leu or Met
  MISC_FEATURE (15)..(15): Xaa is Asp or Glu
  MISC_FEATURE (16)..(16): Xaa is Asn, Gln, Gly, Ser, Ala, Glu or Lys
  MISC_FEATURE (17)..(17): Xaa is Gln, Lys, Arg, His or Glu
  MISC_FEATURE (19)..(19): Xaa is Ala or Val
  MISC_FEATURE (20)..(20): Xaa is Arg, Lys or His
  MISC_FEATURE (21)..(21): Xaa is Asp, Glu or Leu
  MISC_FEATURE (24)..(24): Xaa is Asn, Ala, Glu or Lys
  MISC_FEATURE (27)..(27): Xaa is Ile, Leu, Val, Glu or Lys
  MISC_FEATURE (28)..(28): Xaa is Gln, Asn, Lys, Ser, Gly-Gly-Pro-Ser-Ser-Gly-Ala-Pro-Pro-Pro-Ser, Lys-Asn-Gly-Gly-Pro-Ser-Ser-Gly-Ala-Pro-Pro-Pro-Ser, or absent
  MISC_FEATURE (29)..(29): Xaa is Thr, Gly-Gly-Pro-Ser-Ser-Gly-Ala-Pro- Pro-Pro-Ser, Lys-Asn-Gly-Gly-Pro-Ser-Ser-Gly-Ala-Pro-Pro-Pro-Ser, or absent
  MISC_FEATURE (30)..(30): Xaa is Lys, Gly-Gly-Pro-Ser-Ser-Gly-Ala-Pro- Pro-Pro-Ser, Lys-Asn-Gly-Gly-Pro-Ser-Ser-Gly-Ala-Pro-Pro-Pro-Ser, or absent
  MISC_FEATURE (31)..(31): Xaa is Ile, Pro, Gly-Gly-Pro-Ser-Ser-Gly-Ala- Pro-Pro-Pro-Ser, Lys-Asn-Gly-Gly-Pro-Ser-Ser-Gly-Ala-Pro-Pro- Pro-Ser, or absent
  MISC_FEATURE (32)..(32): Xaa is Thr, Gly-Gly-Pro-Ser-Ser-Gly-Ala-Pro- Pro-Pro-Ser, Lys-Asn-Gly-Gly-Pro-Ser-Ser-Gly-Ala-Pro-Pro-Pro-Ser, or absent
  MISC_FEATURE (33)..(33): Xaa is Asp, Asn, Gly-Gly-Pro-Ser-Ser-Gly-Ala- Pro-Pro-Pro-Ser, Lys-Asn-Gly-Gly-Pro-Ser-Ser-Gly-Ala-Pro-Pro-Pro- Ser, or absent
HXXGXFXXXXXXXXXXXAXXXFIXWLXXXXXXX

SEQ ID NO: 10 - protein, 33 aa, Artificial Sequence
  GLP-2 Analog
  MISC_FEATURE (2)..(2): Xaa is Gly, Ala or Sar
  MISC_FEATURE (3)..(3): Xaa is Glu or Asp
  MISC_FEATURE (5)..(5): Xaa is Ser or Thr
  MISC_FEATURE (6)..(6): Xaa is Phe or Pro or a conservative substitution
  MISC_FEATURE (7)..(7): Xaa is Ser or Thr
  MISC_FEATURE (8)..(8): Xaa is Asp or Ser or a conservative substitution
  MISC_FEATURE (9)..(9): Xaa is Glu or Asp or a conservative substitution
  MISC_FEATURE (10)..(10): Xaa is Met, Leu, Nle or an oxidatively stable Met-replacement amino acid
  MISC_FEATURE (11)..(11): Xaa is individually selected from the group consisting of Asn, Asp, Glu, Gln, Lys, His, Arg, Ala, Ser, Thr, Pro, Gly, Leu, Ile, Val, Met or Phe
  MISC_FEATURE (12)..(12): Xaa is Thr or Lys or a conservative substitution
  MISC_FEATURE (13)..(13): Xaa is Ile, Glu or Gln or a conservative substitution
  MISC_FEATURE (14)..(14): Xaa is Leu, Met or Nle or a conservative substitution
  MISC_FEATURE (15)..(15): Xaa is Asp or Glu or a conservative substitution
  MISC_FEATURE (16)..(16): Xaa is individually selected from the group consisting of Asn, Asp, Glu, Gln, Lys, His, Arg, Ala, Ser, Thr, Pro, Gly, Leu, Ile, Val, Met or Phe
  MISC_FEATURE (17)..(17): Xaa is Leu or Glu or a conservative substitution
  MISC_FEATURE (18)..(18): Xaa is Ala or Aib or a non-conservative substitution
  MISC_FEATURE (19)..(19): Xaa is Ala or Thr or a conservative substitution
  MISC_FEATURE (20)..(20): Xaa is selected from the group consisting of Asn, Asp, Glu, Gln, His, Arg, Ala, Ser, Thr, Pro, Gly, Leu, Ile, Val, Met or Phe
  MISC_FEATURE (21)..(21): Xaa is Asp or Ile or a conservative substitution
  MISC_FEATURE (24)..(24): Xaa is individually selected from the group consisting of Asn, Asp, Glu, Gln, Lys, His, Arg, Ala, Ser, Thr, Pro, Gly, Leu, Ile, Val, Met or Phe
  MISC_FEATURE (28)..(28): Xaa is individually selected from the group consisting of Asn, Asp, Glu, Gln, Lys, His, Arg, Ala, Ser, Thr, Pro, Gly, Leu, Ile, Val, Met or Phe
  MISC_FEATURE (31)..(31): Xaa is Pro, Ile or deleted
  MISC_FEATURE (32)..(32): Xaa is Thr or deleted
  MISC_FEATURE (33)..(33): Xaa is Asp, Asn or deleted
HXXGXXXXXXXXXXXXXXXXXFIXWLIXTKXXX

SEQ ID NO: 11 - protein, 30 aa, Artificial Sequence
  GLP-2 Analog
  MISC_FEATURE (10)..(10): Xaa is Nle
  MISC_FEATURE (11)..(11): Xaa is D-Thi
HGDGSFSDEXXTILDFLAARDFINWLIQTK

SEQ ID NO: 12 - protein, 30 aa, Artificial Sequence; identical to SEQ ID NO: 11
  GLP-2 Analog
  MISC_FEATURE (10)..(10): Xaa is Nle
  MISC_FEATURE (11)..(11): Xaa is D-Phe

SEQ ID NO: 13 - protein, 33 aa, Artificial Sequence
  GLP-2 Analog
  MISC_FEATURE (10)..(10): Xaa is Nle
  MISC_FEATURE (11)..(11): Xaa is D-Phe
HGDGSFSDEXXTILDLLAARDFINWLIQTKITD

SEQ ID NO: 14 - protein, 33 aa, Artificial Sequence
  GLP-2 Analog
  MISC_FEATURE (10)..(10): Xaa is Nle
  MISC_FEATURE (11)..(11): Xaa is D-Thi
HGDGSFSDEXXTILDLLATRDFINWLIQTKITD

SEQ ID NO: 15 - protein, 33 aa, Artificial Sequence
  GLP-2 Analog
  MISC_FEATURE (10)..(10): Xaa is Nle
  MISC_FEATURE (11)..(11): Xaa is D-Phe
HGDGSFSDEXXTILDFLAARDFINWLIQTKITD

SEQ ID NO: 16 - protein, 33 aa, Artificial Sequence; identical to SEQ ID NO: 15
  GLP-2 Analog
  MISC_FEATURE (10)..(10): Xaa is Nle
  MISC_FEATURE (11)..(11): Xaa is D-Thi

SEQ ID NO: 17 - protein, 33 aa, Artificial Sequence
  GLP-2 Analog
  MISC_FEATURE (2)..(2): Xaa can be any amino acid
  MISC_FEATURE (3)..(3): Xaa can be any amino acid
  MISC_FEATURE (10)..(10): Xaa can be any amino acid
  MISC_FEATURE (11)..(11): Xaa can be any amino acid
  MISC_FEATURE (16)..(16): Xaa can be any amino acid
HXXGSFSDEXXTILDXLAARDFINWLIQTKITD

(19) United States
(12) Patent Application Publication
(10) Pub. No.: US 2020/0254065 A1
(43) Pub. Date: Aug. 13, 2020

(54) LONG-ACTING GLP-2 ANALOGS

(71) Applicant: OPKO Biologics Ltd., Kiryat Gat (IL)

(72) Inventors: Oren Hershkovitz, M.P. Shikmim (IL); Ahuva Bar-Ilan, Rehovot (IL); Vered Lev, Rehovot (IL)

(73) Assignee: OPKO Biologics Ltd., Kiryat Gat (IL)

(21) Appl. No.: 16/787,810

(22) Filed: Feb. 11, 2020

Related U.S. Application Data
(60) Provisional application No. 62/804,201, filed on Feb. 11, 2019.

(51) Int. Cl. A61K 38/26; A61K 47/65; A61K 47/60

(52) U.S. Cl. CPC A61K 38/26; A61K 47/60; A61K 47/65

(57) ABSTRACT

Compositions which include glucagon-like peptide-2 (GLP-2) analogs, GLP-2 analogs with reversible or non-reversible linkers attached to one or more amino acid positions of the GLP-2 analog, and GLP-2 analogs linked to one or more polyethylene glycol polymers (PEG) via reversible or non-reversible linkers are disclosed. Also disclosed are pharmaceutical compositions comprising: the GLP-2 analogs; GLP-2 analogs linked solely to reversible or non-reversible linkers; the reverse PEGylated GLP-2 analogs; and the non-reversibly PEGylated GLP-2 analogs, as well as methods of using the same.

### FIELD OF INVENTION

Compositions which include glucagon-like peptide-2 (GLP-2) analogs, GLP-2 analogs with reversible or non-reversible linkers attached to one or more amino acid positions of the GLP-2 analog, and GLP-2 analogs linked to one or more polyethylene glycol polymers (PEG) via reversible or non-reversible linkers are disclosed. Also disclosed are pharmaceutical compositions comprising: the GLP-2 analogs; GLP-2 analogs linked solely to reversible or non-reversible linkers; the reverse PEGylated GLP-2 analogs; and the non-reversibly PEGylated GLP-2 analogs, as well as methods of using the same.

### BACKGROUND OF THE INVENTION

Glucagon-like peptide-2 (GLP-2) is a 33-amino-acid proglucagon-derived peptide produced by and secreted from enteroendocrine L cells located primarily in the lower gastrointestinal tract. GLP-2 circulates at low basal levels in the fasting period, and plasma levels rise rapidly after food ingestion. Its activity is mediated through the G protein coupled receptor for GLP-2. GLP-2 affects multiple facets of intestinal physiology, foremost among these is the ability to increase small and large intestine weight through stimulation of epithelial cell proliferation and inhibition of apoptosis leading to enlarged crypts and villi and, hence, an enhanced absorptive surface area and increased nutrient assimilation.

The GLP-2 peptide is a product of the proglucagon gene. Proglucagon is expressed mainly in the pancreas and the intestine and to some extent in specific neurons located in the brain. The posttranslational processing of proglucagon is however different in pancreas and intestine. In the pancreas proglucagon is processed mainly to Glucagon Related Pancreatic Polypeptide (GRPP), Glucagon and Major Proglucagon Fragment. In contrast to this, the processing in the intestine results in Glicentin, Glucagon-Like Peptide 1 (GLP-1) and Glucagon-Like Peptide 2 (GLP-2).

GLP-2 is intended for the treatment of short bowel syndrome (SBS), a malabsorption disorder caused by surgical resection, congenital defect, or disease-associated loss of intestinal absorption. SBS is characterized by the inability to maintain protein-energy, fluid, electrolyte, or micronutrient balances. Treatment with GLP-2 had shown significant improvement in wet weight, increases in relative energy, macronutrient and electrolyte absorption. In rodents, GLP-2 showed significant increase in small intestinal mass.

GLP-2 induces significant growth of the small intestinal mucosal epithelium via the stimulation of stem cell proliferation in the crypts and inhibition of apoptosis in the villi (Drucker et al., Proc Natl Acad Sci USA 93:791 1-7916 (1996)). GLP-2 also has growth effects on the colon. Furthermore, GLP-2 inhibits gastric emptying and gastric acid secretion (Wojdemann et al., J Clin Endocrinol etab. 84:2513-2517 (1999)), enhances intestinal barrier function (Benjamin et al., Gut47:1 12-9 (2000)), stimulates intestinal hexose transport via the upregulation of glucose transporters (Cheeseman, Am J Physiol. R1965-71 (1997)), and increases intestinal blood flow (Guan et al., Gastroenterology: 138147 (2003)).

GLP-2 has been shown to prevent weight loss and reduce the severity of epithelial damage in mice with dextran sulfate-induced colitis and been shown to exert therapeutic actions in a wide number of preclinical models of gut injury (Sinclair, Elaine M., and Daniel J. Drucker. “Proglucagon-derived peptides: mechanisms of action and therapeutic potential.” Physiology 20.5 (2005): 357-365). GLP-2 analogs have also been shown to significantly reverse weight loss, reduce interleukin-1 expression, and increase colon length, crypt depth, and both mucosal area and integrity in the colon of mice with acute DS colitis (Drucker, Daniel J., et al. “Human [Gly2] GLP-2 reduces the severity of colonic injury in a murine model of experimental colitis.” American Journal of Physiology-Gastrointestinal and Liver Physiology 276.1 (1999): G79-G91). There is also evidence that GLP-2 may play a role in mucosal healing and maintenance mechanisms in coeliac disease (Caddy, Grant R., et al. “Plasma concentrations of glucagon-like peptide-2 in adult patients with treated and untreated coeliac disease.” European journal of gastroenterology & hepatology 18.2 (2006): 195-202).

GLP-2 has been shown to maintain intestinotrophic activity, such as small bowel growth, pancreatic islet growth, and/or increase in crypt/villus height, in a vertebrate. The effect of GLP-2 on small bowel also manifests as an increase in the height of the crypt plus villus axis. Such activity is referred to herein as an “intestinotrophic” activity. Also detectable in response to GLP-2 is an increase in crypt cell proliferation and/or a decrease in small bowel epithelium apoptosis. These cellular effects are noted most significantly in relation to the jejunum, including the proximal jejunum, distal jejunum, and distal ileum, and are also noted in the distal ileum.

The biological half-life of circulating native GLP-2 is relatively short, approximately 7 minutes in humans, due to extensive renal clearance and rapid degradation by the proteolytic enzyme DPP-1V. Hence, GATTEX®, the only available commercial GLP-2 treatment, differs from GLP-2 natural sequence, in the substitution of alanine (in native GLP-2) for glycine at the second position at the N-terminus (teduglutide). This single amino acid substitution provides certain resistance to in vivo degradation of teduglutide by dipeptidyl protease-IV (DPP-IV) resulting in an extended half-life (See for example WO 97/39031). Nevertheless, SBS treatment with GATTEX® requires a restrictive injection regimen based on daily injections and powdered formulation required to be dissolved before each injection.

One critical disadvantage of GLP-2 peptides and analogs is their very short half-lives in vivo, necessitating infusion or frequent injections. The principal metabolic pathway for GLP-2 clearance is through enzymatic degradation. GLP-2 has been shown to be rapidly degraded through the removal of its two N-terminal amino acids by dipeptidylpeptidase-IV (DPP-IV), which represents a major limitation because it leads to the complete inactivation of the peptide. The in vivo half-life of native GLP-2 is approximately 7 minutes. The in vivo half-life of GATTEX® is approximately 2 to 3 hours.

New conceptual approach termed reversible pegylation was previously described (PCT Publication No. WO 98/05361; Gershonov et al., 2000), for prolonging the half-life of proteins and peptides. According to this technology, prodrugs are prepared by derivatizing the drug with functional groups that are sensitive to pH conditions and removable under natural to basic conditions such as physiological conditions. The derivatization includes a substitution of at least one amino, hydroxyl, mercapto and/or carboxyl groups of the drug molecule with a linker such as 9-fluorenylmethoxycarbonyl (Fmoc) and 2-sulfo-9-fluorenylmethoxycarbonyl (FMS), to which a group of PEG moiety is attached. The link between the PEG moiety and the drug is not direct but rather both residues are linked to different positions of the scaffold FMS or Fmoc structures that are highly sensitive to pH conditions. The present invention relates to GLP-2 derivative in which the half-life of the peptide is prolonged utilizing peptide sequence optimization and the reversible PEGylation technology.

A drug that has a longer half-life, improved efficacy and more convenience is needed not only for SBS patients but also for other indications embodied throughout the present application.

### SUMMARY OF THE INVENTION

In one aspect, disclosed is a compound of the formula: L-GLP-2, wherein, L is a linker group; and GLP-2 is a GLP-2 analog or variant having one or more specific amino acid mutations as compared to wild type GLP-2.

In a related aspect, the linker group in the compound is 2-methoxy-9-fluorenylmethoxycarbonyl (MeOFmoc), 2,5-dioxopyrrolidin-1-yl-3-(2-(3-(2,5-dioxo-2,5-dihydro-1H-pyrrol-1-yl)propanamido)-9H-fluoren-9-yl)propanoate (“NRFmoc”), 9-fluorenylmethoxycarbonyl (Fmoc), MAL-Fmoc, Fmoc-Osu, 2-sulfo-9-fluorenylmethoxycarbonyl (FMS), MAL-FMS, or FMS-Osu.

In a one aspect, disclosed is a compound of compound selected from one of the following formulas:

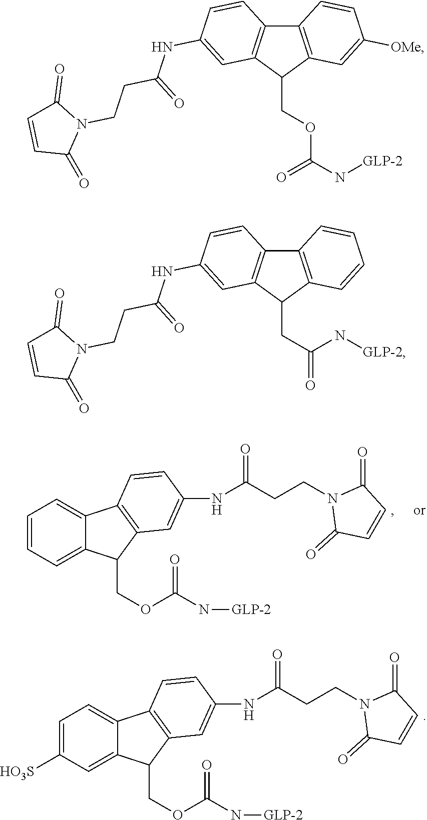

In a one aspect, disclosed is a compound selected from one of the following:

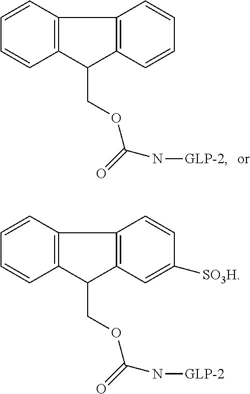

In a related aspect, the linker containing a maleimide group is further reacted with a thiol-containing molecule. In another aspect, the thiol-containing molecule is cysteine or cysteamine. In a related aspect, the reacting with a thiol-containing molecule results in the reduction of the MAL-linker-GLP-2 such as maleimide hydrogenation, and/or the coupling of the thiol-containing molecule to the linker-GLP-2.

In one aspect, disclosed is a compound further comprising the formula X-L-GLP-2, wherein X is selected from a polymeric compound. In a related aspect, X a polyethylene glycol polymer (“PEG”). In a related aspect, the PEG is PEG2, PEG10, PEG20, PEG30, PEG40, or PEG60. In a related aspect, the PEG has a molecular weight in the range of 2,000 to 50,000 Da.

In one aspect, disclosed herein is a GLP-2 analog or variant having an amino acid sequence according the formula: R1-His1-X2-X3-Gly4-Ser5-Phe6-Ser7-Asp8-Glu9-X10-X11-Thr12-Ile13-Leu14-Asp15-X16-Leu17-Ala18-Ala19-Arg20-Asp21-Phe22-Ile23-Asn24-Trp25-Leu26-Ile27-Gln28-Thr29-Lys30-Ile31-Thr32-Asp33-R2, wherein R1 may be OH, COOH, NH₂, CONH₂, or CONHNH₂; X2 may be Ala or Gly; X3 may be Asp or Glu; X10 may be Met or Nle; X11 may be Asn, D-Phe, or D-His; X16 may be Asn, Leu, or Tyr; and R2 may be OH, COOH, NH₂, CONH₂, or CONHNH₂. In a related aspect, the GLP-2 analog or variant has an amino acid sequence according to any one of SEQ ID NO: 1 through SEQ ID NO: 16.

In one aspect, disclosed is a compound of the formula:

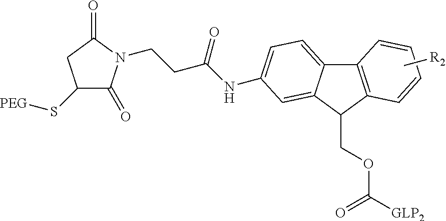

wherein PEG is a polyethylene glycol polymer; R2 is H, O—CH₃, or SO₃H; and GLP2 is a GLP2 analog or variant having one or more specific amino acid mutations as compared to wild type GLP-2. In a related aspect, the GLP-2 analog or variant has an amino acid sequence according to any one of SEQ ID NO: 1 through SEQ ID NO: 16.

In one aspect, disclosed is a pharmaceutical composition comprising any compound disclosed herein, or a salt or derivative thereof, in a mixture with a carrier.

In one aspect, disclosed is a method for the treatment of a bowel disease, small bowel syndrome, inflammatory bowel syndrome, colitis including collagen colitis, radiation colitis, ulcerative colitis chronic radiation enteritis, non-tropical (gluten intolerance) and tropical sprue, Coeliac disease (gluten sensitive enteropathy), damaged tissue after vascular obstruction or trauma, diarrhea e.g. tourist diarrhea and post-infective diarrhea, chronic bowel dysfunction, dehydration, bacteremia, sepsis, anorexia nervosa, damaged tissue after chemotherapy e.g. chemotherapy-induced intestinal mucositis, premature infants incl. intestinal failure in premature infants, preborn infants incl. intestinal failure in preborn infants, schleroderma, gastritis including atrophic gastritis, postantrectomy atrophic gastritis and Helicobacter pylori gastritis, pancreatitis, general septic shock ulcers, enteritis, cul-de-sac, lymphatic obstruction, vascular disease and graft-versus-host, healing after surgical procedures, post radiation atrophy and chemotherapy, weight loss in Parkinson's Disease, intestinal adaptation after surgical procedure, parenteral nutrition-induced mucosal atrophy, e.g. total parenteral nutrition (TPN)-induced mucosal atrophy, and bone-related disorders including osteoporosis, hypercalcemia of malignancy, osteopenia due to bone metastases, periodontal disease, hyperparathyroidism, periarticular erosions in rheumatoid arthritis, Paget's disease, osteodystrophy, myositis ossificans, Bechterew's disease, malignant hypercalcemia, osteolytic lesions produced by bone metastasis, bone loss due to immobilization, bone loss due to sex steroid hormone deficiency, bone abnormalities due to steroid hormone treatment, bone abnormalities caused by cancer therapeutics, osteomalacia, Bechet's disease, osteomalacia, hyperostosis, osteopetrosis, metastatic bone disease, immobilization-induced osteopenia, or glucocorticoid-induced osteoporosis, the method comprising administering a therapeutically or prophylactically effective amount of the compositions disclosed herein.

A method for the treatment of acid-induced intestinal injury, arginine deficiency, autoimmune diseases, bacterial peritonitis, bowel ischemia, bowel trauma, burn-induced intestinal damage, catabolic illness, celiac disease, chemotherapy-associated bacteremia, chemotherapy-induced enteritis, decreased gastrointestinal motility, diabetes, diarrheal diseases, fat malabsorption, febrile neutropenia, food allergies, gastric ulcers, gastrointestinal barrier disorders, gastrointestinal injury, hypoglycemia, idiopathic hypospermia, inflammatory bowel disease, intestinal failure, intestinal insufficiency, irritable bowel syndrome, ischemia, malnutrition, mesenteric ischemia, mucositis, necrotizing enterocolitis, necrotizing pancreatitis, neonatal feeding intolerance, neonatal nutritional insufficiency, NSAID-induced gastrointestinal damage, nutritional insufficiency, obesity, pouchitis, radiation-induced enteritis, radiation-induced injury to the intestines, steatorrhea, stroke, or total parenteral nutrition damage to gastrointestinal tract, the method comprising administering a therapeutically or prophylactically effective amount of the compositions disclosed herein.

A method for increasing the crypts plus villi depth and length in a patient, the method comprising administering a therapeutically or prophylactically effective amount the compositions disclosed herein.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows the pharmacology effect of different GLP-2 Variant #4 conjugates as measured by their respective percent increases of crypts plus villi length over vehicle.

FIG. 2 shows the pharmacology effect of different GLP-2 Variant #4 conjugates as measured by their respective percent increases of crypts plus villi length over vehicle

FIG. 3 shows the dose dependent pharmacology comparison between GLP-2 Variant #4 to a reversible MAL-FMS-V4 conjugate as measured by their respective percent increases of crypts plus villi length over vehicle.

FIG. 4 describes RP-HPLC chromatogram showing the FMS-coupled peptide following cleavage from the resin and following acid treatment.

FIG. 5 describes RP-chromatogram of the purified coupled peptide and the cysteinated FMS-peptide where the cysteine covalently react with the maleimide group to yield Cys-FMS-V4.

FIG. 6 describes MALDI-TOF analysis of the Cys-FMS-V4 having the expected MW of 3335 consisting from MAL-FMS-V4 MW of 4214 g/mol and the 121 g/mol gained from covalent reaction with the cysteine (121 g/mol).

FIG. 7 shows a scheme of the synthesis of the MAL-Fmoc-NHS and MAL-FMS-NHS linkers.

FIG. 8 shows a scheme of the synthesis of the MAL-Fmoc-NHS liner.

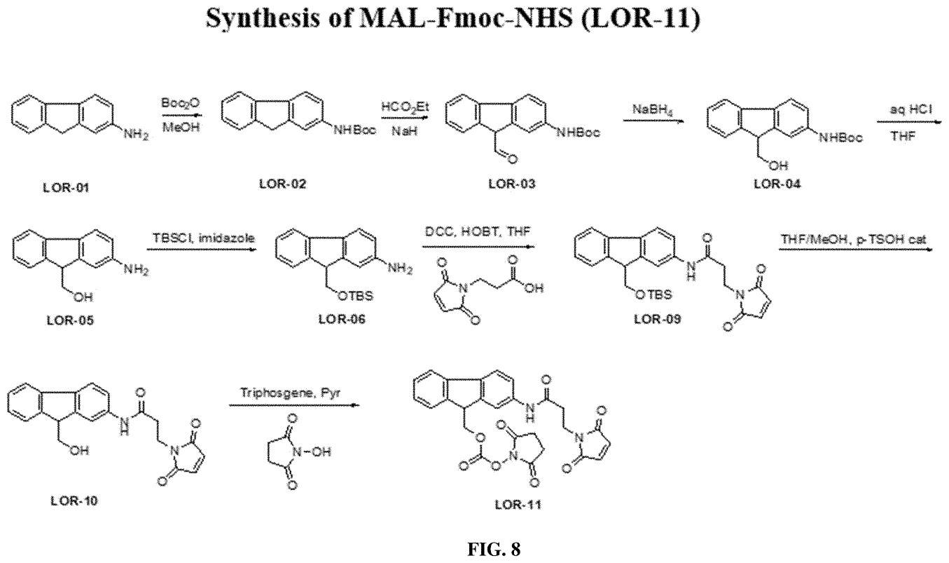

FIG. 9 shows the different homogenous and heterogenous products synthesized: PEG-Linker-(N-terminal)-(GLP-2 Variant) and PEG-Linker-(Lys30)-(GLP Variant).

FIG. 10 shows the structure of Fmoc-Osu-(GLP-2 Variant #4), where the Fmoc-Osu linker attached is considered a mono-functional linker enabling one covalent binding to the peptide.

FIG. 11 shows the structure of FMS-Osu-(GLP-2 Variant #4) where the FMS-Osu linker attached is considered as a mono-functional linker enabling one covalent binding to the peptide

FIG. 12 shows the pharmacology effect of V4 and different V4-conjugates compared to Apraglutide and Glepaglutide as measured by their respective percent increase of small intestine weight over vehicle.

FIG. 13 shows the pharmacology effect of V4 and different V4-conjugates compared to Apraglutide and Glepaglutide as measured by their respective percent increases of crypts plus villi length over vehicle.

FIG. 14 shows the pharmacology effect of V4 and Cys/OSu-FMS-V4 compared to Apraglutide, Glepaglutide, and Gattex as measured by their respective percent increases of crypts plus villi length over vehicle up to 14 days.

FIG. 15 shows the acute and long acting dose dependent pharmacology effect of V4 and Cys-FMS-V4 as measured by their respective percent increase of small intestine weight over vehicle.

FIG. 16 shows the acute and long acting dose dependent pharmacology effect of V4 and Cys-FMS-V4 as measured by their respective percent increases of crypts plus villi length over vehicle.

FIG. 17 shows the PK profile of V4, Cys-FMS-V4 and Apraglutide in rats following a single 2 mg/kg SC injection.

FIG. 18 shows the PK profile of V4 and Cys-FMS-V4 in rats following a single 2 mg/kg IV injection.

FIG. 19 shows the PK model that was used for analysis of the observed PK profile of V4 and Cys-FMS-V4.

FIG. 20 shows a simulation of the time course of V4 and Apraglutide plasma concentrations in rats, based on the PK profile and analysis.

### DETAILED DESCRIPTION OF THE INVENTION

In one embodiment, “amino acid” or “amino acids” is understood to include the 20 naturally occurring amino acids; those amino acids often modified post-translationally in vivo, including, for example, hydroxyproline, phosphoserine and phosphothreonine; and other unusual amino acid including, but not limited to, 2-aminoadipic acid, hydroxylysine, isodesmosine, nor-valine, nor-leucine and ornithine. Throughout the description and claims the conventional one-letter and three-letter codes for natural amino acids are used as well as generally accepted three letter codes for other α-amino acids, such as sarcosin (Sar), norleucine (Nle) and α-aminoisobutyric acid (Aib). In one embodiment, “amino acid” includes both D- and L-amino acids. It is to be understood that other synthetic or modified amino acids can be also be used.

In one embodiment, the terms “analog”, “analogue”, or “variant” are meant to include amino acid sequences comprising peptides with different amino acid sequences from the native sequence, such as the GLP-2 sequence, but with similar or comparable activity.

In another embodiment, the phrases “long acting GLP-2 analog” is used to refer to a GLP-2 analog with specific amino acid mutations as compared to wild type GLP-2; a GLP-2 analog with 9-fluorenylmethoxycarbonyl (Fmoc), a maleimide moiety of Fmoc (MAL-Fmoc), 2-sulfo-9-fluorenylmethoxycarbonyl (FMS), a maleimide moiety of FMS (MAL-FMS), 2-methoxy-9-fluorenylmethoxycarbonyl (MeOFmoc), or 2,5-dioxopyrrolidin-1-yl-3-(2-(3-(2,5-dioxo-2,5-dihydro-1H-pyrrol-1-yl)propanamido)-9H-fluoren-9-yl)propanoate (NRFmoc) attached to one or more amino acid positions of the GLP-2 analog; a reversibly PEGylated GLP-2 analog; or an irreversible PEGylated GPL-2 in the case where the GLP-2 analog is linked to a NRFmoc linker.

The GLP-2 analogues of the present invention have one or more amino acid substitutions, deletions, inversions, or additions compared with native GLP-2 and as defined above. This definition also includes the synonym terms GLP-2 mimetics and/or GLP-2 agonists.

Compounds of the present invention have at least one GLP-2 biological activity, in particular in causing growth of the intestine. This can be assessed in in vivo assays, for example as described in the Examples, in which the mass of the intestine, or a portion thereof or increase in intestine crypt or villus length is determined after a test animal or vertebrate has been treated or exposed to a long acting GLP-2 analog.

In one embodiment, compounds of the present invention increase height of the crypt plus villus axis or increase crypt cell proliferation or decrease small bowel epithelium apoptosis in a patient.

In one embodiment, compounds of the present invention increase crypt/villus height. In another embodiment, compounds of the present invention increase crypt/villus height in the jejunum, including the proximal jejunum, distal jejunum, and distal ileum.

In one embodiment, compounds of the present invention increase crypt cell proliferation or decrease small bowel epithelium apoptosis.

The present invention includes the following peptides further described in the experimental section below.

GLP-2-G1y2:
(SEQ ID NO: 1)
NH2-HGDGSFSDEMNTILDNLAARDFINWLIQTKITD-COOH.
GLP-2 Variant #2:
(SEQ ID NO: 2)
NH2-HGEGSFSDE(Nle)(D-F)TILDNLAARDFINWLIQTKITD-
NH2.
GLP-2 Variant #3:
(SEQ ID NO: 3)
NH2-HGEGSFSDE(Nle)(D-H)TILDNLAARDFINWLIQTKITD-
NH2.
GLP-2 Variant #4:
(SEQ ID NO: 4)
NH2-HGEGSFSDE(Nle)NTILDLLAARDFINWLIQTKITD-NH2.
GLP-2 Variant #5:
(SEQ ID NO: 5)
NH2-HGEGSFSDE(Nle)NTILDYLAARDFINWLIQTKITD-NH2.
GLP-2 Variant #6:
(SEQ ID NO: 6)
NH2-HGEGSFSDE(Nle)NTILDLLAARDFINWLIQTKITD-COOH.
GLP-2 Variant #7:
(SEQ ID NO: 7)
NH2-HGEGSFSDE(Nle)NTILDYLAARDFINWLIQTKITD-COOH.
Human GLP-2 is known to have the following
sequence:
(SEQ ID NO: 8)
NH2-HADGSFSDEMNTILDNLAARDFINWLIQTKITD-COOH.

In one embodiment, the name “teduglutide” is used to refer to a glucagon-like peptide-2 (GLP-2) analogue made up of 33 amino acids which differs from GLP-2 by one amino acid (alanine at AA position 2 is substituted by glycine). In one embodiment, this substitution is results in longer action compared with endogenous GLP-2 due to its increase resistance to proteolysis from dipeptidyl peptidase-4.

A wide variety of useful active GLP-2 analogs and derivatives have been described in the literature, as revealed in: U.S. Pat. No. 5,789,379 issued Jun. 20, 2000 and related WO97/39031 published Oct. 23, 1997 which teach site-specific GLP-2 analogs; WO02/066511 published Aug. 27, 2003 which teaches albumin-derivatized forms of GLP-2 and analogs; WO99/43361 published Oct. 14, 1999, WO04/035624 published Apr. 29, 2004 and WO04/085471 published Oct. 7, 2004 which describe lipophilic-derivatized forms of GLP-2 and analogs; and U.S. Pat. No. 9,060,992 issued Jun. 23, 2015, which describe GLP-2 analogs. GLP-2 analogs are also described in U.S. Pat. Nos. 8,642,727, 9,453,064, 8,580,918, WO 2013/183052, WO 2016/193969, and WO 2012/167251.

In some embodiments, a GLP-2 analogue is represented by the following formula:

R1-His-X2-X3-Gly-X5-Phe-X7-X8-X9-X10-X11-X12-X13-X14-X15-X16-X17-Ala-X19-X20-X21-Phe-Ile-X24-Trp-Leu-X27-X28-X29-X30-X31-X32-X33-R2 (SEQ ID NO: 9), wherein:

R1 is hydrogen, C1-4 alkyl (e.g. methyl), acetyl, formyl, benzoyl, trifluoroacetyl, OH, COOH, NH₂, CONH₂, or CONHNH₂;

### X2 is Gly, Ala or Aib;

### X3 is Glu, Gln or Asp;

### X5 is Ser or Thr;

### X7 is Ser or Thr;

### X8 is Asp, Glu or Ser;

### X9 is Glu or Asp;

### X10 is Met, Val, Leu or Tyr;

### X11 is Asn, Ser or Ala;

### X12 is Thr, Ser or Lys;

### X13 is Ile, Leu, Val, Tyr, Phe or Gln;

### X14 is Leu or Met;

### X15 is Asp or Glu;

### X16 is Asn, Gln, Gly, Ser, Ala, Glu or Lys;

### X17 is Gln, Lys, Arg, His or Glu;

### X19 is Ala or Val;

### X20 is Arg, Lys or His;

### X21 is Asp, Glu or Leu;

### X24 is Asn, Ala, Glu or Lys;

### X27 is Ile, Leu, Val, Glu or Lys;

X28 is Gln, Asn, Lys, Ser, Y1 or absent;
X29 is Thr, Y1 or absent;
X30 is Lys, Y1 or absent;
X31 is Ile, Pro or absent;
X32 is Thr, Y1 or absent;
X33 is Asp, Asn, Y1 or absent;

### Y1 is Gly-Gly-Pro-Ser-Ser-Gly-Ala-Pro-Pro-Pro-Ser, or Lys-Asn-Gly-Gly-Pro-Ser-Ser-Gly-Ala-Pro-Pro-Pro-Ser; and

R2 is OH, COOH, NH₂, CONH₂, or CONHNH₂.

In some embodiments, in SEQ ID NO: 9, X31 may also be Y1; X28 may also be Gly; or X29 may also be Ala. Additionally, Y1 may be present between X33 and R2. Thus, a position X34 may be envisaged, where X34 is Y1 or is absent.

In some embodiments, a GLP-2 analogue is represented by the following formula:

R1-Z1-His-X2-X3-Gly-X5-X6-X7-X8-X9-X10-X11-X12-X13-X14-X15-X16-X17-X18-X19-X20-X21-Phe-Ile-X24-Trp-Leu-Ile-X28-Thr-Lys-X31-X32-X33-Z2-R2 (SEQ ID NO: 10), wherein:

R1 is hydrogen, C1-4 alkyl (e.g. methyl), acetyl, formyl, benzoyl or trifluoroacetyl;

### X2 is Gly, Ala or Sar;

### X3 is Glu or Asp;

### X5 is Ser or Thr;

X6 is Phe or Pro or a conservative substitution;

### X7 is Ser or Thr;

X8 is Asp or Ser or a conservative substitution;
X9 is Glu or Asp or a conservative substitution;
X10 is Met, Leu, Nle or an oxidatively stable Met-replacement amino acid;

### X11 is Y1;

X12 is Thr or Lys or a conservative substitution;
X13 is Be, Glu or Gln or a conservative substitution;
X14 is Leu, Met or Nle or a conservative substitution;
X15 is Asp or Glu or a conservative substitution;

### X16 is Y2;

X17 is Leu or Glu or a conservative substitution;
X18 is Ala or Aib or a non-conservative substitution;
X19 is Ala or Thr or a conservative substitution;

### X20 is Y3

X21 is Asp or Ile or a conservative substitution;

### X24 is Y4;

### X28 is Y5;

X31 is Pro, Ile or deleted;
X32 is Thr or deleted;
X33 is Asp, Asn or deleted;
Y1, Y2, Y4, and Y5 can individually be selected from the group consisting of Asn, Asp, Glu, Gln, Lys, His, Arg, Ala, Ser, Thr, Pro, Gly, Leu, Ile, Val, Met or Phe; and Y3 can be selected from the group consisting of Asn, Asp, Glu, Gln, His, Arg, Ala, Ser, Thr, Pro, Gly, Leu, Ile, Val, Met or Phe; R2 is NH2 or OH; and
Z1 and Z2 are independently absent or a peptide sequence of 1-10 amino acid units selected from the group consisting of Ala, Leu, Ser, Thr, Tyr, Asn, Gln, Asp, Glu, Lys, Arg, His, Met and Orn.

In one embodiment, the GLP-2 analog is selected from the group consisting of:

(SEQ ID NO: 11)
R1-His-Gly-Asp-Gly-Ser-Phe-Ser-Asp-Glu-Nle-D-Thi-
Thr-Ile-Leu-Asp-Phe-Leu-Ala-Ala-Arg-Asp-Phe-Ile-
Asn-Trp-Leu-Ile-Gln-Thr-Lys-R2;
(SEQ ID NO: 12)
R1-His-Gly-Asp-Gly-Ser-Phe-Ser-Asp-Glu-Nle-D-Phe-
Thr-Ile-Leu-Asp-Phe-Leu-Ala-Ala-Arg-Asp-Phe-Ile-
Asn-Trp-Leu-Ile-Gln-Thr-Lys-R2;
(SEQ ID NO: 13)
R1-His-Gly-Asp-Gly-Ser-Phe-Ser-Asp-Glu-Nle-D-Phe-
Thr-Ile-Leu-Asp-Leu-Leu-Ala-Ala-Arg-Asp-Phe-Ile-
Asn-Trp-Leu-Ile-Gln-Thr-Lys-Ile-Thr-Asp-R2;
(SEQ ID NO: 14)
R1-His-Gly-Asp-Gly-Ser-Phe-Ser-Asp-Glu-Nle-D-Thi-
Thr-Ile-Leu-Asp-Leu-Leu-Ala-Thr-Arg-Asp-Phe-Ile-
Asn-Trp-Leu-Ile-Gln-Thr-Lys-Ile-Thr-Asp-R2;
(SEQ ID NO: 15)
R1-His-Gly-Asp-Gly-Ser-Phe-Ser-Asp-Glu-Nle-D-Phe-
Thr-Ile-Leu-Asp-Phe-Leu-Ala-Ala-Arg-Asp-Phe-Ile-
Asn-Trp-Leu-Ile-Gln-Thr-Lys-Ile-Thr-Asp-R2; or
(SEQ ID NO: 16)
R1-His-Gly-Asp-Gly-Ser-Phe-Ser-Asp-Glu-Nle-D-Thi-
Thr-Ile-Leu-Asp-Phe-Leu-Ala-Ala-Arg-Asp-Phe-Ile-
Asn-Trp-Leu-Ile-Gln-Thr-Lys-Ile-Thr-Asp-R2,

wherein

R1 is OH, COOH, NH₂, CONH₂, or CONHNH₂, and

R2 is OH, COOH, NH₂, CONH₂, NH-isobutyl, or CONHNH₂.

In one embodiment provided herein is a method for extending the biological half-life of GLP-2 by substituting, deleting, inverting or adding one or more amino acids compared with native GLP-2. In another embodiment, provided herein is a method for extending the biological half-life of GLP-2 by incorporating at least one amino acid substitution at the positions X2, X3, X10, X11, and X16, wherein the GLP-2 analog has following amino acid sequence:

(SEQ ID NO: 17)
R1-His1-X2-X3-Gly4-Ser5-Phe6-Ser7-Asp8-G1u9-X10-
X11-Thr12-Ile13-Leu14-Asp15-X16-Leu17-Ala18-
Ala19-Arg20-Asp21-Phe22-Ile23-Asn24-Trp25-Leu26-
Ile27-Gln28-Thr29-Lys30-Ile31-Thr32-Asp33-R2

In another embodiment, provided herewith is a method for extending the biological half-life of GLP-2 by incorporating at least one amino acid substitution and the resulting GLP-2 analog has the amino acid sequence of SEQ ID NO: 17 and

R1 is OH, COOH, NH₂, CONH₂, or CONHNH₂;

X2 is Ala or Gly;

X3 is Asp or Glu;

X10 is Met or Nle;

X11 is Asn, D-Phe, or D-His;

X16 is Asn, Leu, or Tyr;

R2 is OH, COOH, NH₂, CONH₂, or CONHNH₂.

In one embodiment provided herein is a method for reducing the dosing frequency of GLP-2 by substituting, deleting, inverting or adding one or more amino acids compared with native GLP-2. In another embodiment, provided herein is a method for reducing the dosing frequency of GLP-2 by incorporating at least one amino acid substitution at the positions X2, X3, X10, X11, and X16, wherein the GLP-2 analog has following amino acid sequence of SEQ ID NO: 17

In another embodiment, provided herewith is a method for extending the reducing the dosing frequency of GLP-2 by incorporating at least one amino acid substitution and the resulting GLP-2 analog has the amino acid sequence of SEQ ID NO: 17 and

R1 is OH, COOH, NH₂, CONH₂, or CONHNH₂;

X2 is Ala or Gly;

X3 is Asp or Glu;

X10 is Met or Nle;

X11 is Asn, D-Phe, or D-His;

X16 is Asn, Leu, or Tyr; or

R2 is OH, COOH, NH₂, CONH₂, or CONHNH₂.

In another embodiment, provided herein is a method for extending the biological half-life of a GLP-2 analog by attaching 9-fluorenylmethoxycarbonyl (Fmoc), a maleimide moiety of Fmoc (MAL-Fmoc), 2-sulfo-9-fluorenylmethoxycarbonyl (FMS), a maleimide moiety of FMS (MAL-FMS), 2-methoxy-9-fluorenylmethoxycarbonyl (MeOFmoc), or NRFmoc at one or more amino acid positions of the GLP-2 analog. In another embodiment, provided herein is a method for extending the biological half-life of a GLP-2 analog by attaching 9-fluorenylmethoxycarbonyl (Fmoc), MAL-Fmoc 2-sulfo-9-fluorenylmethoxycarbonyl (FMS), MAL-FMS, 2-methoxy-9-fluorenylmethoxycarbonyl (MeOFmoc), or NRFmoc to the amino terminus or to the lysine residue on position number thirty (Lys30), or to the His (1) imidazole side chain, or any combination of them, of the GLP-2 analog.

In another embodiment, provided herein is a method for reducing the dosing frequency of a GLP-2 analog by attaching 9-fluorenylmethoxycarbonyl (Fmoc), a maleimide moiety of Fmoc (MAL-Fmoc), 2-sulfo-9-fluorenylmethoxycarbonyl (FMS), a maleimide moiety of FMS (MAL-FMS), MeOFmoc, or NRFmoc at one or more amino acid positions of the GLP-2 analog. In another embodiment, provided herein is a method for reducing the dosing frequency of a GLP-2 analog by attaching 9-fluorenylmethoxycarbonyl (Fmoc), MAL-FMoc, 2-sulfo-9-fluorenylmethoxycarbonyl (FMS), MAL-FMS, MeOFmoc, or NRFmoc to the amino terminus, to the lysine residue on position number thirty (Lys30), or to the His (1) imidazole side chain, or any combination of them, of the GLP-2 analog.

In another embodiment, provided herein is a method for improving the biological efficacy of GLP-2 analog by attaching 9-fluorenylmethoxycarbonyl (Fmoc), MAL-Fmoc, 2-sulfo-9-fluorenylmethoxycarbonyl (FMS), MAL-FMS, MeOFmoc, or NRFmoc at one or more amino acid positions of the GLP-2 analog. In another embodiment, provided herein is a method for extending the biological half-life of a GLP-2 analog by attaching 9-fluorenylmethoxycarbonyl (Fmoc), MAL-Fmoc, 2-sulfo-9-fluorenylmethoxycarbonyl (FMS), MAL-FMS, MeOFmoc, or NRFmoc to the amino terminus, to the lysine residue on position number thirty (Lys30), or to the His (1) imidazole side chain, or any combination, of the GLP-2 analog.

In another embodiment, provided herein is a method for improving the biological efficacy and/or extending the biological half-life of a GLP-2 analog by attaching 9-fluorenylmethoxycarbonyl (Fmoc), MAL-Fmoc, 2-sulfo-9-fluorenylmethoxycarbonyl (FMS), MAL-FMS, MeOFmoc, or NRFmoc to the amino terminus, to the lysine residue on position number thirty (Lys30), the His residue on position number one (His1), or any combination, of the GLP-2 analog.

In another embodiment provided herein is a method for reducing the dosing frequency, extending the biological half-life, or improving the biological efficacy of the GLP-2 analogues described herein by attaching an Fmoc-Osu linker to the GLP-2 analog via any of the free amines potentially located at the N-terminal and/or the Lys 30. Fmoc-Osu structure is described below in Formula I. In another embodiment the Fmoc-Osu linker is sulfonated.

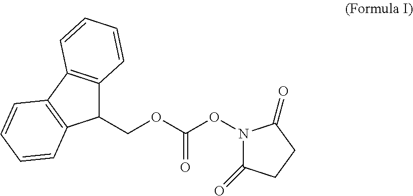 [SMILES: O=C(OCC1c2ccccc2-c2ccccc21)ON1C(=O)CCC1=O]

In one embodiment, Fmoc-Osu is a mono-functional linker. In another embodiment, the Fmoc-Osu linker is covalently bound to a GPL-2 analog via a carbamate bond. In another embodiment, other potential interactions (e.g, hydrophobic interactions) between the linker moieties and other bio-molecules are non-covalent-based.

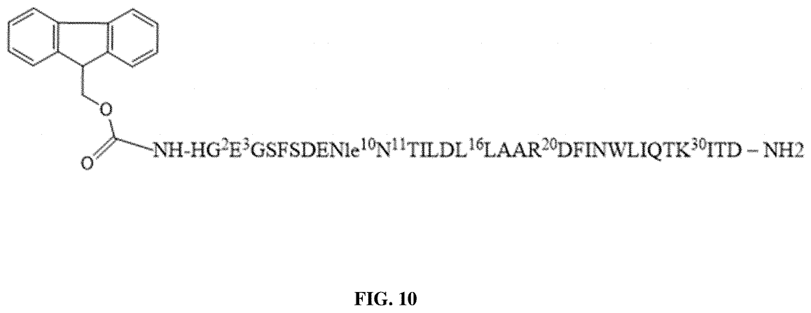

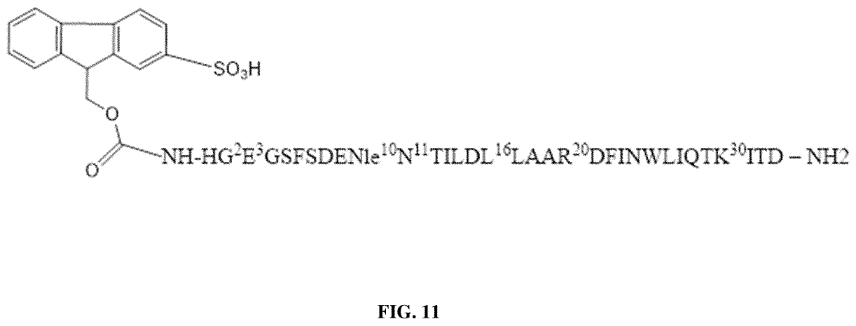

In one embodiment, the structure of the Fmoc-Osu linker following coupling to the GLP-2 analog is described in FIG. 10. In one embodiment, the structure of the FMS-Osu linker following coupling to the GLP-2 analog is described in FIG. 11.

In one embodiment, MAL-Fmoc-OR of this invention is presented by the following structure.

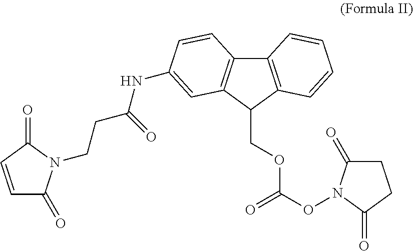 [SMILES: O=C(CCN1C(=O)C=CC1=O)Nc1ccc2c(c1)C(COC(=O)ON1C(=O)CCC1=O)c1ccccc1-2]

In one embodiment, MAL-FMS-OR of this invention is presented by the following structure.

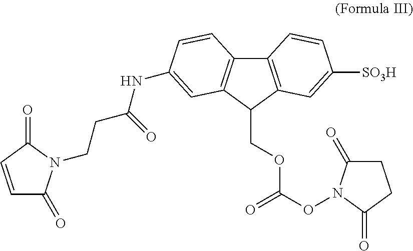 [SMILES: O=C(CCN1C(=O)C=CC1=O)Nc1ccc2c(c1)C(COC(=O)ON1C(=O)CCC1=O)c1cc(S(=O)(=O)O)ccc1-2]

In one embodiment, MeOFmoc of this invention is presented by the following structure.

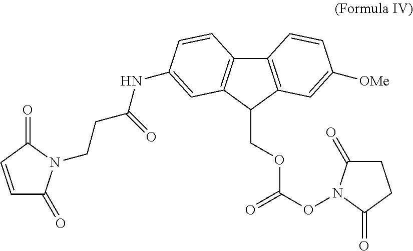 [SMILES: COc1ccc2c(c1)C(COC(=O)ON1C(=O)CCC1=O)c1cc(NC(=O)CCN3C(=O)C=CC3=O)ccc1-2]

In one embodiment, NRFmoc of this invention is presented by the following structure.

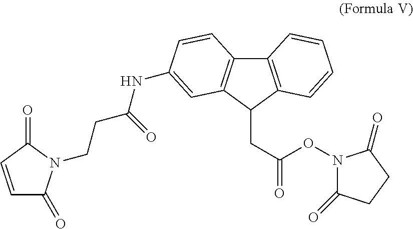 [SMILES: O=C(CCN1C(=O)C=CC1=O)Nc1ccc2c(c1)C(CC(=O)ON1C(=O)CCC1=O)c1ccccc1-2]

In one embodiment, the maleimide moiety MAL-FMS-NHS of this invention is presented by the following structure.

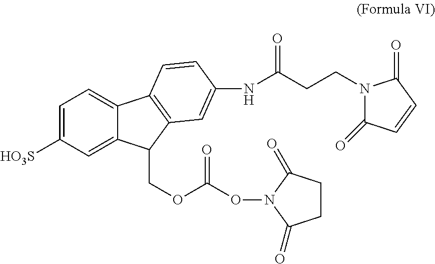 [SMILES: O=C(CCN1C(=O)C=CC1=O)Nc1ccc2c(c1)C(COC(=O)ON1C(=O)CCC1=O)c1cc(S(=O)(=O)O)ccc1-2]

In one embodiment, the MAL-FMS-NHS is prepared by mixing MAL-Fmoc-NHS with trifluoroacetic acid and chlorosulfonic acid, wherein said MAL-Fmoc-NHS is dissolved in neat trifluoroacetic acid, and an excess of said chlorosulfonic acid dissolved in neat trifluoroacetic acid is added to the reaction mixture.

In one embodiment, the maleimide moiety MAL-Fmoc-NHS of this invention is presented by the following structure.

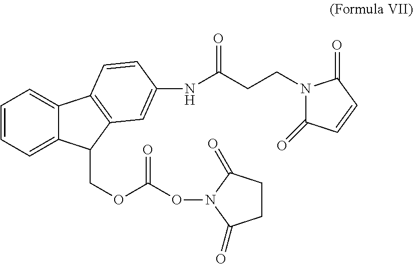 [SMILES: O=C(CCN1C(=O)C=CC1=O)Nc1ccc2c(c1)C(COC(=O)ON1C(=O)CCC1=O)c1ccccc1-2]

In one aspect, the invention provides a composition comprising or consisting of GLP-2 analogs linked to one or more polyethylene glycol polymers (PEG) via a reversible linker, such as 9-fluorenylmethoxycarbonyl (Fmoc), a maleimide moiety of Fmoc (MAL-Fmoc), 2-sulfo-9-fluorenylmethoxycarbonyl (FMS), a maleimide moiety of FMS (MAL-FMS), or MeOFmoc. In another embodiment, the invention provides a composition comprising or consisting of a GLP-2 analog, a polyethylene glycol polymer (PEG polymer) and 9-fluorenylmethoxycarbonyl (Fmoc), MAL-Fmoc, 2-sulfo-9-fluorenylmethoxycarbonyl (FMS), MAL-FMS, or MeOFmoc.

In one embodiment, the invention provides a composition comprising or consisting of GLP-2 analogs linked to one or more polyethylene glycol polymers (PEG) via an irreversible linker, such as 2,5-dioxopyrrolidin-1-yl-3-(2-(3-(2,5-dioxo-2,5-dihydro-1H-pyrrol-1-yl)propanamido)-9H-fluoren-9-yl)propanoate (NRFmoc).

In one embodiment, where the linker is reversible it is bonded to a peptide through a carbamate bond (i.e. —O—C(═O)—N(H)—).

In one embodiment, where the linker is irreversible it is bonded to a peptide through an amid bond (i.e. —C(═O)—N(H)—).

In another aspect, provided herein is a method for extending the serum half-life of peptides. This method is based on the reversible attachment of a polyethylene glycol (PEG) chain to the peptide through a chemical linker (called FMS, MAL-FMS, Fmoc, MAL-Fmoc, or MeOFmoc) resulting in the slow release of the native peptide into the bloodstream. The released peptide can then also cross the blood brain barrier to enter the central nervous system (CNS) or any other target organ. In one embodiment, the unique chemical structure of the FMS, MAL-FMS, Fmoc, MAL-Fmoc, or MeOFmoc linker leads to a specific rate of peptide release.

Hence, in another embodiment, provided herein is a method for extending the biological half-life of a GLP-2 analog. In another embodiment, provided herein is a method for extending the circulating time in a biological fluid of a GLP-2 analog, wherein said circulating time is extended by the slow release of the intact a GLP-2 analog. In another embodiment, extending said biological half-life or said circulating time of said a GLP-2 analog allows said a GLP-2 analog to reduce gastric motility and gastric acid secretion (See Drucker, Daniel J., and Bernardo Yusta. “Physiology and pharmacology of the enteroendocrine hormone glucagon-like peptide-2.” Annual review of physiology 76 (2014): 561-583)). It will be well appreciated by the skilled artisan that the biological fluid may be blood, sera, cerebrospinal fluid (CSF), and the like.

In one embodiment, the long acting GLP-2 analogs of the invention are mediated by a G protein-coupled receptor. In one embodiment, the long acting GLP-2 analogs of the invention are mediated by the GLP-2 receptor (GLP-2R).

In one embodiment, upon administration of the PEGylated GLP-2 analog composition of the present invention into a subject, the GLP-2 analog is released into a biological fluid in the subject as a result of chemical hydrolysis of said FMS, MAL-FMS, Fmoc, or MAL-Fmoc linker from said composition. In another embodiment, the released GLP-2 analog is intact and regains complete GLP-2 receptor binding activity. In another embodiment, chemically hydrolyzing said FMS, MAL-FMS, Fmoc, or MAL-Fmoc extends the circulating time of said GLP-2 analog in said biological fluid. In another embodiment, extending the circulating time of said GLP-2 analog allows said GLP-2 analog to cross the blood brain barrier and target the CNS. In another embodiment, extending the circulating time of said GLP-2 analog allows said GLP-2 analog to cross the blood brain barrier and target the hypothalamus. In another embodiment, extending the circulating time of said GLP-2 analog allows said GLP-2 analog to cross the blood brain barrier and target the arcuate nucleus.

In another embodiment, the present invention provides a composition comprising a GLP-2 analog peptide, and a polyethylene glycol (PEG) polymer conjugated to the amino terminus of the GLP-2 analog peptide via a 9-fluorenylmethoxycarbonyl (Fmoc), MAL-Fmoc, 2-sulfo-9-fluorenylmethoxycarbonyl (FMS), MAL-FMS, or MeOFmoc linker. In another embodiment, the invention relates to a composition consisting of a GLP-2 analog, a polyethylene glycol polymer (PEG polymer), and a 9-fluorenylmethoxycarbonyl (Fmoc), MAL-Fmoc, 2-sulfo-9-fluorenylmethoxycarbonyl (FMS), MAL-FMS, or MeOFmoc linker, wherein said PEG polymer is attached to a lysine residue on position number thirty (Lys30) of said GLP-2's amino acid sequence via Fmoc, MAL-Fmoc, FMS, MAL-FMS, or MeOFmoc. In another embodiment, the invention relates to a composition consisting of a GLP-2 analog, a polyethylene glycol polymer (PEG polymer), and a 9-fluorenylmethoxycarbonyl (Fmoc), MAL-Fmoc, 2-sulfo-9-fluorenylmethoxycarbonyl (FMS), MAL-FMS, or MeOFmoc linker, wherein said PEG polymer is attached to the His (1) imidazole side chain of said GLP-2's amino acid sequence via Fmoc, MAL-Fmoc, FMS, MAL-FMS, or MeOFmoc.

In one embodiment, the present invention provides a heterologous composition comprising a GLP-2 analog attached to a polyethylene glycol polymer (PEG polymer) via a 9-fluorenylmethoxycarbonyl (Fmoc), MAL-Fmoc, 2-sulfo-9-fluorenylmethoxycarbonyl (FMS), MAL-FMS, or MeOFmoc linker at the lysine residue on position number thirty (Lys30) of said GLP-2's amino acid sequence, and a GLP-2 analog peptide attached to a polyethylene glycol (PEG) polymer via a Fmoc, MAL-Fmoc, FMS, MAL-FMS, or MeOFmoc linker at the amino terminus of the GLP-2 analog peptide. In another embodiment, the present invention provides a heterologous composition comprising: (1) a GLP-2 analog attached to a polyethylene glycol polymer (PEG polymer) via a 9-fluorenylmethoxycarbonyl (Fmoc), MAL-Fmoc, 2-sulfo-9-fluorenylmethoxycarbonyl (FMS), MAL-FMS, or MeOFmoc linker at the lysine residue on position number thirty (Lys30) of said GLP-2's amino acid sequence; (2) a GLP-2 analog peptide attached to a polyethylene glycol (PEG) polymer via a Fmoc, MAL-Fmoc, FMS, MAL-FMS, or MeOFmoc linker at the amino terminus of the GLP-2 analog peptide; and/or (3) a GLP-2 analog peptide attached to a polyethylene glycol (PEG) polymer via a Fmoc, MAL-Fmoc, FMS, MAL-FMS, or MeOFmoc linker at the His (1) imidazole side chain. of the GLP-2 analog peptide.

In another embodiment, a long-acting GLP-2 analog is a pegylated GLP-2 analog. In another embodiment, a long-acting GLP-2 analog is a reversed pegylated GLP-2 analog. In another embodiment, the phrases “long-acting GLP-2 analog,” “reversed pegylated GLP-2 analog,” “reversable PEGylated GLP-2 analog,” or “a composition comprising or consisting of GLP-2 analog, polyethylene glycol polymer (PEG polymer) and 9-fluorenylmethoxycarbonyl (Fmoc), MAL-Fmoc, 2-sulfo-9-fluorenylmethoxycarbonyl (FMS), MAL-FMS, or MeOFmoc” are used interchangeably. In another embodiment, a long-acting GLP-2 analog is GLP-2 analog linked to PEG via Fmoc, MAL-Fmoc, FMS, MAL-FMS, or MeOFmoc. In another embodiment, the long-acting GLP-2 analog is linked to Fmoc, MAL-Fmoc, FMS, MAL-FMS, or MeOFmoc via its amino (N′) terminus. In another embodiment, the long-acting GLP-2 analog is linked to Fmoc, MAL-Fmoc, FMS, MAL-FMS, or MeOFmoc via its His (1) imidazole side chain.

In one aspect, the invention provides a composition comprising or consisting of a GLP-2 analog reversibly PEGylated via a MAL-Fmoc or MAL-FMS linker. In a further aspect, the GLP-2 analog reversibly PEGylated via a MAL-Fmoc or MAL-FMS linker can be further conjugated to another molecule in addition to the PEG. In another embodiment, the additional conjugated molecule is a thiol-containing molecule. In another embodiment, the additional conjugated molecule is a SH active group or an amine, hydrazine, or hydrazide. In another embodiment, the additional conjugated molecule is Cys or cysteamine.

In another embodiment, the GLP-2 analogs provided herein have 9-fluorenylmethoxycarbonyl (Fmoc), MAL-Fmoc, 2-sulfo-9-fluorenylmethoxycarbonyl (FMS), MAL-FMS, or MeOFmoc attached to one or more amino acid positions of the GLP-2 analog. In another embodiment, the GLP-2 analogs provided herein have 9-fluorenylmethoxycarbonyl (Fmoc), MAL-Fmoc, 2-sulfo-9-fluorenylmethoxycarbonyl (FMS), MAL-FMS, or MeOFmoc attached to the amino terminus or to the lysine residue on position number thirty (Lys30) of the GLP-2 analog. In another embodiment, the present invention provides a heterologous composition comprising a GLP-2 analog with 9-fluorenylmethoxycarbonyl (Fmoc), MAL-Fmoc, 2-sulfo-9-fluorenylmethoxycarbonyl (FMS), MAL-FMS, or MeOFmoc attached to the amino terminus of the GLP-2 analog and a GLP-2 analog with 9-fluorenylmethoxycarbonyl (Fmoc), MAL-Fmoc, 2-sulfo-9-fluorenylmethoxycarbonyl (FMS), MAL-FMS, or MeOFmoc attached to the lysine residue on position number thirty (Lys30) of the GLP-2 analog.

In another embodiment, a reverse pegylated GLP-2 analog is a composition wherein GLP-2 analog is linked to PEG via a reversible linker. In another embodiment, a reverse pegylated GLP-2 analog releases a free GLP-2 analog upon exposure to a natural to basic environment. In another embodiment, a reverse pegylated GLP-2 analog releases a free GLP-2 analog upon exposure to blood or plasma. In another embodiment, a long-acting GLP-2 analog comprises PEG and GLP-2 analog that are not linked directly to each other, as in standard pegylation procedures, but rather both residues are linked to different positions of Fmoc, MAL-Fmoc, FMS, or MAL-FMS which are highly sensitive to pH conditions and are removable under regular physiological conditions. In another embodiment, regular physiological conditions include a physiologic environment such as the blood or plasma.

In another embodiment, the structures and the processes of making Fmoc, MAL-Fmoc, FMS, and MAL-FMS are described in U.S. Pat. No. 7,585,837. The disclosure of U.S. Pat. No. 7,585,837 is hereby incorporated by reference in its entirety.

In another aspect, provided herein is a method of reducing the dosing frequency of a GLP-2 analog, consisting of the step of conjugating a polyethylene glycol polymer (PEG polymer) to the Lysine residue on position number 30, to the N terminus, or to the His (1) side chain of the GLP-2 analog sequence via 9-fluorenylmethoxycarbonyl (Fmoc), MAL-Fmoc, 2-sulfo-9-fluorenylmethoxycarbonyl (FMS), MAL-FMS, or MeOFmoc.

In one embodiment, the maleimide moiety linkers of the present invention are hydrogenated.

In one embodiment, the maleimide moiety linkers have one or more maleimide groups replaced with a succinimide group.

In one embodiment, the linkers containing a succinimide group have the following structure:

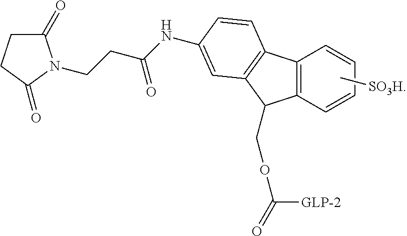

In another aspect, provided herein is a method of reducing the dosing frequency of a GLP-2 analog, due to the improved efficacy of a long acting GLP-2 analog as described herein. In another aspect, provided herein is a method of reducing the dosing frequency and/or increasing the efficacy of the GLP-2 or GLP-2 analog, consisting of the step of conjugating at least one linker said Fmoc, MAL-Fmoc, FMS, MAL-FMS, MeOFmoc, or NRFmoc or combination thereof to the GLP-2 peptide or GLP-2 analog at the N terminal, Lys (30) side chain, or His (1) side chain, or any combination thereof, and further reducing the maleimide functional group using, but not limit to thiol-containing molecules (e.g, cysteine and cysteamine), amine-containing molecules, and hydrogenation. In another embodiment, reacting the thiol-containing molecule with the GLP-2 analog results in the reduction of the MAL-linker-GLP-2 such as maleimide hydrogenation, and/or the coupling of the thiol-containing molecule to the linker-GLP-2.

In one embodiment aspect, provided herein is a method of extending the half-life of the GLP-2 analog, consisting of the step of conjugating at least one linker such as Fmoc, MAL-Fmoc, FMS, MAL-FMS, MeOFmoc, or NRFmoc, or combination thereof to the GLP-2 peptide or GLP-2 peptide analog at the N terminal, Lys (30) side chain, or His (1) side chin, or any combination thereof, and further reducing the maleimide functional group using, but not limit to thiol-containing molecules (e.g, cysteine (“Cys”) and cysteamine), amine-containing molecules, and hydrogenation.

In another aspect, provided herein is a method of improving the area under the curve (AUC) of a GLP-2 analog, consisting of the step of conjugating a polyethylene glycol polymer (PEG polymer) to the Lysine residue on position number 30, the N terminus, or the His (1) imidazole side chain of the GLP-2 analog sequence via a 9-fluorenylmethoxycarbonyl (Fmoc), MAL-Fmoc, 2-sulfo-9-fluorenylmethoxycarbonyl (FMS), MAL-FMS, or MeOFmoc linker.

In one aspect, provided herein is a method of improving the area under the curve (AUC) of a GLP-2 analog, consisting of the step of conjugating a 9-fluorenylmethoxycarbonyl (Fmoc), MAL-Fmoc, 2-sulfo-9-fluorenylmethoxycarbonyl (FMS), MAL-FMS, MeOFmoc, or NRFmoc linker to the Lysine residue on position number 30, the N terminus, or the His (1) imidazole side chain of the GLP-2 analog.

In another aspect, provided herein is a method of improving the area under the curve (AUC) of a GLP-2 analog, consisting of the step of irreversibly conjugating a polyethylene glycol polymer (PEG polymer) to the Lysine residue on position number 30, the N terminus, or the His (1) imidazole side chain of the GLP-2 analog sequence via a NRFmoc linker.

In another embodiment, PEG is linear. In another embodiment, PEG is branched. In another embodiment, PEG has a molecular weight in the range of 1 to 200 Da. In another embodiment, PEG has a molecular weight in the range of 200 to 200,000 Da. In another embodiment, PEG has a molecular weight in the range of 5,000 to 80,000 Da. In another embodiment, PEG has a molecular weight in the range of 5,000 to 40,000 Da. In another embodiment, PEG has a molecular weight in the range of 20,000 Da to 40,000 Da. In one embodiment, PEG₂₀ refers to a PEG with an average molecular weight of 20,000 Da. In one embodiment, PEG₅ refers to a PEG with an average molecular weight of 5,000 Da. In one embodiment, PEG₃₀ refers to a PEG with an average molecular weight of 30,000 Da. PEG40 refers to a PEG with an average molecular weight of 40,000 Da.

In one embodiment, PEG has a molecular weight of about 2,000 Da. In another embodiment, PEG has a molecular weight of about 1,000 Da. In another embodiment, PEG has a molecular weight of about 5000 Da. In another embodiment, PEG has a molecular weight of about 100 Da. In another embodiment, PEG has a molecular weight in the range of 1 to 500 Da. In another embodiment, PEG has a molecular weight in the range of 500 to 1,000 Da. In another embodiment, PEG has a molecular weight in the range of 1,000 to 2,000 Da. In another embodiment, PEG has a molecular weight in the range of 2,000 to 5,000 Da.

In another embodiment, the polyethylene glycol is a branched PEG represented as (PEG)m-R—SH in which R represents a central core moiety and m represents the number of branching arms. In one embodiment, the PEG is represented as (PEG)m-R—SH with only one available connection to the polypeptide. The number of branching arms (m) can range from two to a hundred or more. In another embodiment, the hydroxyl groups are subject to chemical modification. In another embodiment the branched PEG has an average molecular weight of 20 KD or 40 KD and is represented as (PEG)2-R—SH.

In another embodiment, the branched PEG is represented as (PEG)2-R—SH and has the following chemical structure:

In another embodiment, the PEG is a multi-arm PEG represented as (PEG)4-R—SH. In one embodiment, the PEG is a multi-arm PEG represented as (PEG)4-R—SH in which each PEG arm has a molecular weight of 20 KD or 40 KD.

In another embodiment, the PEG is a multi-arm PEG represented by the following chemical structure:

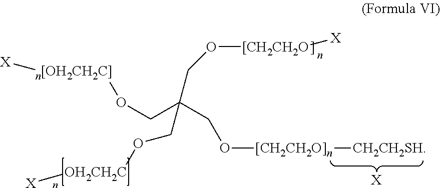

In another embodiment, the PEG is a multi-arm PEG represented by formula 1 above and each PEG arm has an average molecular weight of 20 KD or 40 KD.

In another embodiment, branched PEGs are represented as R(PEG-OH)ₘ in which R represents a central core moiety such as pentaerythritol or glycerol, and m represents the number of branching arms. The number of branching arms (m) can range from two to a hundred or more. In another embodiment, the hydroxyl groups are subject to chemical modification. In another embodiment, branched PEG molecules are described in U.S. Pat. Nos. 6,113,906, 5,919,455, 5,643,575, and 5,681,567, which are hereby incorporated by reference in their entirety.

In another embodiment, a long-acting GLP-2 analog is prepared using PEGylating agents, meaning any PEG derivative which is capable of reacting with a functional group such as, but not limited to, NH2, OH, SH, COOH, CHO, —N═C═O, —N═C═S, —SO2C1, —SO2CH═CH2, —PO2C1, —(CH2)xHal, present at the fluorene ring of the Fmoc, MAL-Fmoc, FMS, or MAL-FMS moiety. In another embodiment, the PEGylating agent is usually used in its mono-methoxylated form where only one hydroxyl group at one terminus of the PEG molecule is available for conjugation. In another embodiment, a bifunctional form of PEG where both termini are available for conjugation may be used if, for example, it is desired to obtain a conjugate with two peptide or protein residues covalently attached to a single PEG moiety.

In another embodiment the invention relates to therapeutic and related uses of GLP-2 analogs, GLP-2 analogs linked solely to Fmoc, MAL-Fmoc, FMS, MAL-FMS or MeOFmoc, or NRFmoc for reversibly or non-reversibly PEGylated GLP-2 analogs, particularly to promote the growth of small and/or large intestine tissue; elevate blood levels of GLP-2 derivative; restore or maintain gastrointestinal function; promote the healing and regrowth of injured or ulcerated/inflamed intestinal mucosa; reduce the risk of enteric disease; enhance the nutritional status; treat or prevent nutritional or gastrointestinal disorders, complications or diseases; reduce weight loss; reduce interleukin-1 expression; increase colon length, both mucosal area and integrity in the colon, and crypt depth; promote villous growth in subjects suffering from a disease such as celiac disease, post-infectious villous atrophy and short gut syndromes; promote proliferation of the small and large intestine in a healthy subject or a subject with a disease. The effect on growth elicited by the long acting GLP-2 analogs manifests as an increase in small bowel weight, relative to a mock-treated control. In particular, the long acting GLP-2 analogs are considered to have “intestinotrophic” activity if, when assessed in the murine model exemplified herein, the analog mediates an increase in small bowel weight of at least 10%, 20%, or 50% relative to a control animal receiving vehicle alone. Intestinotrophic activity is noted most significantly in relation to the jejunum, including the distal jejunum and particularly the proximal jejunum, and is also noted in the ileum.

In one embodiment, the long acting GLP-2 analog of the present invention is presented by the following structure:

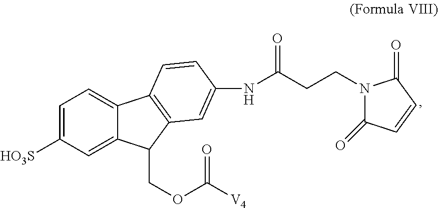

wherein V4 is the GLP-2 Analog Variant #4 having the following sequence: NH2-HGEGSFSDE(Nle)NTILDLLAARDFINWLIQTKITD-NH2 (SEQ ID NO: 4). In another embodiment, this structure is referred to as MAL-FMS-V4.

In one embodiment, the long acting GLP-2 analog of the present invention is presented by the following structure:

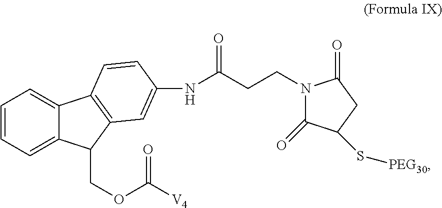

wherein V4 is the GLP-2 Analog Variant #4 having the amino acid sequence of SEQ ID NO: 4. In another embodiment, this structure is referred to as PEG30-Fmoc-V4.

In one embodiment, the long acting GLP-2 analog of the present invention is presented by the following structure:

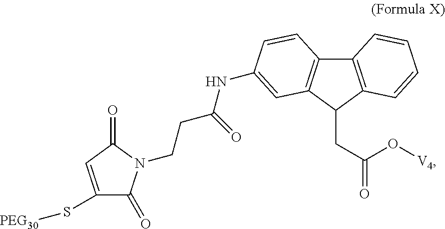

wherein V4 is the GLP-2 Analog Variant #4 having the amino acid sequence of SEQ ID NO: 4. In another embodiment, this structure is referred to as PEG30-NRF-V4.

In one embodiment, the long acting GLP-2 analog of the present invention is presented by the following structure:

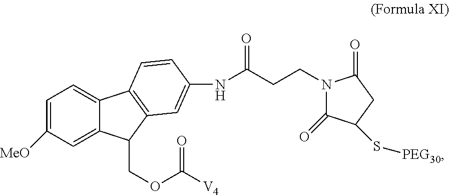

wherein V4 is the GLP-2 Analog Variant #4 having the amino acid sequence of SEQ ID NO: 4. In another embodiment, this structure is referred to as PEG30-MeOF-V4.

In one embodiment, the long acting GLP-2 analog of the present invention is presented by the following structure:

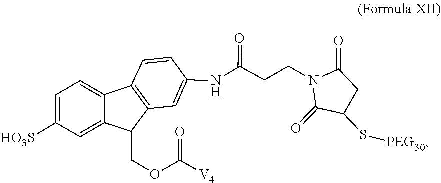

wherein V4 is the GLP-2 Analog Variant #4 having the amino acid sequence of SEQ ID NO: 4 and the linker is attached the GLP-2 Variant #4 at the Lysine position 30 of the GLP-2 analog. In another embodiment, this structure is referred to as PEG30-FMS-V4 (Lys).

In one embodiment, the long acting GLP-2 analog of the present invention is presented by the following structure:

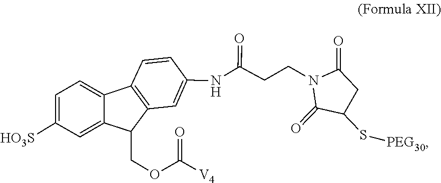

wherein V4 is the GLP-2 Analog Variant #4 having the amino acid sequence of SEQ ID NO: 4. In another embodiment, this structure is referred to as PEG30-FMS-V4.

In one embodiment, the long acting GLP-2 analog of the present invention is presented by the following structure:

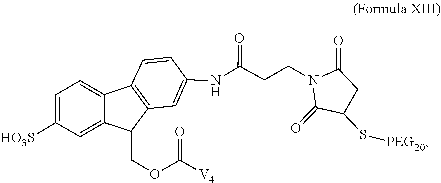

wherein V4 is the GLP-2 Analog Variant #4 having the amino acid sequence of SEQ ID NO: 4. In another embodiment, this structure is referred to as PEG20MA-FMS-V4 or PEG20-FMS-V4.

In one embodiment, the long acting GLP-2 analog of the present invention is presented by the following structure:

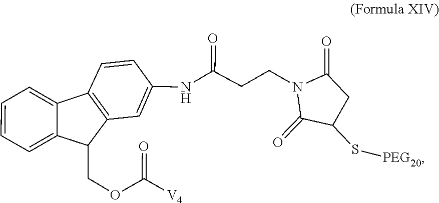

wherein V4 is the GLP-2 Analog Variant #4 having the amino acid sequence of SEQ ID NO: 4. In another embodiment, this structure is referred to as PEG20MA-Fmoc-V4 or PEG20-Fmoc-V4.

In one embodiment, the long acting GLP-2 analog of the present invention is presented by the following structure:

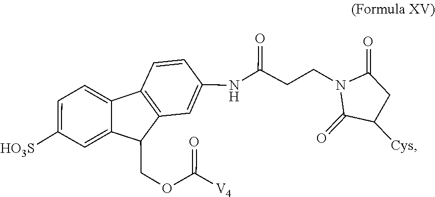

wherein V4 is the GLP-2 Analog Variant #4 having the amino acid sequence of SEQ ID NO: 4 and Cys is cysteine. In another embodiment, this structure is referred to as Cys-MAL-FMS-V4 or Cys-FMS-V4.

In one embodiment the invention relates to therapeutic and related uses of long acting GLP-2 analogs, for treating inflammation, low grade inflammation, or injury. In another embodiment, the invention relates to therapeutic and related uses of long acting GLP-2 analogs for treating inflammation, low grade inflammation, or injury by improving anti-inflammatory effects. In another embodiment the invention relates to anti-inflammatory uses of long acting GLP-2 analogs.

In one embodiment, the terms “increasing the level of” or “extending” refers to an increase of about 1-10% relative to an original, wild-type, normal or control level. In another embodiment, the increase is of about 11-20%. In another embodiment, the increase is of about 21-30%. In another embodiment, the increase is of about 31-40%. In another embodiment, the increase is of about 41-50%. In another embodiment, the increase is of about 51-60%. In another embodiment, the increase is of about 61-70%. In another embodiment, the increase is of about 71-80%. In another embodiment, the increase is of about 81-90%. In another embodiment, the increase is of about 91-95%. In another embodiment, the increase is of about 96-100%.

In another embodiment, a “pharmaceutical composition” refers to a preparation of a GLP-2 analog, a GLP-2 analog linked solely to Fmoc, MAL-Fmoc, FMS, MAL-FMS, MeOFmoc, or NRFmoc, or reversed or non-reversed PEGylated GLP-2 analogs as described herein with other chemical components such as physiologically suitable carriers and excipients. The purpose of a pharmaceutical composition is to facilitate administration of a compound to an organism. In another embodiment, a GLP-2 analog, GLP-2 analog linked solely to Fmoc, MAL-Fmoc, FMS, MAL-FMS, MeOFmoc, or NRFmoc or reverse or non-reverse pegylated GLP-2 analog is accountable for the biological effect.

In another embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979)).

For therapeutic use, the chosen GLP-2 analog, GLP-2 analog linked solely to Fmoc, MAL-Fmoc, FMS, MAL-FMS, MeOFmoc, or NRFmoc, or reversibly or non-reversibly PEGylated GLP-2 analog is formulated with a carrier that is pharmaceutically acceptable and is appropriate for delivering the peptide by the chosen route of administration. Suitable pharmaceutically acceptable carriers are those used conventionally with peptide-based drugs, such as diluents, excipients and the like. Reference may be made to “Remington's Pharmaceutical Sciences”, 17th Ed., Mack Publishing Company, Easton, Pa., 1985, for guidance on drug formulations generally. In one embodiment of the invention, the compounds are formulated for administration by infusion, e.g., when used as liquid nutritional supplements for patients on total parenteral nutrition therapy, or by injection, e.g., subcutaneously, intramuscularly or intravenously, and are accordingly utilized as aqueous solutions in sterile and pyrogen-free form and optionally buffered to physiologically tolerable pH, e.g., a slightly acidic or physiological pH. Thus, the compounds may be administered in a vehicle such as distilled water or, more desirably, in saline, phosphate buffered saline or 5% dextrose solution. Water solubility of the GLP-2 analog, GLP-2 analog linked solely to Fmoc MAL-Fmoc, FMS, MAL-FMS, MeOFmoc, or NRFmoc, or reversibly or non-reversibly PEGylated GLP-2 may be enhanced, if desired, by incorporating a solubility enhancer, such as acetic acid.

In another embodiment, “excipient” refers to an inert substance added to a pharmaceutical composition to further facilitate administration of a long-acting GLP-2 analog. In one embodiment, excipients include calcium carbonate, calcium phosphate, various sugars and types of starch, cellulose derivatives, gelatin, vegetable oils, polysorbate 20, polysorbate 80 and polyethylene glycols.

In a further aspect, the invention provides a method for promoting growth of small bowel tissue in a patient in need thereof, comprising the step of delivering to the patient an intestinotrophic amount of a GLP-2 analog, GLP-2 analog linked solely to Fmoc, MAL-Fmoc, FMS, MAL-FMS, MeOFmoc, or NRFmoc, or reversibly or non-reversibly PEGylated GLP-2 analog of the present invention.

In general, patients who would benefit from increased small intestinal mass and consequent increased small bowel mucosal function are candidates for treatment with GLP-2 analogs, GLP-2 analogs linked solely to Fmoc, MAL-Fmoc, FMS, MAL-FMS, MeOFmoc, or NRFmoc or reversibly or non-reversibly PEGylated GLP-2 analogs. Particular conditions that may be treated with GLP-2 analogs, GLP-2 analogs linked solely to Fmoc, MAL-Fmoc, FMS, MAL-FMS, MeOFmoc, or NRFmoc or reversibly or non-reversibly PEGylated GLP-2 analog include the various forms of sprue including celiac sprue which results from a toxic reaction to α-gliadin from heat, and is marked by a tremendous loss of villi of the small bowel; tropical sprue which results from infection and is marked by partial flattening of the villi; hypogammaglobulinemic sprue which is observed commonly in patients with common variable immunodeficiency or hypogammaglobulinemia and is marked by significant decrease in villus height. The therapeutic efficacy of the GLP-2 analog, GLP-2 analog linked solely to Fmoc, MAL-Fmoc, FMS, MAL-FMS, MeOFmoc, or NRFmoc or reversibly or non-reversibly PEGylated GLP-2 analog treatment may be monitored by enteric biopsy to examine the villus morphology, by biochemical assessment of nutrient absorption, by patient weight gain, or by amelioration of the symptoms associated with these conditions. Other conditions that may be treated with GLP-2 analog, GLP-2 analog linked solely to Fmoc, MAL-Fmoc, FMS, MAL-FMS, MeOFmoc, or NRFmoc or reversibly or non-reversibly PEGylated GLP-2 analog, or for which GLP-2 analog, GLP-2 analog linked solely to Fmoc, MAL-Fmoc, FMS, MAL-FMS, MeOFmoc, or NRFmoc or reversibly or non-reversibly PEGylated GLP-2 analog may be useful prophylactically, include radiation enteritis, infectious or post-infectious enteritis, regional enteritis (Crohn's disease), small intestinal damage due to toxic or other chemotherapeutic agents, intestinal complications or damage due to surgical procedure and patients with short bowel syndrome.

Chemotherapy (CT) and radiation therapy (RT) for treatment of cancers target rapidly dividing cells. Since the cells of intestinal crypts (the simple tubular glands of the small intestine) are rapidly proliferating, CT/RT tends to produce intestinal mucosal damage as an adverse effect. Gastroenteritis, diarrhea, dehydration and, in some cases, bacteremia and sepsis may ensue. These side effects are severe for two reasons: They set the limit for the dose of therapy and thereby the efficacy of the treatment, and they represent a potentially life-threatening condition, which requires intensive and expensive treatment.

In one embodiment, the present invention relates to the use of a long acting GLP-2 analog described herein for the preparation of a medicament for the treatment of a bowel disease, small bowel syndrome, inflammatory bowel syndrome, colitis including collagen colitis, radiation colitis, ulcerative colitis chronic radiation enteritis, non-tropical (gluten intolerance) and tropical sprue, Coeliac disease (gluten sensitive enteropathy), damaged tissue after vascular obstruction or trauma, diarrhea e.g. tourist diarrhea and post-infective diarrhea, chronic bowel dysfunction, dehydration, bacteremia, sepsis, anorexia nervosa, damaged tissue after chemotherapy e.g. chemotherapy-induced intestinal mucositis, premature infants incl. intestinal failure in premature infants, preborn infants incl. intestinal failure in preborn infants, schleroderma, gastritis including atrophic gastritis, postantrectomy atrophic gastritis and Helicobacter pylori gastritis, pancreatitis, general septic shock ulcers, enteritis, cul-de-sac, lymphatic obstruction, vascular disease and graft-versus-host, healing after surgical procedures, post radiation atrophy and chemotherapy, weight loss in Parkinson's Disease, intestinal adaptation after surgical procedure, parenteral nutrition-induced mucosal atrophy, e.g. total parenteral nutrition (TPN)-induced mucosal atrophy, and bone-related disorders including osteoporosis, hypercalcemia of malignancy, osteopenia due to bone metastases, periodontal disease, hyperparathyroidism, periarticular erosions in rheumatoid arthritis, Paget's disease, osteodystrophy, myositis ossificans, Bechterew's disease, malignant hypercalcemia, osteolytic lesions produced by bone metastasis, bone loss due to immobilization, bone loss due to sex steroid hormone deficiency, bone abnormalities due to steroid hormone treatment, bone abnormalities caused by cancer therapeutics, osteomalacia, Bechet's disease, osteomalacia, hyperostosis, osteopetrosis, metastatic bone disease, immobilization-induced osteopenia, or glucocorticoid-induced osteoporosis.

In one embodiment, the present invention relates to the use of a long acting GLP-2 analog described herein for the preparation of a medicament for the treatment of acid-induced intestinal injury, arginine deficiency, autoimmune diseases, bacterial peritonitis, bowel ischemia, bowel trauma, burn-induced intestinal damage, catabolic illness, celiac disease, chemotherapy-associated bacteremia, chemotherapy-induced enteritis, decreased gastrointestinal motility, diabetes, diarrheal diseases, fat malabsorption, febrile neutropenia, food allergies, gastric ulcers, gastrointestinal barrier disorders, gastrointestinal injury, hypoglycemia, idiopathic hypospermia, inflammatory bowel disease, intestinal failure, intestinal insufficiency, irritable bowel syndrome, ischemia, malnutrition, mesenteric ischemia, mucositis, necrotizing enterocolitis, necrotizing pancreatitis, neonatal feeding intolerance, neonatal nutritional insufficiency, NSAID-induced gastrointestinal damage, nutritional insufficiency, obesity, pouchitis, radiation-induced enteritis, radiation-induced injury to the intestines, steatorrhea, stroke, or total parenteral nutrition damage to gastrointestinal tract.

In another embodiment, the particular conditions that may be treated with the long acting GLP-2 analogs include the various forms of inflammatory diseases of the stomach or esophagus, as well as patients who have undergone partial or sub-total resection of the upper gastrointestinal tract. A non-exhaustive list of conditions of the upper gastrointestinal tract including the stomach and esophagus, that may be treated by the present long acting GLP-2 analogs or mixtures thereof, comprises disorders of the stomach like acute gastritis, acute hemorrhagic gastritis, acute stress gastritis, viral gastritis, parasitic gastritis, fungal gastritis, gastropathy (acute), hemorrhagic gastropathy, acute Helicobacter pylori gastritis, type A, B or C gastritis, hypersecretory gastritis, non specific gastritis secondary to Helicobacter pylori, Helicobacter pylori-associated gastritis, chemical gastritis, reactive gastritis, reflux gastritis, bile gastritis, metaplastic atrophic gastritis and environmental metaplastic atrophic gastritis, idiopathic pangastritis, diffuse corporal gastritis, autoimmune chronic gastritis and autoimmune-associated gastritis, bacterial gastritis other than Helicobacter pylori (Gastrospirillum hominis, phlegmonous, mycobacterial, syphiltic), postantrectomy atrophic gastritis, eosinophilic gastritis, and any other acute infectious gastritis; Crohn's disease, sarcoidosis, isolated granulomatous gastritis, lymphocylic gastritis, Menetriere's disease, etc., and disorders of the esophagus like infectious esophagitis from fungi like Candida species (esp. albicans), Aspargillus sp., Histoplasma capsulatum, Blastomyces dermatitides, or from viruses like herpes simplex virus (type 1), cytomegalovirus, Varicella-zoster virus, or from bacteria like Mycobacterium tuberculosis, Actinomyces israelii, Streptococcus viridans, Lactobacillus acidophilus, and Treponema pallidum. Other disorders of the esophagus include, without limitation, non-infectious esophagitis, acid reflux, bile reflux, chemical injury (caused by medicines, toxins, acids, alkali etc.), sarcoidosis, Crohn's disease, Behcet's disease, Graft-versus-host disease, AJDS Related Infections (Cryptosporidium sp., Microsporidium sp., Isospora beill, Glardia lamblia, Salmonella sp., Shigella sp., Campylobacter sp., Mycobacterium tuberculosis, Mycobacterium avium complex, Clostridium difficile, Cytomeglavorius and Herpes simplex.

In another embodiment, other diseases or conditions that can be treated with the long acting GLP-2 analogs include abnormalities in the small intestinal tract mucosa, which include ulcers and inflammatory disorders; congenital or acquired digestion and absorption disorders including malabsorption syndromes; and diseases and conditions caused by loss of small intestine mucosal function particularly in patients undergoing extended parenteral feeding or who, as a result of surgery, have undergone resection of the small intestine and suffer from short-gut syndrome and cul-de-sac syndrome, hi general, patients who would benefit from either increased small intestinal mass and consequent increased small intestine mucosal function are candidates for treatment with long acting GLP-2 analogs. Particular conditions that may be treated with the present long acting GLP-2 analogs include the various forms of sprue including celiac sprue which results from a toxic reaction to gliadin from wheat, and is marked by a tremendous loss of villi of the small intestine; tropical sprue which results from infection and is marked by partial flattening of the villi; hypogammaglobulinemic sprue which is observed commonly in patients with common variable immunodeficiency or hypogammaglobulinemia and is marked by significant decrease in villus height. Other conditions that may be treated with the present long acting GLP-2 analogs, or for which they may be useful prophylactically, include radiation enteritis, infectious or post-infectious enteritis, regional enteritis (Crohn's disease), small intestinal damage due to toxic or other chemotherapeutic agents, and patients with short bowel syndrome.

The therapeutic dosing and regimen most appropriate for patient treatment will of course vary with the disease or condition to be treated, and according to the patient's weight and other parameters. The results presented hereinbelow demonstrate that a dose of GLP-2 peptide, that presumably equivalent to about 15 mg/kg (or less) administered twice daily over 10 days can generate very significant increases in small bowel mass in rats. It is expected that much smaller doses, e.g., in the g/kg range, and shorter or longer duration or frequency of treatment, will also produce therapeutically useful results, i.e., a statistically significant increase particularly in small bowel mass or any other relevant clinically meaningful outcome. Also, it is anticipated that the therapeutic regimen will include the administration of maintenance doses appropriate for reversing tissue regression that occurs following cessation of initial treatment. The dosage sizes and dosing regimen most appropriate for human use are guided by the results herein presented and can be confirmed in properly designed clinical trials.

In one embodiment, a typical human dose of a the long acting GLP-2 analog (specific peptide content) would be from about 10 μg/kg body weight/day to about 10 mg/kg/day, or from about 50 μg/kg/day to about 5 mg/kg/day, or about 100 μg/kg/day to 1 mg/kg/day. In another embodiment, a typical dose of the long acting GLP-2 analog would be from about 100 ng/kg body weight/day to 1 mg/kg/day, or 1 μg/kg/day to 500 μg/kg/day, or 1 μg/kg/day to 100 g/kg/day.

In another embodiment, pharmaceutical composition comprising a long acting GLP-2 analog of this invention is administered once a day. In another embodiment, a pharmaceutical composition comprising a long acting GLP-2 analog of this invention is administered once every 36 hours. In another embodiment, pharmaceutical composition comprising a long acting GLP-2 analog of this invention is administered once every 48 hours. In another embodiment, pharmaceutical composition comprising a long acting GLP-2 analog of this invention is administered once every 60 hours. In another embodiment, a pharmaceutical composition comprising a long acting GLP-2 analog of this invention is administered once every 72 hours. In another embodiment, a pharmaceutical composition comprising a long acting GLP-2 analog of this invention is administered once every 84 hours. In another embodiment, a pharmaceutical composition comprising a long acting GLP-2 analog of this invention is administered once every 96 hours. In another embodiment, a pharmaceutical composition comprising a long acting GLP-2 analog of this invention is administered once every 5 days. In another embodiment, a pharmaceutical composition comprising a long acting GLP-2 analog of this invention is administered once every 6 days. In another embodiment, a pharmaceutical composition comprising a long acting GLP-2 analog of this invention is administered once every 7 days. In another embodiment, a pharmaceutical composition comprising a long acting GLP-2 analog of this invention is administered once every 8-10 days. In another embodiment, a pharmaceutical composition comprising a long acting GLP-2 analog of this invention is administered once every 10-12 days. In another embodiment, a pharmaceutical composition comprising a long acting GLP-2 analog of this invention is administered once every 12-15 days. In another embodiment, a pharmaceutical composition comprising a long acting GLP-2 analog of this invention is administered once every 15-25 days. In another embodiment, a pharmaceutical composition comprising a long acting GLP-2 analog of this invention is administered twice a week. In another embodiment, a pharmaceutical composition comprising a long acting GLP-2 analog of this invention is administered once weekly. In another embodiment, a pharmaceutical composition comprising a long acting GLP-2 analog of this invention is administered once every other week.

In another embodiment, a typical human dose of a long acting GLP-2 analog would be from about 10 μg/kg body weight/twice weekly to about 10 mg/kg/twice weekly, or from about 50 μg/kg/twice weekly to about 5 mg/kg/twice weekly, or about 100 μg/kg/twice weekly to 1 mg/kg/twice weekly. In another embodiment, a typical dose of the long acting GLP-2 analog would be from about 100 ng/kg body weight/twice weekly to 1 mg/kg/twice weekly, or 1 μg/kg/twice weekly to 500 μg/kg/twice weekly, or 1 μg/kg/twice weekly to 100 g/kg/twice weekly.

In one embodiment, a typical human dose of a long acting GLP-2 analog would be from about 10 μg/kg body weight/week to about 10 mg/kg/week, or from about 50 μg/kg/week to about 5 mg/kg/week, or about 100 μg/kg/week to 1 mg/kg/week. In another embodiment, a typical dose of the long acting GLP-2 analog would be from about 100 ng/kg body weight/week to 1 mg/kg/week, or 1 μg/kg/week to 500 μg/kg/week, or 1 μg/kg/week to 100 g/kg/week.

In one embodiment, a typical human dose of a long acting GLP-2 analog would be from about 10 μg/kg body weight/every other week to about 10 mg/kg/every other week, or from about 50 μg/kg/every other week to about 5 mg/kg/every other week, or about 100 μg/kg/every other week to 1 mg/kg/every other week. In another embodiment, a typical dose of the long acting GLP-2 analog would be from about 100 ng/kg body weight/every other week to 1 mg/kg/every other week, or 1 μg/kg/every other week to 500 μg/kg/every other week, or 1 μg/kg/every other week to 100 g/kg/every other week.

In one embodiment, a typical human dose of a long acting GLP-2 analog would be about 50 μg/kg/twice weekly. In one embodiment, a typical human dose of a long acting GLP-2 analog would be about 50 μg/kg/week. In one embodiment, a typical human dose of a long acting GLP-2 analog would be about 50 μg/kg/every other week.

In another embodiment, a conjugate or a peptide coupled to a linker said FMS, MAL-FMS, Fmoc, MAL-Fmoc, or MeOFmoc or combination of them of this invention is administered by an intramuscular (IM) injection, subcutaneous (SC) injection, or intravenous (IV) injection once a week.

In another embodiment, suitable routes of administration of the peptide of the present invention, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In another embodiment, the GLP-2 analogs, GLP-2 analogs linked solely to Fmoc MAL-Fmoc, FMS, MAL-FMS, or MeOFmoc, or reversibly or non-reversibly PEGylated GLP-2 analogs can be provided to the individual per se. In one embodiment, the present invention can be provided to the individual as part of a pharmaceutical composition where it is mixed with a pharmaceutically acceptable carrier.

In one embodiment, the following synthetic schemes outlined in Table 17 are followed to produce the conjugates listed at the top of each column. In another embodiment, the synthetic schemes in Table 17 can be used for any GLP-2 analogs or variants.

TABLE 17

| PEGylated | | PEGylated | |
|---|---|---|---|
| FMOC-GLP-2 | FMOC-GLP-2 | FMS-GLP-2 | FMS-GLP-2 |
| Analog | Analog | Analog | Analog |
| Conjugate | Conjugated | Conjugated | Conjugate |
| STAGE 1 | STAGE 1 | STAGE 1 | STAGE 1 |
| Solid phase peptide | Solid phase peptide | Solid phase peptide | Solid phase peptide |
| synthesis (SPPS) | synthesis (SPPS) | synthesis (SPPS) of | synthesis (SPPS) of |
| of the GLP-2 | of the GLP-2 | the GLP-2 | the GLP-2 |
| variant peptide | variant peptide | variant peptide | variant peptide |
| | | STAGE 2 | STAGE 2 |
| | | Sulfonation of | Sulfonation of |
| | | Fmoc linker | Fmoc linker |
| | | (pre-linker) to | (pre-linker) to |
| | | FMS linker | FMS linker |
| STAGE 2 | STAGE 2 | STAGE 3 | STAGE 3 |
| Coupling of the | Coupling of the | Coupling of the | Coupling of the |
| Linker to GLP-2 | Linker to GLP-2 | Linker to GLP-2 | Linker to GLP-2 |
| variant on the | variant on the | variant on the | variant on the |
| SPPS resin | SPPS resin | SPPS resin | SPPS resin |
| | STAGE 3 | | STAGE 4 |
| | Reduction of the | | Reduction of the |
| | maleimide group | | maleimide group |
| | or coupling to a | | or coupling to a |
| | thiol containing | | thiol containing |
| | molecule | | molecule |
| STAGE 3 | STAGE 4 | STAGE 4 | STAGE 5 |
| Cleavage of | Cleavage of | Cleavage of | Cleavage of |
| peptide from | peptide from | peptide from | peptide from |
| the resin | the resin | the resin | the resin |
| STAGE 4 | STAGE 5 | STAGE 5 | STAGE 6 |
| Purification of | Purification of | Purification of | Purification of |
| MAL-Fmoc- | (+/−molecule-S-)FMOC- | MAL-FMS- | (+/−molecule-S-)FMS- |
| (GLP-2 Variant) | PEPTIDE-(GLP-2 | (GLP-2 Variant) | PEPTIDE-(GLP-2 |
| | Variant) | | Variant) |
| STAGE 5 | | STAGE 6 | |
| PEGylation and | | PEGylation and | |
| purification | | purification | |
| STAGE 6 | STAGE 6 | STAGE 7 | STAGE 7 |
| Salt exchanging | Salt exchanging | Salt exchanging | Salt exchanging |
| and microfiltration | and microfiltration | and microfiltration | and microfiltration |
| STAGE 7 | STAGE 7 | STAGE 8 | STAGE 8 |
| Final lyophilization | Final lyophilization | Final lyophilization | Final lyophilization |

### EXAMPLES

### Example 1: Synthesis of Mal-Fmoc-Nhs, Mal-Fms-Nhs, Mal-Linker-Osu and Manipulation of the Mal-Linker-Osu

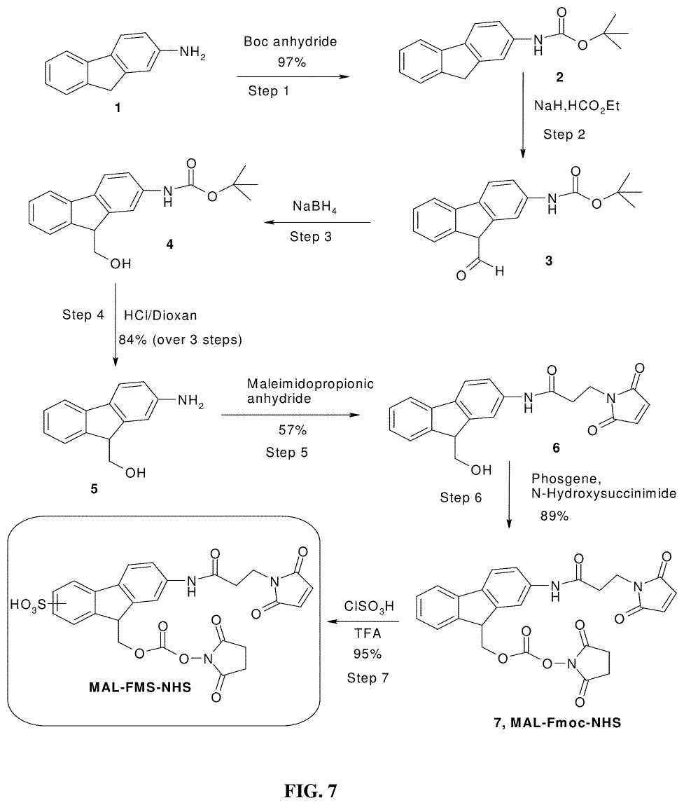

The peptide was synthesized by the solid phase method employing the Fmoc-strategy throughout the peptide chain assembly (Almac Sciences, Scotland) and shown in FIG. 7.

The peptide sequence was assembled using the following steps:

### 1. Capping

The resin was capped using 0.5M acetic anhydride (Fluka) solution in DMF (Rathburn).

### 2. Deprotection

Fmoc-protecting group was removed from the growing peptide chain using 20% v/v piperidine (Rathburn) solution in DMF (Rathburn).

### 3. Amino Acid Coupling

0.5M Amino acid (Novabiochem) solution in DMF (Rathburn) was activated using 1M HOBt (Carbosynth) solution in DMF (Rathburn) and 1M DIC (Carbosynth) solution in DMF (Rathburn). 4 equivalents of each amino acid were used per coupling.

The crude peptide is cleaved from the resin and protecting groups removed by stirring in a cocktail of Triisopropylsilane (Fluka), water, dimethylsulphide (Aldrich), ammonium iodide (Aldrich) and TFA (Applied Biosystems) for 4 hours. The crude peptide is collected by precipitation from cold diethyl ether.

### Peptide Purification

Crude peptide was dissolved in acetonitrile (Rathburn)/water (MilliQ) (5:95) and loaded onto the preparative HPLC column. The chromatographic parameters are as follows: Column: Phenomenex Luna C18 250 mm×30, 15 μm, 300 A

Mobile Phase A: water+0.1% v/v TFA (Applied Biosystems)
Mobile Phase B: acetonitrile (Rathburn)+0.1% v/v TFA (Applied Biosystems)

### UV Detection: 214 or 220 nm

Gradient: 25% B to 31% B over 4 column volumes
Flow rate 43 mL/min

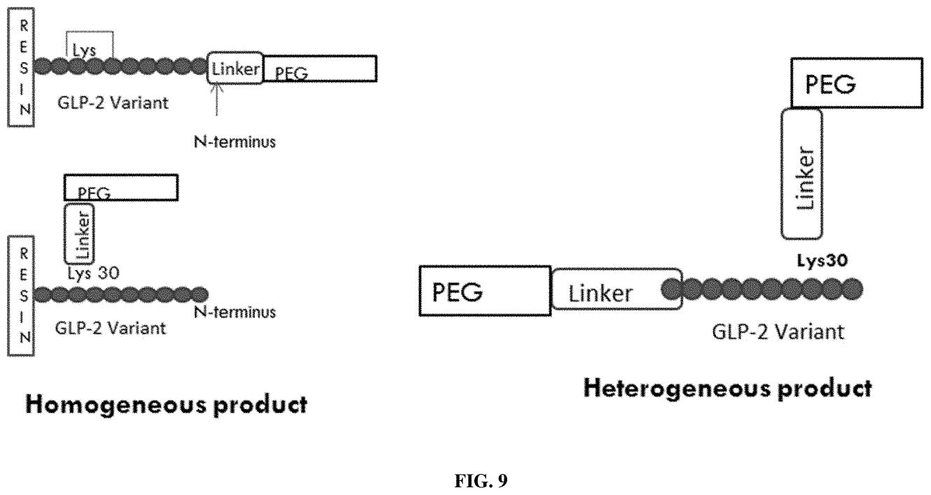

Stage 2—Linker Synthesis (FIG. 9)

The synthesis of compounds 2-5 (FIG. 9) is based on the procedures described by Albericio et al. in Synthetic Communication, 2001, 31(2), 225-232.

### 2-(Boc-amino)fluorene (2):

2-Aminofluorene (18 g, 99 mmol) was suspended in a mixture of dioxane:water (2:1) (200 ml) and 2N NaOH (60 ml) in an ice bath with magnetic stirring. Boc₂O (109 mmol, 1.1 eq) was then added and stirring continued at RT. The reaction was monitored by TLC (Rf=0.5, Hex./Ethyl Acetate 2:1) and the pH maintained between 9-10 by addition of 2N NaOH. At reaction completion, the suspension was acidified with 1M KHSO4 to pH=3. The solid was filtered and washed with cold water (50 ml), dioxane-water (2:1) and then azeotroped with toluene twice before using it in the next step.

### 9-Formyl-2-(Boc-amino)fluorene (3):

In a 3 necked RBF, NaH (60% in oil; 330 mmol, 3.3 eq) was suspended in dry THF (50 ml), a solution of -(Boc-amino)fluorine described in step 2 (28 g; 100 mmol) in dry THF (230 ml) was added dropwise over 20 minutes. A thick yellow slurry was observed and the mixture stirred for 10 minutes at RT under nitrogen. Ethyl formate (20.1 ml, 250 mmol, 2.5 eq) was added dropwise (Caution: gas evolution). The slurry turned to a pale brown solution. The solution was stirred for 20 minutes. The reaction was monitored by TLC (Rf=0.5, Hex./Ethyl acetate 1:1) and when only traces of starting material was observed, it was quenched with iced water (300 ml). The mixture was evaporated under reduce pressure until most of the THF has been removed. The resulting mixture was treated with acetic acid to pH=5. The white precipitate obtained was dissolved in ethyl acetate and the organic layer separated. The aqueous layer was extracted with ethyl acetate and all the organic layer combined and washed with saturated sodium bicarbonate, brine and dried over MgSO₄. After filtration and solvent removal a yellow solid was obtained. This material was used in the next step.

### 9-Hydroxymethyl-2-(Boc-amino)fluorene (4):

Compound 3 from above was suspended in MeOH (200 ml) and sodium borohydride was added portion wise over 15 minutes. The mixture was stirred for 30 minutes (caution: exothermic reaction and gas evolution). The reaction was monitored by TLC (Rf=0.5, Hex./EtOAc 1:1) and was completed. Water (500 ml) was added and the pH adjusted to 5 with acetic acid. The work up involved extraction twice with ethyl acetate, washing the combined organic layers with sodium bicarbonate and brine, drying over MgSO₄, filtration and concentration to dryness. The crude obtained was purified by flask chromatography using Heptane/EtOAc (3:1) to give a yellow foam (36 g, 97.5% purity, traces of ethyl acetate and diethyl ether observed in the 1H-NMR).

### MAL-Fmoc-NHS (7):

To a clean dry 500 ml RBF with overhead agitation was charged triphosgene (1.58 g, 0.35 eq.) in dry THF (55 ml) to form a solution at ambient. This was cooled to 0° C. with an ice/water bath and a solution of NHS (0.67 g, 0.38 eq) in dry THF (19 ml) added dropwise over 10 minutes under nitrogen at 0° C. The resultant solution was stirred for 30 minutes. A further portion of NHS (1.34 g, 0.77 eq) in dry THF (36 ml) was added dropwise at 0° C. over 10 minutes and stirred for 15 minutes.

Compound 6 (5.5 g, 1 eq), dry THF (55 ml) and pyridine (3.07 ml, 2.5 eq) were stirred together to form a suspension. This was added to the NHS solution in portions a 0-5° C. and then allowed to go to RT by removing the ice bath.

After 20 hours the reaction was stop (starting material still present, if the reaction is pushed to completion a dimmer impurity has been observed).

The reaction mixture was filtered and to the filtrate, 4% brine (200 ml) and EtOAc (200 ml) were added. After separation, the organic layer was washed with 5% citric acid (220 ml) and water (220 ml). The organic layer was then concentrated to give 7.67 g of crude MAL-Fmoc-NHS. The material was purified by column chromatography using a gradient cyclohexane/EtOAc 70:30 to 40:60. The fractions containing product were concentrated under vacuum to give 3.47 g (45%) of MAL-Fmoc-NHS.

### MAL-FMS-NHS

To a solution of MAL-Fmoc-NHS (100 mg, 0.2 mmol) in trifluoroacetic acid (10 ml), chlorosulfonic acid (0.5 ml) was added. After 15 minutes, ice-cold diethyl ether (90 ml) was added and the product precipitated. The material was collected by centrifugation, washed with diethyl ether and dried under vacuum. 41.3 mg (35%) of beige solid was obtained.

### Example 2: Pharmacokinetics and Pharmacology Effect of Reversible Pegylation Technology with Teduglutide in SD Rats

The reversible PEGylation technology was applied to the available commercial GLP-2 analog teduglutide (GLP-2-Gly2) in order to evaluate its longevity and the efficacy in SD rats. The following PEG weights and linkers were conjugated with teduglutide: PEG30-FMS-(GLP-2-Gly2), PEG40-FMS-(GLP-2-Gly2), PEG40-Fmoc-(GLP-2-Gly2), PEGBranched40-FMS-(GLP-2-Gly2), PEGBranched30-FMS-(GLP-2-Gly2). In addition, the following non-reversible PEGylation was conjugated to GLP-2-Gly2: PEG40-EMCS-(GLP-2-Gly2).

In the study, the conjugates consist of a heterogenous product containing a mixture of PEG attached to GLP-2 via a linker at the N terminus and PEG attached to GLP-2 via a linker at the lysine residue on position number thirty (Lys30) of the GLP-2. The branched PEGylation was represented by (PEG)2-R—SH, in which R is the GLP-2 conjugate.

In the pharmacology study, the conjugates were injected subcutaneously twice, on days 1 and 3 at 1 mg/kg (peptide dose) and intestine weight was performed on day 6. In addition to the PEGylated conjugates, GLP-2-Gly2 was injected at the same dose and regimen for comparison. The average of the intestine weight of each group was compared to that of the vehicle group.

TABLE 1
Intestine weight study results of different conjugated GLP-2

| | | Peptide | Material | Average | % CV of | Increase of |
|---|---|---|---|---|---|---|
| | | dose | dose | Intestine | Intestine weight | intestine weight |
| Group | Material | mg/kg | mg/kg^{a} | weight (gr) | between animals | over vehicle (%) |
| (A) | GLP-2-Gly2 | 1 | 1 | 5.7 | 14.3 | 5 |
| | (Teduglutide) | (2 injections) | | | | |
| (B) | PEG(30)-FMS- | | 9 | 9.5 | 6.2 | 73 |
| | GLP-2-Gly2 | | | | | |
| (C) | PEG(40)-FMS- | | 13 | 9.3 | 9.0 | 70 |
| | GLP-2-Gly2 | | | | | |
| (D) | PEG(40)-Fmoc- | | 13 | 9.4 | 10.6 | 72 |
| | GLP-2-Gly2 | | | | | |
| (E) | PEG(40)-EMCS- | | 14 | 6.6 | 6.7 | 19 |
| | GLP-2-Gly2 | | | | | |
| (F) | PEG(Branch40)- | | 13 | 8.1 | 9.7 | 48 |
| | EMS- GLP-2-Gly2 | | | | | |
| (G) | PEG(Branch20)- | | 7 | 7.8 | 9.5 | 42 |
| | EMS- GLP-2-Gly2 | | | | | |
| (H) | Vehicle | N/A | N/A | 5.5 | 11.8 | N/A |
| *Average of | GLP-2-Gly2 | 2.5 | 2 | — | — | 25 |
| five studies | (Teduglutide) | (5 injections) | | | | |

^{a}The material dose was calculated based on its peptide content and purity

The average intestine weight, percent coefficient of variability (% CV), and the increase of intestine weight percentage compared to control are presented in Table 1. The two injections regimen of teduglutide at 1 mg/kg was not effective and did not increase intestine weight compared to vehicle. However, the usage of the reversible PEGylation technology with teduglutide significantly improved the efficacy on the rat's intestine weight (P<0.001) with intestine weight increase between 42-73%. The non-reversible conjugate showed significant increase in intestine weight compared to the vehicle group (P<0.01), however, was not significantly different compared to the effect that was achieved using teduglutide alone. The three linear PEGylated conjugates (PEG30-FMS-(GLP-2-Gly2), PEG40-FMS-(GLP-2-Gly2) and PEG40-Fmoc-(GLP-2-Gly2)) showed the most significant increase in intestine weight. In addition, the several studies conducted with teduglutide have shown that daily injections for five days at 2.5 mg/kg resulted with an average of 25% increase in intestine weight. Therefore, not only did the reversible PEGylation of GLP-2-Gly2 result in better efficacy, it was also achieved using lower peptide doses and injection frequency.

In order to evaluate the potential of the reversible PEGylation technology to extend the half life of teduglutide, the conjugates above were injected once at 2 mg/kg (peptide dose), except for PEG40-Fmoc-(GLP-2-Gly2), which was injected at 1 mg/kg (due to lack of material) and PEGBranch40-FMS-(GLP-2-Gly2). Plasma samples were collected at pre-dose, 0.5, 2, 4, 8, 12, 24, 48, 72, 96, 168, 216, and 240 hours post dose. The levels of conjugates and free teduglutide were measured using commercial GLP2 ELISA kit.

TABLE 2
Half-life (t_{1/2}) results of different conjugated GLP-2-Gly2

| | | Peptide | Material | |
|---|---|---|---|---|
| | | dose | dose | Half-Life |
| Group | Material | mg/kg | mg/kg | (t_{1/2}, hr) |
| (A) | GLP-2-Gly2 (Teduglutide) | 2 | 1 | 0.9 |
| (B) | PEG(30)-FMS-(GLP-2-Gly2) | | 9 | 13.1 |
| (C) | PEG(40)-FMS-(GLP-2-Gly2) | | 13 | 15.8 |
| (D) | PEG(40)-Fmoc-(GLP-2-Gly2) | 1 | 13 | 24.3 |
| (E) | PEG(40)-EMCS-(GLP-2-Gly2) | 2 | 14 | 27.5 |
| (F) | PEG(Branch20)-FMS- | | 7 | 15.5 |
| | (GLP-2-Gly2) | | | |

The t_{1/2} values are presented in Table 2. Teduglutide was detectable up to 4 hours with short half-life of 0.9 hours, while the reversible conjugated teduglutide were still visible at 168 hr (day 7) with significant increase in half-life (between 13.1 to 24.3). The non-reversible teduglutide showed the longest half-life (27.5 hr) due to the constant PEGylation of the peptide.

In a different study, several of the PEGylated teduglutide were injected as a single dose at 1 mg/kg and the intestine weight performed on day 6. The results are presented in Table 3.

TABLE 3
Intestine weight study results of different
conjugated GLP-2-Gly2 on day 6

| | | Peptide | Material | Increase of |
|---|---|---|---|---|
| | | dose | dose | intestine weight |
| Group | Material | mg/kg | mg/kg^{a} | over vehicle (%)^{a} |
| (A) | PEG(30)-FMS- | 1 | 9 | 34 |
| | (GLP-2-Gly2) | (Single | | |
| (B) | PEG(40)-FMS- | dose) | 13 | 27 |
| | (GLP-2-Gly2) | | | |
| (C) | PEG(40)-Fmoc- | | 13 | 31 |
| | (GLP-2-Gly2) | | | |
| (D) | PEG(Branch40)- | | 13 | 22 |
| | FMS-(GLP-2-Gly2) | | | |

^{a}Average of two studies

### Example 3: Construction of Mutated/Modified GLP-2 Peptide Variants

The construction of GLP-2 analogues with enhanced pharmacokinetic/pharmacodynamic (PK/PD) profiles was undertaken. The constructed GLP-2 analogues were assessed based on (1) their physio-chemical properties and their chemistry, manufacturing and control (CMC) considerations and (2) their biological performances. Thus, point mutations were induced to the GLP-2 native sequence. The sequence mutations, and their specific combinations, aimed to shed light on the peptide potential with respect to CMC characteristics and biological performances, are presented in Table 4. Variants 1 and 10 in Table 4 served as control for both stability and biological performances and these variants are currently in clinical development.

The improved GLP-2 analogues may also be conjugated with PEG and a linker, or linker alone, or a linker with a reduced maleimide group as described throughout the application to receive a superiority of longevity and activity.

TABLE 4
Mutated GLP-2 Variants

| Variant # | AA Sequence | Mutations | Mw | PI |
|---|---|---|---|---|
| 1 | NH2-HG²DGSFSDENle¹⁰(D- | Position 2 Gly, 10 Nle, 11 D-Phe, | 3765 | 4.26 |
| | F)¹¹TILDL¹⁶LAAR²⁰DFINWLIQTKITD-NH2 | 16 Leu, C terminus CONH2 | | |
| 2 | NH2-HG²E³GSFSDE(Nle)¹⁰(D- | Position 2 Gly, 3 Glu, 10 Nle, 11 | 3780 | 4.35 |
| | F)¹¹TILDN¹⁶LAARDFINWLIQTKITD-NH2 | D-Phe, C Terminus CONH2 | | |
| 3 | NH2-HG²E³GSFSDE(Nle)¹⁰(D- | Position 2 Gly, 3 Glu, 10 Nle, | 3770 | 4.35 |
| | H)¹¹TILDN¹⁶LAARDFINWLIQTKITD-NH2 | 11 D-His, C Terminus CONH2 | | |
| 4 | NH2-HG²E³GSFSDE(Nle)¹⁰N¹¹TILDL¹⁶LAARDFINW | Position 2 Gly, 3 Glu, 10 Nle, | 3746 | 4.35 |
| | LIQTKITD-NH2 | 16 Leu, C terminus CONH2 | | |
| 5 | NH2-HG²E³GSFSDE(Nle)¹⁰N¹¹TILDY¹⁶LAARDFIN | Position 2 Gly, 3 Glu, 10 Nle, | 3796 | 4.35 |
| | WLIQTKITD-NH2 | 16 Tyr, C terminus CONH2 | | |
| 6 | NH2-HG²E³GSFSDE(Nle)¹⁰N¹¹TILDL¹⁶LAARDFINW | Position 2 Gly, 3 Glu ,10 Nle, | 3747 | 4.35 |
| | LIQTKITD-COOH | 16 Leu | | |
| 7 | NH2-HG²E³GSFSDE(Nle)¹⁰N¹¹TILDY¹⁶LAARDFIN | Position 2 Gly, 3 Glu, 10 Nle, | 3797 | 4.35 |
| | WLIQTKITD-COOH | 16 Tyr | | |
| 8 | NH2-HG²DGSFSDENle¹⁰(D- | Position 2 Gly, 10 Nle, 11 D-Phe, | 3766 | 4.26 |
| | F)¹¹TILDL¹⁶LAARDFINWLIQTKITD-COOH | 16 Leu | | |
| 9 | NH2-HG²E³GSFSDENle¹⁰(D- | Position 2 Gly, 3 Glu, 10 Nle, | 3779 | 4.35 |
| | F)¹¹TILDL¹⁶LAARDFINWLIQTKITD-NH2 | 11 D-Phe, 16 Leu, C terminus CONH2 | | |
| 10 | NH2-HG²E³GT⁵FSS⁸EL¹⁰A¹¹TILDA¹⁶LAAR20DFIA²⁴ | Position 2 Gly, 3 Glu, 5 Thr, 8 Ser, | 4316 | 8.84 |
| | WLIA²⁸TKITDK³⁴K³⁵K³⁶K³⁷K³⁸K³⁹- NH2 | 10 Leu, 11 Ala, 16 Ala, 24 Ala, 28 | | |
| | | Ala, 35-39 Lys, C terminus CONH2 | | |

The GLP-2 analogues were tested for stability via appearance, O.D. reading (A.280, and A.325) and RP-HPLC (purity, and peak area) following t=0, 1 day, and 48 h incubation at 37° C. Table 5 below highlights these characteristics (following 48-hour incubation at 37° C.) in sequences differing in only one mutation, and yet surprising, these GLP-2 analogues exhibited distinct characteristic with respect to CMC (Study reference 0042).

TABLE 5
CMC characteristics (following 48 h incubation at 37° C.) with respect to the AA sequence mutation

| | Solubility | Solubility | Stability | |
|---|---|---|---|---|
| Variant | via | via A. | via RP-HPLC | |
| comparison | appearance | 325 reading | main peak | CMC related Comments |
| V1 Vs. V9 | V1: opaque | V1: 0.022 | V1: −20% | The sequence Gly-Asp can undergo Asp rearrangement. |
| D Vs. E | V9: clear | V9: 0.002 | V9: −1.5% | Replacing Asp with Glu does not change the overall |
| Position 3 | | | | charge and polarity of the drug, but prevent the non- |
| | | | | desired side effect causing molecule instability |
| V2 Vs. V4 | V2: opaque | V2: 0.237 | V2: −30% | The Asn is the native amino acid at position 11 and |
| D-F Vs. N | V4: clear | V4: 0.004 | V4: −1.5% | included in V4. Although D-F replacement at the |
| Position 11 | | | | hydrophobic side of the peptide might increase peptide |
| | | | | stability, our results demonstrates better stability for |
| | | | | the Asn sequence at this position. |
| V3 Vs. V4 | V3: clear | V3: 0.008 | V3: −4% | The Asn is the native amino acid at position 11 and |
| D-His Vs. N | V4: clear | V4: 0.004 | V4: −1.5% | included in V4. Although D-H replacement at the |
| Position 11 | | | | hydrophobic side of the peptide might increase |
| | | | | peptide stability, our results demonstrates better |
| | | | | stability for the Asn sequence at this position. |
| V4 Vs. V5 | V4: clear | V4: 0.004 | Similar | None |
| L Vs. Y | V5: clear | V5: 0.019 | main peak | |
| Position 16 | | | stability | |
| | | | (~1.5%) | |
| V4 Vs. V6 | V4: clear | V4: 0.004 | Similar | Amidation of the C- terminus prevent carboxy peptidase |
| Amidated Vs. | V6: clear | V6: 0.013 | main peak | mediated degradation |
| carboxylated | | | stability | |
| C-terminus | | | (~1.5%) | |
| V5 Vs. V7 | V5: clear | V5: 0.019 | Similar | Amidation of the C- terminus prevent carboxy peptidase |
| Amidated Vs. | V7: clear | V7: 0.022 | main peak | mediated degradation |
| carboxylated | | | stability | |
| C-terminus | | | (no change | |
| | | | from time = 0) | |

Several other studies (Study references 51, 55, and 59) were performed to determine the analogues' solubility and stability in different buffer systems (Histidine and NaPi, at pHs between 6.8 to 7.5), peptide concentrations (between 1-10 mg/mL), and incubation times (up to 3 days at 37° C.) and following freeze-thaw stresses (up to three cycles). From a CMC perspective, combining purity and solubility, variants 6-7 displayed similar characteristics compared to V4, and all of the three (variants 4, 6, and 7) outperformed the rest of the peptides.

Pharmacology and pharmacokinetic studies were performed with the most promising GLP-2 variants to evaluate their efficacy and longevity, respectively. The intestine weight model in rats was conducted to evaluate efficacy, by measuring percent increase of the intestine weight of treated animals over the vehicle group. In this study, the peptides were injected twice, on days 1 and 3 at 1 mg/kg and the intestine weight was conducted on day 6. The results are summarized in Table 6.

TABLE 6
Pharmacology and Pharmacokinetics results of modified peptides

| | | Peptide dose | Peptide | Increase of | |
|---|---|---|---|---|---|
| | | for Efficacy | dose for | intestine | |
| | GLP-2 | study | PK study | weight over | Half- |
| Group | Variant# | mg/kg | mg/kg | vehicle (%) | Life (hr) |
| (A) | V3 | Two | 15 mg/kg | 4 | 1.9 |
| (B) | V4 | injections | | 89 | 17.5 |
| (C) | V5 | on days 1 & | | 10 | 6.5 |
| (D) | V6 | 3, at 1 mg/kg | | 43 | 12.1 |
| (E) | V7 | | | 12 | 5.8 |

As shown in Table 6, variants 4 and 6 showed improved efficacy with the percent intestine weight increasing by 89 and 43 percent respectively.

To evaluate the extension of serum half-life, rats were dosed with the different peptides at 15 mg/kg, blood samples were taken at different time points, and the pharmacokinetic profiles were created. The calculated t₁/2 values are summarized in Table 6.

Variants 4 and 6 showed the most extended half-life and improved efficacy, therefore they were conjugated with different PEGs and linkers for further improvement of the longevity.

### Example 4: Binding Affinity of the Modified GLP-2 Variants to the GLP-2 Receptor

Evaluation of in vitro binding affinity of the different peptides was assessed by using Cell Based Assay (CBA) in the presence of escalating doses of the different peptides. The cells overexpress the GLP-2 receptor, and upon peptides binding they give signals which were used to calculate the EC₅₀ values. The EC₅₀ values are summarized in Table 7. The mutated GLP-2 peptides had shown lower binding affinities compared to the control GLP-2, which contains one single substitution at position 2 (GLP-2-Gly2, Teduglutide). Although the mutated variants #2-7 had shown similar results of binding affinities (EC₅₀ ranged between 13 to 36), just two variants showed significant improvement of the in vivo efficacy as was measured by the intestine weight model (Table 6). The Teduglutide peptide had shown the highest binding affinity, even compared to the GLP2 Kit control, with EC₅₀ of 5.6 nM. Despite having the lowest EC₅₀, GLP-2-Gly2 showed a moderate in vivo efficacy with ˜25% increasing of intestine weight after daily injection over 6 days (Table 1).

TABLE 7
EC₅₀ results of different modified peptides

| Variant | | EC₅₀ | Ratio to |
|---|---|---|---|
| # | Sequence | [nM] | Teduglutide |
| 2 | NH2-HG²E³GSFSDE(Nle)¹⁰(D- | 36.1 | 6.4 |
| | F)¹¹TILDN¹⁶LAARDFINWLIQTKITD-NH2 | | |
| 3 | NH2-HG²E³GSFSDE(Nle)¹⁰(D- | 14.2 | 2.5 |
| | H)¹¹TILDN¹⁶LAARDFINWLIQTKITD-NH2 | | |
| 4 | NH2-HG²E³GSFSDE(Nle)¹⁰N¹¹TILDL¹⁶LAARDFINWLIQTKI | 21.6 | 3.8 |
| | TD-NH2 | | |
| 5 | NH2-HG²E³GSFSDE(Nle)¹⁰N¹¹TILDY¹⁶LAARDFINWLIQTKI | 13.6 | 2.4 |
| | TD-NH2 | | |
| 6 | NH2-HG²E³GSFSDE(Nle)¹⁰N¹¹TILDL¹⁶LAARDFINWLIQTKI | 21.7 | 3.8 |
| | TD-COOH | | |
| 7 | NH2-HG²E³GSFSDE(Nle)¹⁰N¹¹TILDY¹⁶LAARDFINWLIQTKI | 17.2 | 3.0 |
| | TD-COOH | | |
| Control 1 | NH2-HGDGSFSDEMNTILDNLAARDFINWLIQTKITD-COOH | 5.6 | NA |
| | (GLP-2 Gly2, Teduglutide) | | |
| Control 2 | GLP2 DiscoverX Kit | 10.1 | 1.8 |

Further comparison of V4 to other GLP2 based drugs was performed by evaluating EC₅₀ (nM) as measured by in vitro binding affinity to the GLP2-receptor. V4, Apaglutide, Glepaglutide, and Teduglutide were compared in 3 independent CBA (Table 8). V4 consistently showed lower EC₅₀ values compared to Apraglutide and Glepaglutide. Teduglutide consistently showed the lowest EC₅₀ even compared to the positive control of GLP2 (DiscoverX). On average, V4, Apraglutide and Glepaglutide showed a 1.8-fold, 2.9-fold and 9.4-fold increase, respectively, in EC₅₀ compared to Teduglutide. The absolute EC₅₀ values differ between Table 7 and Table 8, which most likely was due to the change of the GLP-2R Assay Kit, containing cells from a different lot number. Despite the difference in EC₅₀ values between the two Tables, the EC₅₀ trend is quite similar: V4>Kit control >Teduglutide.

TABLE 8
EC₅₀ values of V4, Teduglutide, Apraglutide and
Glepaglutide based on GLP2-receptor CBA assays.

| | EC₅₀ (nM) | | EC₅₀ Ratio to | EC₅₀ Ratio to |
|---|---|---|---|---|
| Test Article | Average | STD | Teduglutide | Variant 4 |
| V4 | 1.0 | 0.2 | 1.8 | NA |
| Apraglutide (V1) | 1.7 | 0.1 | 2.9 | 1.6 |
| Glepaglutide (V10) | 5.4 | 1.6 | 9.4 | 5.3 |
| Teduglutide | 0.6 | 0.1 | NA | 0.6 |
| GLP2 DiscoverX Kit | 0.8 | 0.1 | 1.3 | 0.7 |

### Example 5: Construction of Conjugated GLP-2 Variant

Several combinations of PEGs (between 20 kDA and 40 kDa) and linkers (FMS, Fmoc, or MeOFmoc) were conjugated with the selected GLP-2 variants #4 and #6.

Three different PEGylated GLP-2 variants using mutated peptide variant #6 were synthesized and injected to rats to evaluate their efficacy in the intestine model. In the study, the PEGylated polypeptides of GLP-2 variant #6 consist of a heterogenous product containing a mixture of PEG attached to variant #6 via a linker at the N terminus and PEG attached to variant #6 via a linker at the lysine residue on position number thirty (Lys30).

PEG20-FMS-V6, PEG20-Fmoc-V6 and PEG40-FMS-V6 were injected once at 1 mg/kg dose on day 1, while group D was also injected twice on days 1 & 3 at 1 mg/kg. The intestine weight was performed on day 6. The results are presented in Table 9.

TABLE 9
Pharmacology results of reversible PEGylated GLP-2 Variant 6

| | | | | Increase of |
|---|---|---|---|---|
| | | | | intestine |
| | | Peptide | # of | weight over |
| Group | Material | dose mg/kg | Injections | vehicle (%) |
| (A) | PEG(20)-FMS-V6 | 1 | 1 | 11 |
| (B) | PEG(20)-Fmoc-V6 | | 1 | 10 |
| (C) | PEG(40)-FMS-V6 | | 1 | 12 |
| (D) | PEG(40)-FMS-V6 | | 2 | 45 |

The synthesis process for the PEGylated GLP-2 variant #4 polypeptides described in Table 9 is composed of two steps in which coupling of the linker is executed on GLP-2 variant peptide while it is on resin in a controlled and site-directed manner. Designing a chemical protecting groups enabling the protection of peptide active groups such as, but not limit to N-terminal, His side chain, Lys side chain, result in a site-directed linker coupling where one or several linkers (either homogeneous or heterogeneous in type) can be specifically linked to the mutated GLP-2 peptide. Following linker coupling to the peptide on resin the maleimide active group reduction can be performed, if desired, using but not limited to thiol containing molecules (e.g, Cysteine). Following peptide coupling, a cleavage from the resin and purification is performed using conventional methods known to those of skill in the art. Following peptide-linker purification, in cases were the maleimide active group of the MAL-linker-peptide was not reduced, PEGylation is performed with the purified MAL-Linker-GLP-2 variant. By utilizing this on-resin procedure, the two homogenous conjugation variants can be synthesized: PEG-Linker-(N-terminal)-GLP-2 variant, PEG-Linker-(Lys30)-GLP-2 variant (FIG. 9).

The manufacturing process of the PEGylated GLP-2 variant #4 polypeptides includes solid phase peptide synthesis (SPPS) of the GLP-2 variant peptide (stage 1), sulfonation of Fmoc linker (pre-linker) to FMS linker (stage 2—optional), coupling of the Linker to GLP-2 variant on the SPPS resin, reduction of the maleimide group (optional—in cases were PEGylation is not desired), cleavage of peptide from the resin (stage 3.1), purification of MAL-Linker-GLP-2 (or linker-peptide) variant as a key intermediate or API if PEGyaltion will not take place (stage 3.2), PEGylation and purification in cases were the MAL active group was not reduced (stage 4), salt exchanging and microfiltration (stage 5) and final lyophilization (stage 6).

In the study, the PEGylated polypeptides of GLP-2 variant #4 consist of a homogenous product containing PEG attached to variant #4 via a linker at the N terminus.

Single dose of reversible PEGylated variant 6 at 1 mg/kg did not show an increase in the intestine weight. However, two injections at 1 mg/kg showed significant increase (P<0.001).

Three different reversible PEGylated GLP-2 variants using mutated peptide #4 were injected at 2 mg/kg (peptide dose), once every 6 days for 12 days. On the 12^{th} day the intestine was weighted and compared to the control group. PEG30-FMS-V4 was injected at 0.5 mg/kg as well to evaluate the efficacy dose range. Results are summarized in Table 10.

TABLE 10
Pharmacology results of reversible PEGylated GLP-2 Variant 4

| | | Peptide | Material | Average | % CV of | Increase of |
|---|---|---|---|---|---|---|
| | | dose | dose | Intestine | Intestine weight | intestine weight |
| Group | Material | mg/kg | mg/kg^{a} | weight (gr) | between animals | over vehicle (%) |
| (A) | PEG(30)-FMS-V4 | 2 | 19 | 6.9 | 7.7 | 15 |
| (B) | PEG(30)-FMS-V4 | 0.5 | 5 | 5.2 | 9.6 | −14 |
| (C) | PEG(30)-Fmoc-V4 | 2 | 19 | 7.8 | 6.5 | 29 |
| (D) | PEG(40)-Fmoc- | 2 | 27 | 6.9 | 4.8 | 14 |
| | (GLP-2-Gly2) | | | | | |
| (E) | Vehicle | N/A | N/A | 6 | 5.4 | N/A |

^{a}The material dose was calculated based on its peptide content and purity
Dosing of reversible PEGylated GLP-2 variant 4 at 1 mg/kg, once every 6 days, for 12 days showed significant increase in intestine weight (15 and 29%, P < 0.01 or P < 0.001, respectively. In addition, the lower dose of PEG30-FMS-V4 (0.5 mg/kg) did not result with intestine increase after 12 days of treatment.

### Example 6: The Effect of Different Constructions of Conjugated Variant 4 on Intestinal Epithelium Structure

In addition to PEG30-FMS-V4 and PEG30-Fmoc-V4 that have been pharmacology evaluated by small intestinal weight evaluation (As shown in Example 5), four other reversible PEGylated variant 4 (“V4”) conjugates and two reversible linker-V4 conjugates were synthesized. All the above were injected to 6 normal SD rats/group once at 2 mg/kg (peptide dose), while on the 6^{th} day, animals were scarified, and the small intestine was weighted followed by histopathology evaluation. GLP-2 analogs can significantly increase villi height and crypts depth. Therefore, slices from three areas of the intestine (duodenum, jejunum and ilium) were stained with hematoxylin and eosin and at least 7 Crypts+Villi length/slice were measured. The averaged Villi+Crypt length of each treatment group (n=6) was compared to the vehicle group (negative control) and calculated as percentage increase over vehicle.

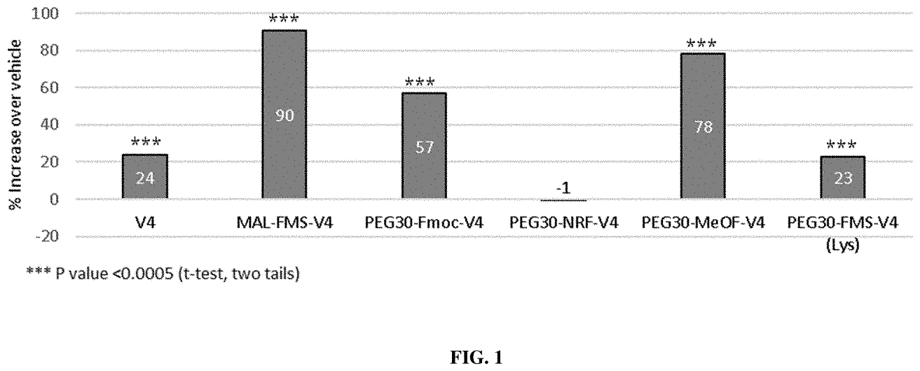

In addition to the reversible PEGylated conjugates, a non-reversible PEGylated V4 conjugate was used to assess the advantage of the reversible characteristic of the studied conjugates. Results of two different studies, in which the above V4 conjugates were compared pharmacologically are summarized in FIGS. 1 & 2 and Table 11 (Studies 15198 and 15202).

The term “V4” used throughout FIGS. 1-3 refers to GLP-2 Analog Variant #4 having the following amino acid sequence: NH2-HGEGSFSDE(Nle)NTILDLLAARDFINWLIQTKITD-NH2 (SEQ ID NO: 4).

The term “MAL-FMS-V4” used throughout FIGS. 1-3 refers to the following structure:

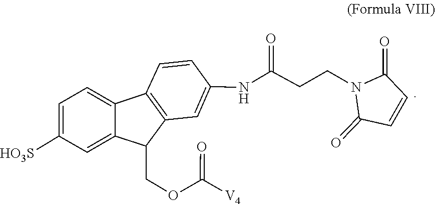

The term “PEG30-Fmoc-V4” used throughout FIGS. 1-3 refers to the following structure:

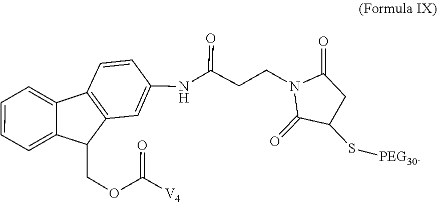

The term “PEG30-NRF-V4” used throughout FIGS. 1-3 refers to the following structure:

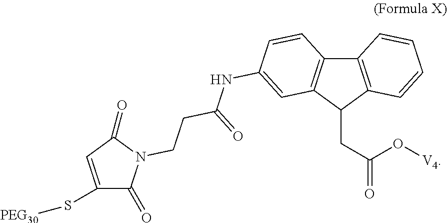

The term “PEG30-MeOF-V4” used throughout FIGS. 1-3 refers to the following structure:

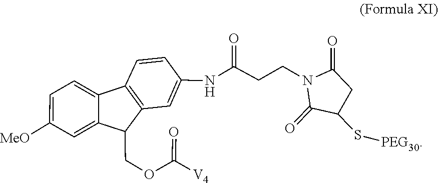

The term “PEG30-FMS-V4 (Lys)” used throughout FIGS. 1-3 refers to the following structure:

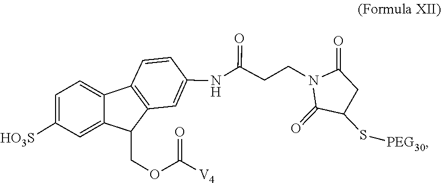

wherein the linker is attached to the GLP-2 Variant #4 at the Lysine position 30 of the GLP-2 analog.

The term “PEG30-FMS-V4” used throughout FIGS. 1-3 refers to the following structure:

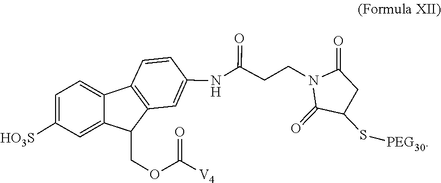

The term “PEG20MA-FMS-V4” used throughout FIGS. 1-3 refers to the following structure:

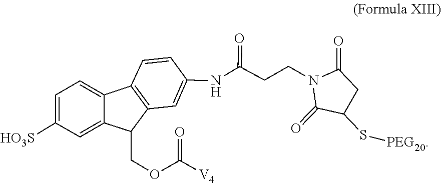

The term “PEG20MA-Fmoc-V4” used throughout FIGS. 1-3 refers to the following structure:

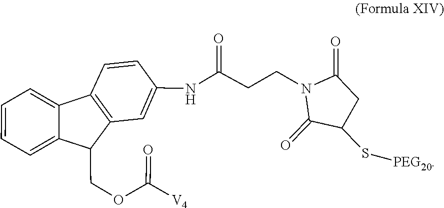

FIG. 1 shows the pharmacology effect of different V4 conjugates as measured by the percent increase of Crypts plus Villi length over vehicle.

Table 11 summarizes the pharmacology effect of different V4 conjugates as measured by the percent increase of small intestine weight over vehicle.

In this study, variant 4 linked to an FMS linker without a PEG molecule (MAL-FMS-V4) quite unexpectedly showed the most pronounce effect on the intestine epithelium, however was less effective with regards to small intestine weight. The PEG30-Fmoc-V4 and PEG30-MeOF-V4 surprisingly did not show any advantage over the MAL-FMS-V4 conjugate but did exhibit better effect on the intestine epithelium than the V4 peptide which was injected as is. However, V4 peptide which was injected as is, had the most pronounced effect on the small intestine weight. PEG30-FMS that was linked to the peptide through the lysin residue, showed low potential to affect the villi and crypts as well as small intestine weight. As was expected, the non-reversible conjugate did not cause for any increase in the intestine epithelium or weight. The full conjugate (using 30 kDa PEG and non-reversible Fmoc linker) did not possess any pharmacology effect.

TABLE 11
Pharmacology results of Variant 4 and various PEGylated GLP-2 Variant
4 conjugates as measured by small intestine weight increase

| | Avg. small intestine | Increase over |
|---|---|---|
| Test Article | weight (gr) | vehicle (%) |
| V4 | 7.0 | 43.0** |
| Vehicle 2 - 0.8% PS80 in | 5.0 | NA |
| 20 mM Na-Pi pH = 6.8 | | |
| MAL-FMS-V4 (compared | 7.0 | 21.0* |
| to Vehicle 2) | | |
| PEG30-Fmoc-V4 | 7.0 | 36.0** |
| PEG30-NRF-V4 | 4.0 | −8.0 |
| PEG30-MeOF-V4 | 7.0 | 39.0** |
| PEG30-FMS-V4 (Lys) | 6.0 | 18.0** |
| Vehicle - 20 mM Na-Pi | 5.0 | NA |
| pH = 6 | | |

*P < 0.05,
**P < 0.005

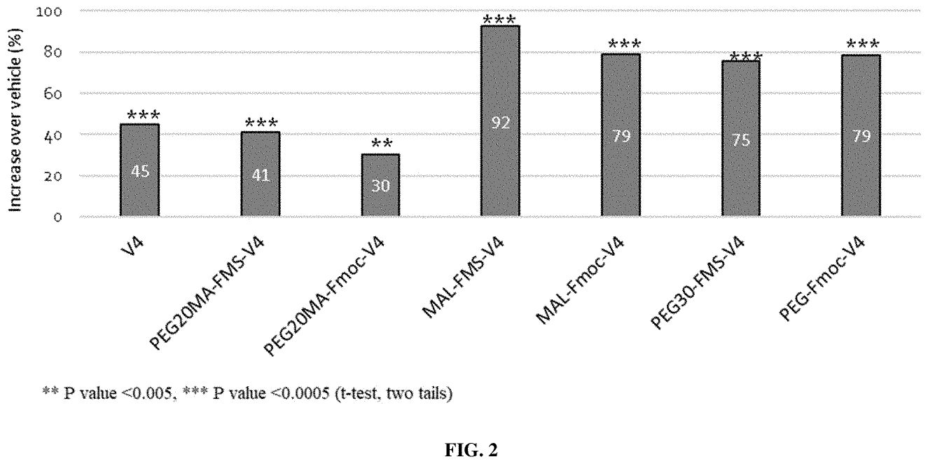

FIG. 2 shows the second pharmacology comparison of these different V4 conjugates as measured by the percent increase of Crypts plus Villi length over vehicle (Study reference 15202).

In this study, a head to head comparison between FMS and Fmoc was performed by using Multi Arm (MA) 20 kDa reversible PEGylated V4, reversible linker-V4 and the linear 30 kDa reversible PEGylated V4. There was no significate difference between the pharmacology effect on the intestine epithelium between the FMS and Fmoc conjugates. The Multi-Arm conjugates showed similar efficacy as the V4 peptide itself, while all the other four conjugates showed significant and pronounced increase in the Villi plus Crypts length.

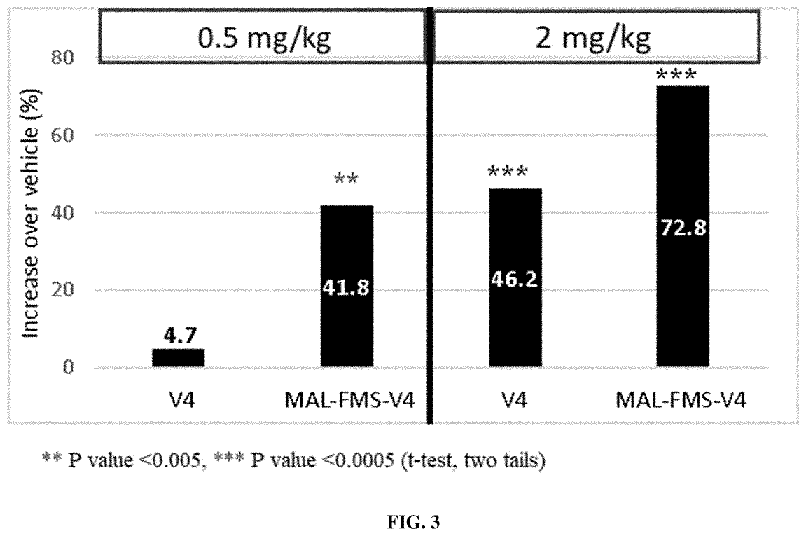

In a different study (Study reference 15201), 2 doses (0.5 and 2 mg/kg peptide dose) of V4 peptide and MAL-FMS-V4 were dosed once to 6 rat/group, while the epithelium was evaluated 6 days after injection. The results are presented in FIG. 3.

FIG. 3 shows the dose dependent pharmacology comparison between V4 peptide to reversible MAL-FMS-V4 conjugate as measured by the percent increase of Crypts+Villi length over vehicle.

Surprisingly, both V4 and MAL-FMS-V4 conjugate showed dose dependent pharmacology effect on the intestine epithelium, while the significant efficacy of the latter was consistent with previous experiments for dose of 2 mg/kg. A 0.5 mg/kg dose of the reversible linker V4 conjugate resulted with smaller increase in Crypts plus Villi length, but still significant response compares to control and V4 peptide itself.

A summary of the above studies showed that high effect on small intestine weight and villi plus crypt length can be achieved with the MAL-FMS-V4 and Variant 4 itself. Their effects were comparable or even better than the effects of PEG30-MeOF-V4, but without the need for PEGylation, hence simplifying the production of the drug. As a result, these two compounds were the focus for the rest of the development work.

### Example 7: Synthesis of Reduced Linker-Peptide

The synthesis and purification of a reduced linker-peptide is given in the following example.

Peptide is synthesized on resin containing specific protecting groups allowing the side directed linker coupling to either the peptide N-terminal, the Lys(30) side chain or combination of them both. Peptide is coupled with the Linker using the conventional methods known for those who are skilled in the art. Next, linker-coupled peptide is cleaved from the resin and further purified to result in a purified Linker-peptide. The purified linker-peptide is then lyophilized and stored till used. Dissolving the linker-peptide is performed using cysteine, or cysteamine or any thiol-containing molecule solution which allow both the dissolution and reaction of the maleimide moiety of the MAL-linker-peptide with the thiol-containing molecule. If desired, the crude reaction can be further purified and lyophilized. The following describe the synthesis and purification of cys-Linker-V4:

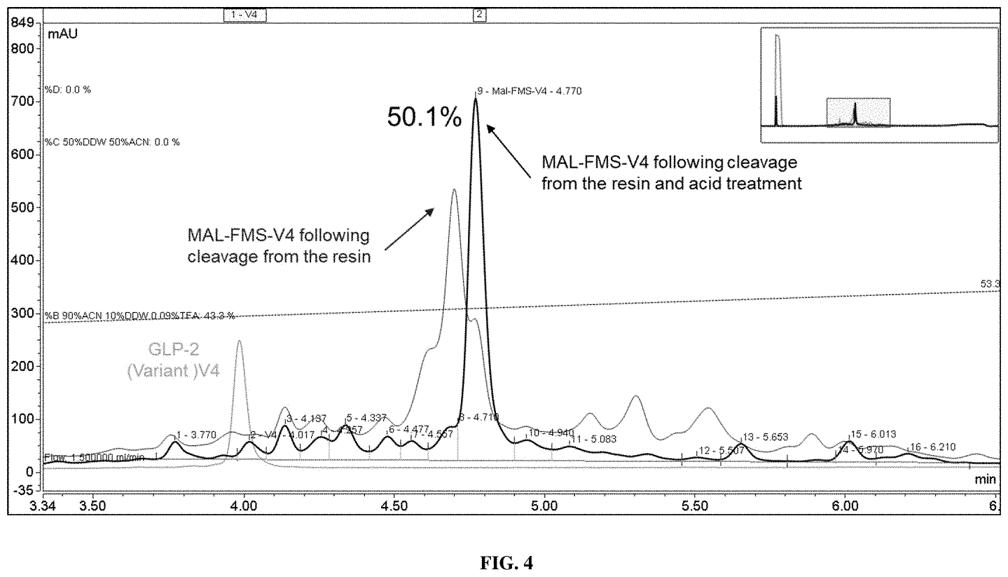

The term “Cys-FMS-V4” used throughout FIGS. 4-6 refers to the following structure:

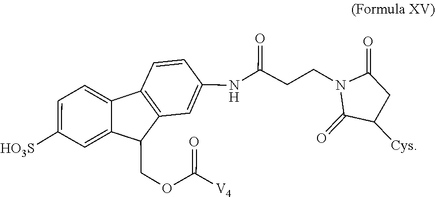

Step 1; Linker coupling to on resin V4 peptide (at a desired position either N′ terminal, Lys30, or His1 side chain.

Step 2; coupled linker-peptide cleavage from resin and purification using RP-HPLC methodology

Step 3; Purified linker-peptide lyophilization

Step 4; linker-peptide solubility and PEGylation using SH-containing PEG (e.g, PEG30-SH)

Step 5; Purification of the PEG-Linker-Peptide conjugate using RP-HPLC methodology

Step 6; Purified PEG-linker-peptide lyophilization

FIG. 4 describes RP-HPLC chromatogram showing the FMS-coupled peptide following cleavage from the resin and following acid treatment.

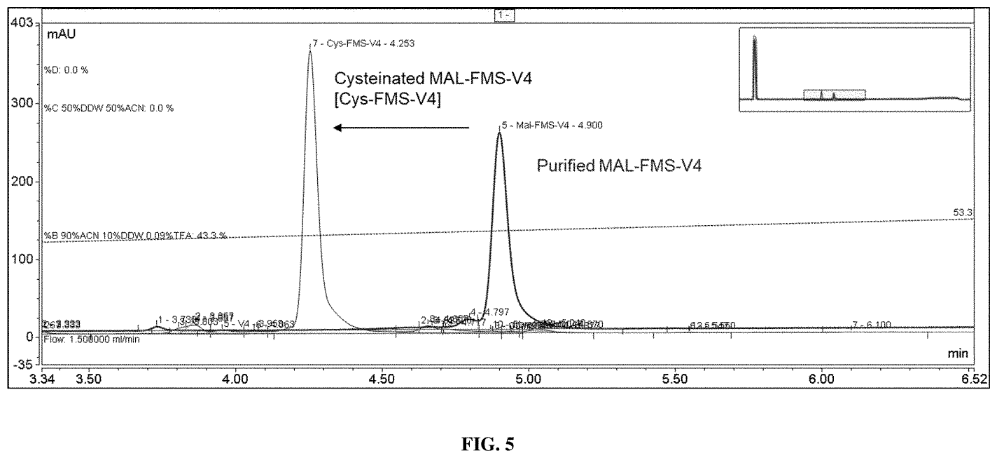

FIG. 5 describes RP-chromatogram of the purified coupled peptide and the cysteinated FMS-peptide where the cysteine covalently reacts with the maleimide group to yield Cys-FMS-V4.

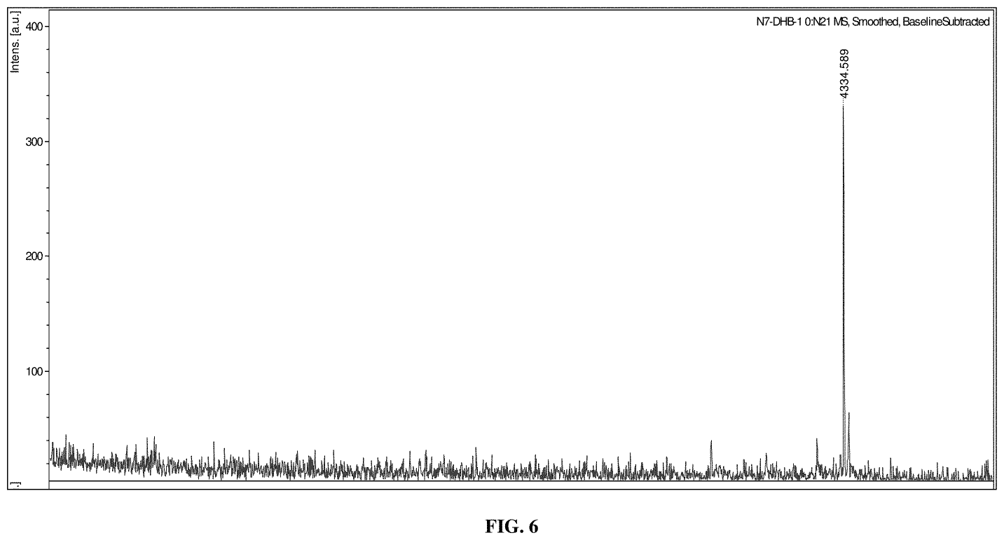

FIG. 6 describes MALDI-TOP analysis of the Cys-FMS-V4 having the expected MW of 3335 consisting from MAL-FMS-V4 MW of 4214 g/mol and the 121 g/mol gained from covalent reaction with the cysteine (121 g/mol).

In another example, the on-resin linker-coupled peptide is further reacted with thiol-containing molecule such as, but not limit to, cysteine and cysteamine resulting in the reduction of the maleimide group of the MAL-linker-peptide and the coupling of the thiol-containing molecule to the linker-peptide. Next, the thiolate-linker-peptide can be cleaved from the resin and further purified using conventional methods known for those who are skilled in the art.

The following describe all on resin synthesis cys-Linker-V4 and purification:

Step 1; Linker coupling on resin V4 peptide coupling (at a desired position either N′ terminal, Lys30, or His1 side chain.

Step 2; MAL functional group reduction using cysteine incubation while stirring of the linker-peptide on resin

Step 3; cleavage of the Cys-Linker-V4 from resin and purification using RP-HPLC methodology Step 3; Purified Cys-linker-peptide lyophilization.

### Example 8: Comparative Effect of Variant 4 and Conjugated Variant 4 to Apraglutide, Glepaglutide, and Gattex

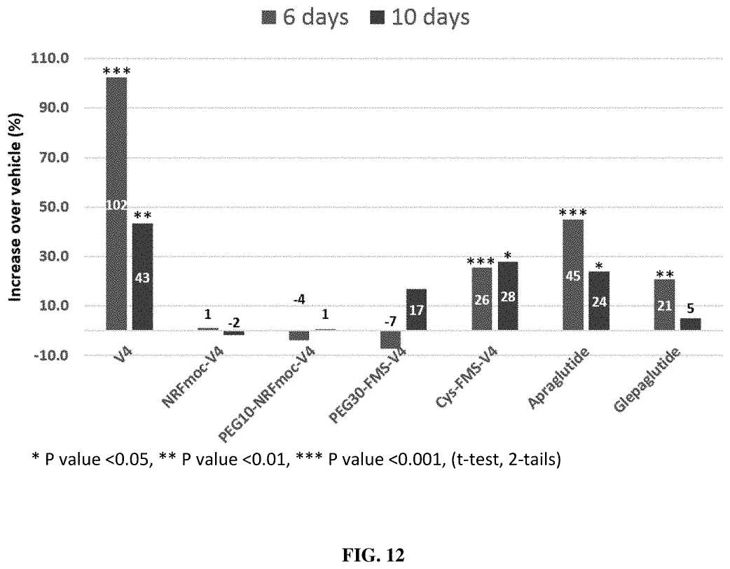

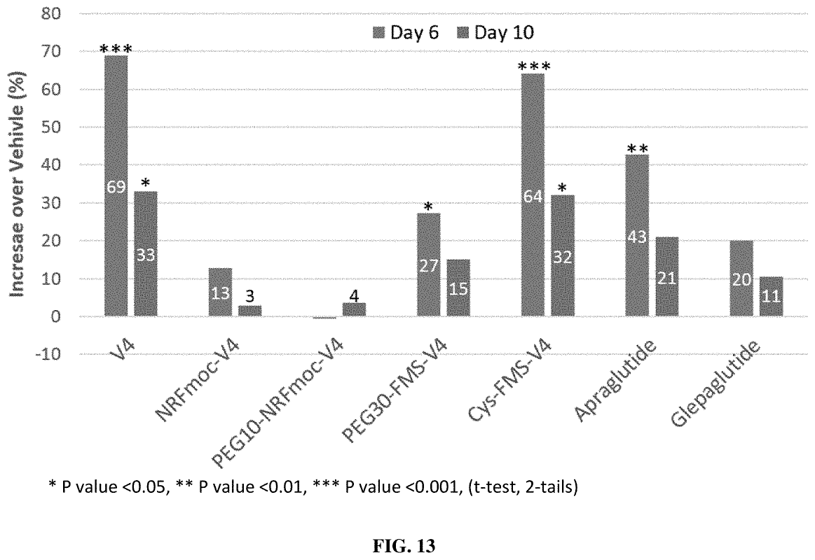

Additional studies were conducted to evaluate the efficacy of the V4 peptide and the reversible MAL-FMS-V4. The long acting effect of V4 and the conjugates were evaluated at Day 6 and Day 10 following treatment (Study reference 15203). All test groups (n=9) were injected SC with 2 mg/kg peptide content at Day 1. At Days 6 and 10, animals were sacrificed and the small intestine underwent histopathology evaluation (6 animals for Day 6, 3 animals for Day 10). FIGS. 12 and 13 show the pharmacology effect of different V4 conjugates and Apraglutide and Glepaglutide as measured by the percent increase of small intestine weight and crypts plus villi length over vehicle, respectively. In this study, for both time points, V4 showed the most favorable results with respect to small intestine weight percent increase over vehicle and comparable results to Cys-FMS-V4 with respect to crypts plus villi length percent increase over vehicle and compared to other tested conjugates. Surprisingly, V4 showed the most pronounced effect on crypts plus villi length over vehicle compared to Apraglutide and Glepaglutide. Apraglutide showed a moderate effect that was higher than Glepaglutide and the PEG30-FMS-V4 conjugate. In this experiment, V4 and Cys-FMS-V4 showed a prolonged activity 10 days following a single SC injection. Although V4, Apraglutide, and Glepaglutide are based on GLP-2, in this study they demonstrated significantly different pharmacological effects.

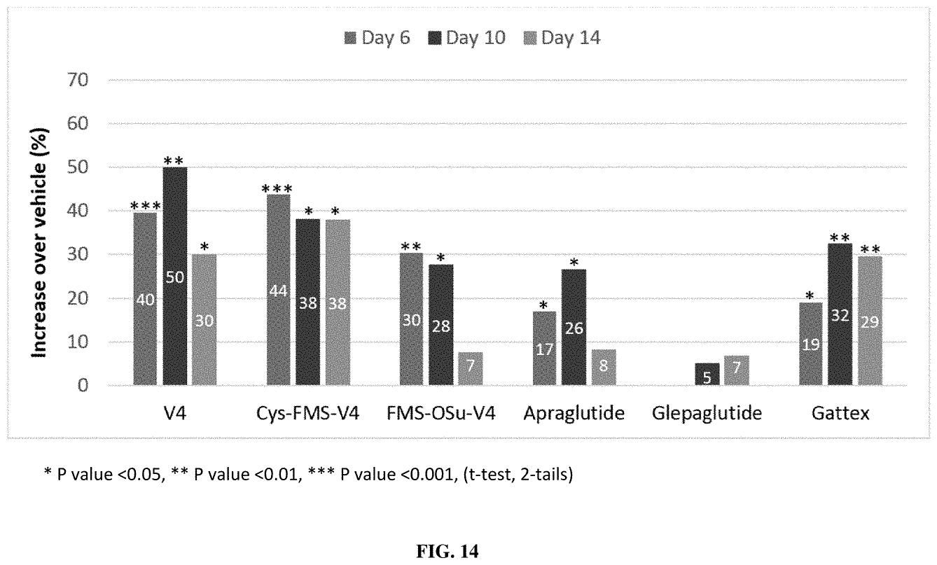

An additional study to evaluate the efficacy and the longevity of V4 and Cys-FMS-V4 was conducted (Study reference 15204) in which the animals were sacrificed on Days 6 (N=6), 10 (N=4) and 14 (N=4). This experiment measured the pharmacological response compared to the commercially approved SBS treatment drug Gattex. In addition, two new conjugates were tested by conjugating V4 to reduced OSu-linker and Cys-FMS-V4 purified from resin. The animals were injected with a single SC injection at Day 1 with 2 mg/kg peptide content of V4, Cys-FMS-V4, FMS-OSu-V4, Apraglutide and Glepaglutide while Gattex was SC injected daily at 2.5 mg/kg peptide content. FIG. 14 shows that V4 and Cys-FMS-V4 demonstrate the most prominent and prolonged effect on villi and crypts length up to 14 days from injection. On days 10 and 14, there was a significant increase (P<0.001 for V4 and P<0.01 for Cys-FMS-V4 over Apraglutide) in parameters compared to Apraglutide and Glepaglutide with comparable results to Gattex. V4 demonstrated its unexpected superiority over Apraglutide and Glepaglutide and moreover demonstrated that a single 2 mg/kg injection of V4 or Cys-FMS-V4 is sufficient to achieve comparable effect to Gattex following 13 daily injections (32.5 mg/kg accumulated dose).

### Example 9: Comparative Multi Dose Effect of Variant 4 and Cys-FMS-V4

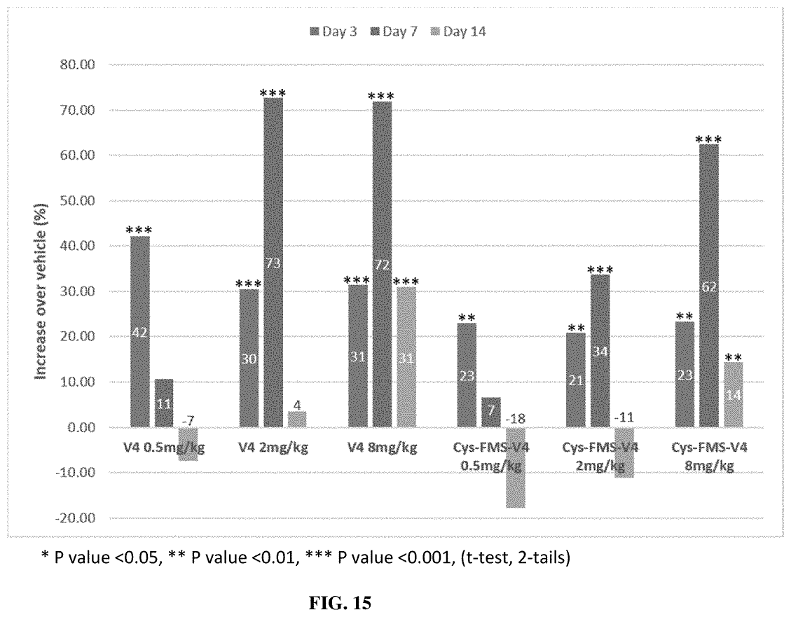

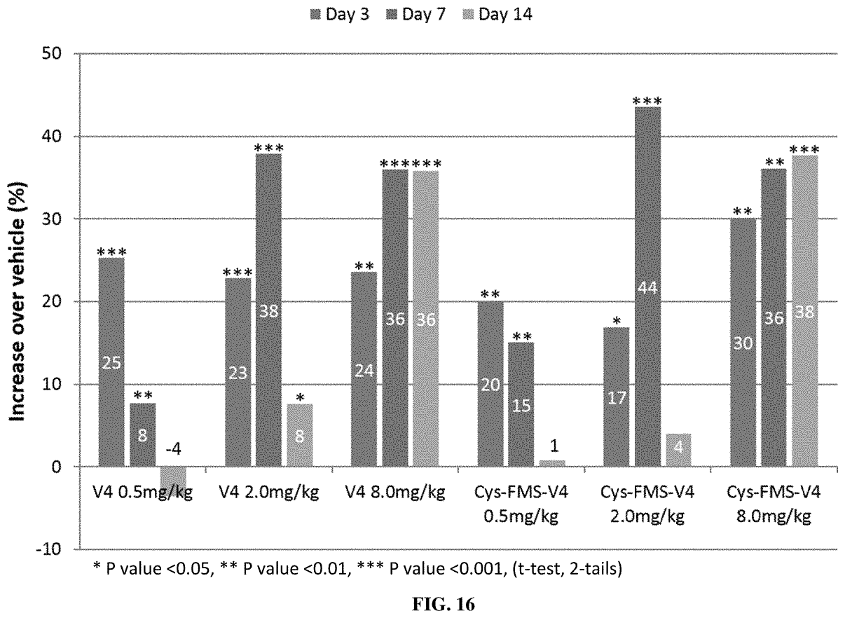

To further evaluate the pharmacology efficacy of V4 and Cys-FMS-V4, an experiment was performed to test their acute and prolonged effect on the small intestine (Study reference 15205). In addition, V4 and Cys-FMS-V4 were administrated as a single SC injection on Day 1 at 3 different doses (0.5, 2.0 or 8.0 mg/kg peptide content, 6 rats per group) to assess the dose dependence effect. The pharmacological effect on small intestine weight and villi plus crypts length was measured at Days 3, 7 and 14, and the results are presented in FIGS. 15 and 16. FIG. 15 shows that both V4 and Cys-FMS-V4 have reached maximal acute response at day 3 following treatment as demonstrated by similar small intestine weight percent increase over vehicle at all 3 dose levels. It should be noted that this acute response was higher for V4 compared to Cys-FMS-V4 at all 3 dose levels. While a low dose level of both test articles at 0.5 mg/kg was not enough to sustain the acute effect with respect to small intestine weight percent increase over vehicle, the 2.0 mg/kg dose level resulted in a more prominent and sustained effect on the small intestine weight percent increase over vehicle 7 days after treatment. At these conditions (2 mg/ml, Day 7), V4 showed a significant superiority (P<0.01) over Cys-FMS-V4. Longevity of the effect was further demonstrated with the highest dose level of 8.0 mg/kg 14 days following treatment. Although on Day 7 at 8.0 mg/kg V4 and Cys-FMS-V4 showed comparable effects, V4 demonstrated significant (P<0.001) superiority over Cys-FMS-V4 on Day 14 (FIG. 15). Interestingly, V4 showed a similar small intestine weight percent increase over vehicle compared to the 2.0 mg/kg group on Day 7 suggesting a plateau in the response was reached.

FIG. 16 shows that on Day 3, V4 and Cys-FMS-V4 induced a comparable intestinotrophic response on villi and crypt length at all three dose levels. Interestingly, the maximal level of length increase at Day 3, was similar across all doses suggesting plateau pattern. On Day 7 both V4 and Cys-FMS-V4 achieved the highest effect on length increase in the groups treated with either 2 mg/kg or 8 mg/kg. This significant effect (P<0.001) was sustained for 14 days following treatment when injecting the animals with 8.0 mg/kg peptide content. Thus, V4 and Cys-FMS-V4 have a similar pharmacological effect as measured by villi plus crypt length with an advantage for V4 on small intestine weight gain. Both compounds exhibit a dose dependent correlation following a single injection with a significant effect observed up to Day 14.

### Example 10: Pharmacokinetic Modelling of V4 and Cys-FMS-V4 Support Long Acting Potential

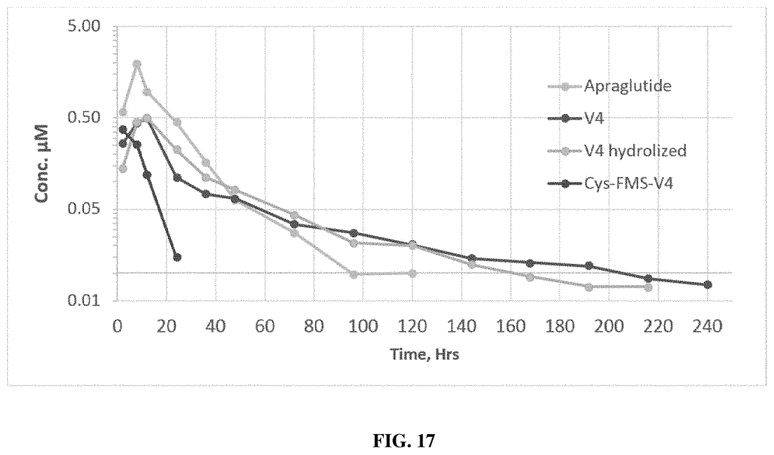

To support the pharmacodynamic effects of V4 and Cys-FMS-V4 as detailed in Example 9, a pharmacokinetic analysis was also conducted. In this study (Study reference 15204), all animals were injected SC on Day 1 with 2 mg/kg peptide content of V4, Cys-FMS-V4 and Apraglutide. Blood samples were collected at 2, 8, 12, 24, 36, 48, 72, 96, 120 hours, and Days 7, 8, 9, 10, 11, 12, 13 and 14 post-dose administration and subsequently taken to LC-MS/MS analysis to determine plasma levels of each compound. For Cys-FMS-V4, a 2-analyte method was applied to determine the plasma concentrations of both the full conjugate and the V4 that was hydrolyzed from it (V4 hydrolyzed). Plasma concentrations (μM), as measured by using the LC-MS/MS method, of V4 peptide, Cys-FMS-V4, hydrolyzed V4, and Apraglutide are presented in FIG. 17. The PK parameters are summarized in Table 12.

TABLE 12
Pharmacokinetic properties of V4, Cys-FMS-
V4, and Apraglutide following SC injection

| | | | V4 | Cys- | |
|---|---|---|---|---|---|
| Parameter | Unit | V4 | (hydrolyzed) | FMS-V4 | Apraglutide |
| t½ | h | 77 | 65.6 | 3.9 | 14.5 |
| Tmax | h | 12 | 12 | 2 | 8 |
| Cmax | μmol/L | 0.49 | 0.5 | 0.37 | 1.95 |
| AUC 0-t | μmol/L*h | 13.7 | 15.1 | 3.8 | 29.2 |
| AUC 0- | μmol/L*h | 14.6 | 15.5 | 3.8 | 29.4 |
| inf_obs | | | | | |
| Vz/F_obs | (μmol/kg)/ | 4.07 | 2.19 | 0.82 | 0.38 |
| | (μmol/L) | | | | |
| Cl/F obs | (μmol/kg)/ | 0.037 | 0.034 | 0.144 | 0.018 |
| | (μmol/L)/h | | | | |

As shown in Table 12 and FIG. 17, Apraglutide had apparent half-life (t_{1/2}) of 14.5 hours, which is comparable to the previously published PK parameters by Hargrove et al. (Hargrove D M et al. Pharmacological characterization of FE 203799, a novel long-acting peptide analog of glucagon-like peptide-2 (GLP-2). Gastroenterology 2011; 140 (Suppl. 1): S293). V4 and the hydrolyzed V4 from Cys-FMS-V4 had shown an improved longevity compared to Apraglutide, with apparent half-life of 77 and 65.6 hours. The full conjugate Cys-FMS-V4 had a short half-life of 3.9 hours, which can be explained by its rapid hydrolysis in the site of injection and in the blood. Compared to Apraglutide, V4 and the hydrolyzed V4 can be detected much later, up to Day 10 (240 h post injection). Although Apraglutide reaches a higher concentration (Cmax) it is eventually eliminated faster from the blood compared to V4 and cannot be detected after Day 6 (120 h). Apraglutide has a higher total exposure (AUC), but the overall volumes of distribution (Vz/F) of V4 and hydrolyzed V4 are much higher. This suggest that V4 is distributed more significantly to peripheral tissues to generate a sustained reservoir which potentially contributes to its long acting efficacy. This translates into a prolonged and flat PK profile that corresponds to the intestinotrophic effects observed up to Day 14 as measured by small intestine weight and crypt plus villy length percent increase over vehicle (see FIGS. 12-14).

In order to evaluate the bioavailability and PK profile of V4 and Cys-FMS-V4 following IV administration, animals were injected IV with 2 mg/kg of V4 and Cys-FMS-V4 (peptide content, Study reference 15206). Blood samples were collected at 0.5, 2, 4, 8, 12, 24, 36, 48, 72, 96- and 120-hours post-dose administration. The results are summarized in Table 13.

TABLE 13
Pharmacokinetic properties of V4 and
Cys-FMS-V4 follow IV injection

| | | | V4 (Cys- | Cys- |
|---|---|---|---|---|
| Parameter | Unit | V4 | FMS-V4) | FMS-V4 |
| t½ | h | 3.2 | 8.1 | 2.8 |
| Tmax | h | 0.5 | 2 | 0.5 |
| Cmax | μmol/L | 7.1 | 0.95 | 3.6 |
| AUC 0-t | μmol/L*h | 33.7 | 8.3 | 8.5 |
| AUC 0-inf_obs | μmol/L*h | 36.2 | 12.8 | 8.7 |
| Vz/F_obs | (μmol/kg)/ | 0.068 | 0.487 | 0.260 |
| | (μmol/L) | | | |
| Cl/F_obs | (μmol/kg)/ | 0.015 | 0.042 | 0.064 |
| | (μmol/L)/h | | | |

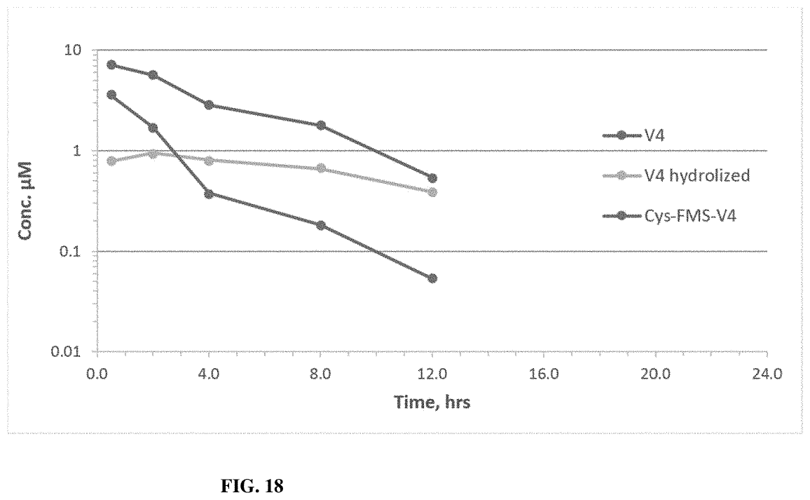

FIG. 18 shows the PK profile of V4 and Cys-FMS-V4 when administered IV at 2 mg/kg. As shown in Table 13 and FIG. 18, following IV administration, all compounds were rapidly cleared from the blood as V4, hydrolyzed V4 (V4(Cys-FMS-V4), and Cys-FMS-V4 could not be detected 24 hours post injection. V4 exhibited a short half-life (t_{1/2}) of 3.2 hours but with a greater exposure (AUC) compared to hydrolyzed V4 most likely due to rapid Cys-FMS-V4 clearance (33.7 μmol/L*h and 8.3 μmol/L*h, respectively). The conjugate Cys-FMS-V4 releases ˜35% of V4 (36.2/12.8=35, AUC ratio), and the rest is degraded with a half-life of 2.8 hrs. Hydrolyzed V4 has a longer half-life of 8.1 hours with a higher volume of distribution compared to V4 (Vz, 0.487 (μmol/kg)/(μmol/L) and 0.068 (μmol/kg)/(μmol/L), respectively) with a Cmax (0.95 μmol/L) at 2 hours post injection.

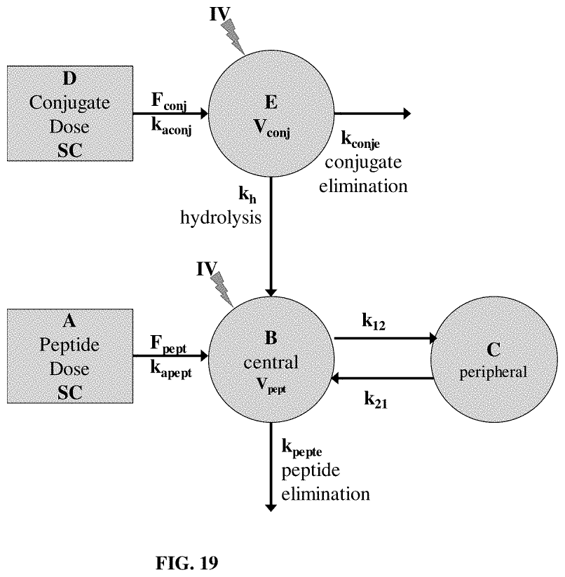

An in-depth PK modeling was performed to address the unexpected differences in half life, distribution, and pharmacological effect between V4, Cys-FMS-V4, and Apraglutide in the rat model. The analysis was performed with MONOLIX 2018R2 suite using a 2-compartmental pharmacokinetic model. The data used in this analysis was comprised from 3 independent experiments (data is presented as their average). The concentration versus time data of V4 that was formed from the Cys-FMS-V4 conjugate (the hydrolyzed peptide) was described appropriately using the values of the pharmacokinetic parameters of V4 peptide. Thus, the disposition of the hydrolyzed V4 was identical to that of the V4 peptide following the IV and SC administration. The pharmacokinetic model is presented in FIG. 19 and the results are summarized in Tables 14 and 15.

TABLE 14
Average pharmacokinetic properties of V4and Apraglutide

| | | | | Ratio |
|---|---|---|---|---|
| Parameter | Unit | V4 | Apraglutide | (V4/Apraglutide) |
| Fₚₑₚₜ | — | 0.401 | 0.74 | 0.54 |
| kₐ_{_{—}}ₚₑₚₜ | 1/hr | 0.0729 | 0.1661 | 0.44 |
| k₁₂ | 1/hr | 0.0749 | 0.0065 | 11.60 |
| k₂₁ | 1/hr | 0.0105 | 0.0068 | 1.54 |
| kₚₑₚₜₑ | 1/hr | 0.205 | 0.0855 | 2.40 |
| Vₚₑₚₜ | mL/kg | 62.7 | 156 | 0.40 |
| Vₛₛ | mL/kg | 510 | 303 | 1.68 |
| V_{beta} | mL/kg | 3212 | 2116 | 1.52 |
| CL | mL/hr/kg | 12.9 | 13.3 | 0.96 |
| t_{1/2} absorption | hr | 9.51 | 4.17 | 2.28 |
| t_{1/2} alpha | hr | 1.29 | 7.50 | 0.17 |
| t_{1/2} beta | hr | 173.2 | 110.1 | 1.57 |

TABLE 15
Average pharmacokinetic properties of Cys-FMS-V4

| Parameter | Unit | Cys-FMS-V4 |
|---|---|---|
| F_{conj} | — | 0.538 |
| kₐ_{_{—}}_{conj} | 1/hr | 0.0717 |
| k_{conje} | 1/hr | 0.207 |
| kₕ | 1/hr | 0.438 |
| V_{conj} | mL/kg | 119 |
| % to V4 | % | 67.9 |
| t_{1/2} absorption | hr | 9.67 |
| t_{1/2} other pathways | hr | 3.35 |
| t_{1/2} hydrolysis | hr | 1.58 |

The pharmacokinetics of V4 peptide is characterized by a relatively slow absorption from the SC injection site to the systemic circulation with absorption half-life of 9.51 hour (Table 14). Approximately 40% of V4 reached the systemic circulation following SC injection, indicating local degradation of ˜60% of the dose at the injection site. The initial volume of distribution of V4 is 62.7 mL/kg, which indicates a limited initial permeation into the extracellular fluid (rat plasma volume is ˜31.2 mL/kg). The distribution phase of V4 is rapid (t_{1/2 alpha}=1.29 hr) and is masked by the absorption kinetics following the SC administration. In contrast, the terminal half-life is much longer (t_{1/2 beta}=173 hours) than the absorption and initial distribution processes and resulted in a relative prolonged presence of V4 in the plasma, after IV or SC administration (FIGS. 17 and 18). This slow elimination of V4 is most likely due to substantial accumulation of V4 in extravascular locations (Vₛₛ=510 mL/kg, and V_{beta}=3212 mL/kg; ˜8 and ˜51-fold increase, respectively, as compared to the initial volume of V4 distribution).

The pharmacokinetics of Apraglutide in our studies was determined after SC administration. Therefore, to allow a direct comparison between V4 and Apraglutide, it was assumed that absolute bioavailability of Apraglutide following SC administration in rats equals 74%, as previously reported. Based on this assumption, the values of volumes of distribution and clearance (Vₚₑₚₜ, Vₛₛ, V_{beta}, and CL) were calculated for Apraglutide (Table 13). The values of the other parameters of Apraglutide in Table 14 (the rate constants and the half-lives) are not dependent on this assumption.

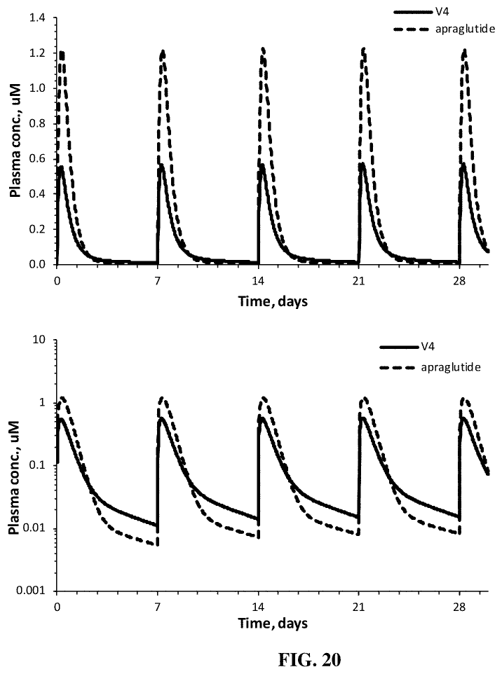

In comparison to Apraglutide, V4 bioavailability was 46% lower (Fₚₑₚₜ, 74% and 40.1%, respectively). It is characterized by approximately 2-fold slower absorption from the SC injection site to the systemic circulation (see the ratios of k_{a_pept} and t_{1/2 absorption} values in Table 14). V4 had a smaller initial volume of distribution, but tended to accumulate more extensively in the peripheral compartment, as compared to Apraglutide (See the Vₚₑₚₜ, k₁₂, k₂₁, Vₛₛ, and V_{beta}, ratios in Table 14). The clearance values of both these peptides were similar. As a result, the terminal half-life of V4 was substantially longer than that of Apraglutide (See Table 14). Visual presentation of the differences in the pharmacokinetic behavior of V4 and Apraglutide is shown in FIG. 20 (upper panel; lower panel is on a logarithmic scale) and Table 16, as simulation of the time course plasma concentrations of these compounds, based on the average values of the PK parameters in rats (simulations were not based on the assumption regarding the absolute bioavailability of Apraglutide). The simulation presents a dose regimen of 5 weeks, one single injection per week of 2 mg/kg SC. In the simulation, V4 showed a flatter PK profile with higher trough concentrations (0.57 μM, C_{ss min}), indication of its prolonged presence in the blood. The peak blood concentration (0.099 μM C_{ss max}) may provide an advantage from a safety point of view, as V4 concentrations can be maintained low within an extended therapeutic window.

TABLE 16
The trough, average, and peak steady state plasma concentrations
(μM) of the V4 and apraglutide peptides, based
on the simulations presented in FIG. 20.

| Dosage regimen | Peptide | C_{ss min} | C_{ss av} | C_{ss max} |
|---|---|---|---|---|
| 2 mg/kg, SC | V4 | 0.016 | 0.099 | 0.570 |
| once a week | Apraglutide | 0.008 | 0.176 | 1.224 |

Administration of V4 in the form of Cys-FMS-V4 conjugate led to more efficient absorption of the conjugate to the central circulation (53.8% vs. 40.1%), with similar kinetics (9.67 hours vs. 9.51 hours absorption half-life), as compared to those of the V4 peptide (see Tables 14 and 15). The volume of distribution of the Cys-FMS-V4 conjugate was lower than the steady state volume of distribution of the V4 peptide (119 mL/kg vs. 510 mL/kg). Approximately ⅔ of the absorbed Cys-FMS-V4 is hydrolyzed to V4. Thus, approximately 36.5% of Cys-FMS-V4 following SC administration was converted to V4 (53.8% SC bioavailability, 67.9% hydrolyzed to V4). The kinetics of Cys-FMS-V4 absorption and hydrolysis to V4 was rather rapid (the t_{1/2} values of 9.67 hours and 1.58 hours, respectively), and the elimination kinetics of V4 peptide (t_{1/2} beta) governed the time course of V4 plasma concentrations after SC Cys-FMS-V4 administration.

Although V4 and Apraglutide differ in only 2 amino acids (V4-E³ and N¹¹, Apraglutide-D³ and D-F¹¹), their pharmacokinetic parameters were markedly and unexpectedly different. Based on the modeling, they have unequivocal bioavailability, absorption, half-life, and distribution values that lead to a distinct pharmacological effect. V4 was slowly absorbed into the blood and extensively accumulated in peripheral tissues, thus presenting a sustained release profile as the peptide was slowly reabsorbed into the main compartment, explaining its long-term pharmacodynamics superiority over Apraglutide.

What is claimed is:

1. A compound of the formula:
L-GLP-2
wherein, L is an optional linker group; and
GLP-2 is a GLP-2 analog or variant having one or more specific amino acid mutations as compared to wild type GLP-2.

2. The compound of claim 1, wherein said linker group is 2-methoxy-9-fluorenylmethoxycarbonyl (MeOFmoc), 2,5-dioxopyrrolidin-1-yl-3-(2-(3-(2,5-dioxo-2,5-dihydro-1H-pyrrol-1-yl)propanamido)-9H-fluoren-9-yl)propanoate (“NRFmoc”), 9-fluorenylmethoxycarbonyl (Fmoc), MAL-Fmoc, Fmoc-Osu, 2-sulfo-9-fluorenylmethoxycarbonyl (FMS), MAL-FMS, or FMS-Osu.

3. The compound of any one of the preceding claims, wherein said compound is selected from one of the following formulas:

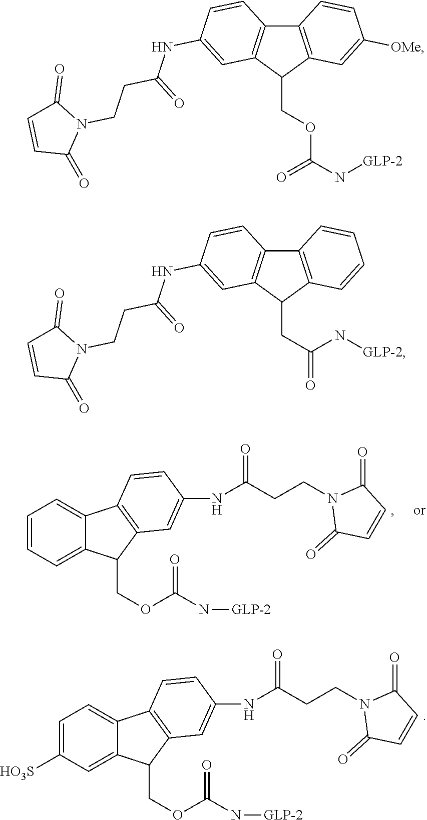

4. The compound of any one claims 1-2, wherein said compound is selected from one of the following:

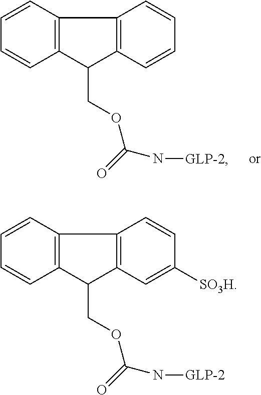

5. The compound of any one of claims 2-3, wherein the linker containing a maleimide group is further reacted with a thiol-containing molecule

6. The compound of claim 5, wherein said thiol-containing molecule is cysteine or cysteamine.

7. The compound of any one of claims 5-6, wherein said reacting results in the reduction of the MAL-linker-GLP-2 such as maleimide hydrogenation, and/or the coupling of the thiol-containing molecule to the linker-GLP-2.

8. The compound of any one of claims 1-3 and 5-7, wherein the formula further comprises:
X-L-GLP-2
wherein, X is selected from a polymeric compound.

9. The compound of claim 8, wherein X a polyethylene glycol polymer (“PEG”).

10. The compound of claim 9, wherein said PEG is PEG2, PEG10, PEG20, PEG30, PEG40, or PEG60.

11. The compound of claim 9, wherein said PEG has a molecular weight in the range of 2,000 to 50,000 Da

12. The compound of any one of the preceding claims, wherein said GLP-2 analog or variant has an amino acid sequence according the formula:
R1-His1-X2-X3-Gly4-Ser5-Phe6-Ser7-Asp8-Glu9-X10-
X11-Thr12-Ile13-Leu14-Asp15-X16-Leu17-Ala18-
Ala19-Arg20-Asp21-Phe22-Ile23-Asn24-Trp25-Leu26-
Ile27-Gln28-Thr29-Lys30-Ile31-Thr32-Asp33-R2,
wherein R1 may be OH, COOH, NH2, CONH2, or CONHNH2;
X2 may be Ala or Gly;
X3 may be Asp or Glu;
X10 may be Met or Nle;
X11 may be Asn, D-Phe, or D-His;
X16 may be Asn, Leu, or Tyr; and
R2 may be OH, COOH, NH₂, CONH₂, or CONHNH₂.

13. The composition of claim 12, wherein X2 is Gly, X3 is Glu, X10 is Nle, X16 is Leu and R2 is NH₂ or CONH₂.

14. The compound of claim 12, wherein said GLP-2 analog or variant has an amino acid sequence according to any one of SEQ ID NO: 1 through SEQ ID NO: 8.

15. The compound of any one of claims 1-11, wherein said GLP-2 analog or variant has an amino acid sequence according to any one of SEQ ID NO: 9 through SEQ ID NO: 16.

16. The compound of claim 15, wherein amino or carboxy terminus of said GLP-2 analog or variant may be OH, COOH, NH₂, CONH₂, or CONHNH₂.

17. A compound of the formula: 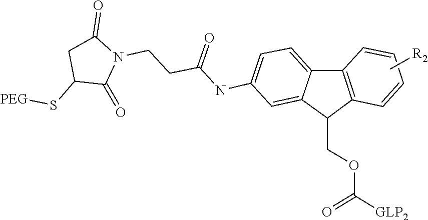
wherein
PEG is a polyethylene glycol polymer;
R2 is H, O—CH₃, or SO₃H; and
GLP2 is a GLP2 analog or variant having one or more specific amino acid mutations as compared to wild type GLP-2.

18. The compound according to claim 17, wherein said GLP-2 analog or variant has an amino acid sequence according the formula:
R1-His1-X2-X3-Gly4-Ser5-Phe6-Ser7-Asp8-Glu9-X10-
X11-Thr12-Ile13-Leu14-Asp15-X16-Leu17-Ala18-
Ala19-Arg20-Asp21-Phe22-Ile23-Asn24-Trp25-Leu26-
Ile27-Gln28-Thr29-Lys30-Ile31-Thr32-Asp33-R2,
wherein R1 may be OH, COOH, NH₂, CONH₂, or CONHNH₂;
X2 may be Ala or Gly;
X3 may be Asp or Glu;
X10 may be Met or Nle;
X11 may be Asn, D-Phe, or D-His;
X16 may be Asn, Leu, or Tyr; and
R2 may be OH, COOH, NH₂, CONH₂, or CONHNH₂.

19. The composition of claim 18, wherein X2 is Gly, X3 is Glu, X10 is Nle, X16 is Leu and R2 is NH₂ or CONH₂.

20. The compound of claim 18, wherein said GLP-2 analog or variant has an amino acid sequence according to any one of SEQ ID NO: 1 through SEQ ID NO: 8.

21. The compound of claim 17, wherein said GLP-2 analog or variant has an amino acid sequence according to any one of SEQ ID NO: 9 through SEQ ID NO: 16.

22. The compound of claim 21, wherein amino or carboxy terminus of said GLP-2 analog or variant may be OH, COOH, NH₂, CONH₂, or CONHNH₂.

23. The compound of any one of claims 17-22, wherein said GLP-2 analog is linked via an amino group of said GLP-2 analog.

24. The compound of any one of claims 17-23, wherein the linker containing a maleimide group is further reacted with a thiol-containing molecule.

25. The compound of claim 24, wherein said thiol-containing molecule is cysteine or cysteamine.

26. The compound of any one of claims 24-25, wherein said reacting results in the reduction of the MAL-linker-GLP-2 such as maleimide hydrogenation, and/or the coupling of the thiol-containing molecule to the linker-GLP-2.

27. The compound of any one of claims 24-26, wherein said PEG is a PEG polymer linked to a thiol group on said PEG polymer having the formula CH3-(O—CH2-CH2)n-S— wherein n is 5, 30, 40, or 60.

28. The compound of any one of claims 17-26, wherein said PEG is PEG2, PEG5, PEG10, PEG20, PEG30, PEG40, or PEG60.

29. The compound of any one of claims 17-26, wherein said PEG has a molecular weight in the range of 2,000 to 50,000 Da

30. The compound of any one of claims 17-29, wherein said GLP-2 analog has an extended biological half-life compared to a non-conjugated GLP-2 analog.

31. The compound of any one of claims 17-30, wherein said PEG is linear or branched.

32. A composition comprising a GLP-2 analog having an amino acid sequence according the formula:
R1-His1-X2-X3-Gly4-Ser5-Phe6-Ser7-Asp8-Glu9-X10-
X11-Thr12-Ile13-Leu14-Asp15-X16-Leu17-Ala18-
Ala19-Arg20-Asp21-Phe22-Ile23-Asn24-Trp25-Leu26-
Ile27-Gln28-Thr29-Lys30-Ile31-Thr32-Asp33-R2,
wherein
R1 may be OH, COOH, NH₂, CONH₂, or CONHNH₂
X2 may be Ala or Gly;
X3 may be Asp or Glu;
X10 may be Met or Nle;
X11 may be Asn, D-Phe, or D-His;
X16 may be Asn, Leu, or Tyr; and
R2 may be OH, COOH, NH₂, CONH₂, or CONHNH₂.

33. The composition of claim 32, wherein at least one of said X2, X3, X10, X11, and X16 is other than a wild type GLP-2 residue.

34. The composition of claim 32, wherein said GLP-2 analog incorporates at least one amino acid substitution at the following positions: X2, X3, X10, X11, and X16.

35. The composition of any one of claims 32-34, wherein R1 is NH₂.

36. The composition of any one of claims 32-35, wherein R2 is NH₂, CONH₂, or CONHNH₂.

37. The composition of any one of claims 32-36, wherein R2 is COOH.

38. The composition of claim 32, wherein X2 is Gly, X3 is Glu, X10 is Nle, X11 is D-Phe and R2 is NH₂ or CONH₂.

39. The composition of claim 32, wherein the amino acid sequence of said GLP-2 analog consists of SEQ ID NO: 2.

40. The composition of claim 32, wherein X2 is Gly, X3 is Glu, X10 is Nle, X11 is D-His, and R2 is NH₂ or CONH₂.

41. The composition of claim 32, wherein the amino acid sequence of said GLP-2 analog consists of SEQ ID NO: 3.

42. The composition of claim 32, wherein X2 is Gly, X3 is Glu, X10 is Nle, X16 is Leu and R2 is NH₂ or CONH₂.

43. The composition of claim 32, wherein the amino acid sequence of said GLP-2 analog consists of SEQ ID NO: 4.

44. The composition of claim 32, wherein X2 is Gly, X3 is Glu, X10 is Nle, X16 is Tyr and R2 is NH₂ or CONH₂.

45. The composition of claim 32, wherein the amino acid sequence of said GLP-2 analog consists of SEQ ID NO: 5.

46. The composition of claim 32, wherein X2 is Gly, X3 is Glu, X10 is Nle, and X16 is Leu.

47. The composition of claim 32, wherein the amino acid sequence of said GLP-2 analog consists of SEQ ID NO: 6.

48. The composition of claim 32, wherein X2 is Gly, X3 is Glu, X10 is Nle, and X16 is Tyr.

49. The composition of claim 32, wherein the amino acid sequence of said GLP-2 analog consists of SEQ ID NO: 7.

50. The composition of any one of claims 32-49, wherein 9-fluorenylmethoxycarbonyl (Fmoc), MAL-Fmoc, 2-sulfo-9-fluorenylmethoxycarbonyl (FMS), MAL-FMS, 2-methoxy-9-fluorenylmethoxycarbonyl (MeOFmoc), or NRFmoc is attached to one or more amino acid positions of the GLP-2 analog.

51. The composition of claim 50, wherein Fmoc, MAL-FMoc, FMS, MAL-FMS, MeOFmoc, or NRFmoc is attached at the N terminus of said GLP-2 analog.

52. The composition of claim 50, wherein the Fmoc, MAL-Fmoc, FMS, MAL-FMS, MeOFmoc, or NRFmoc is attached at the lysine residue on position number thirty (Lys30) of said GLP-2 analog.

53. A heterologous mixture of the composition of claim 51 and the composition of claim 52.

54. The composition of any one of claims 50-53, wherein polyethylene glycol (PEG) is attached to said GLP-2 analog via said Fmoc, MAL-Fmoc, FMS, MAL-FMS, MeOFmoc, or NRFmoc linker.

55. The composition of claim 54, wherein said PEG is PEG20, PEG30, PEG40 or PEG60.

56. The compound of claim 54, wherein said PEG has a molecular weight in the range of 2,000 to 50,000 Da

57. The composition of any one of claims 54-56, where PEG is branch and consisting of the formula of (PEG)m-R—SH.

58. The composition of claim 57, wherein m is 2 or 4.

59. The composition of any one of claims 54-58, wherein the Fmoc, MAL-Fmoc, FMS, MAL-FMS, MeOFmoc, or NRFmoc linker is attached at the N terminus of said GLP-2 analog.

60. The composition of any one of claims 54-58, wherein the Fmoc, MAL-Fmoc, FMS, MAL-FMS, MeOFmoc, or NRFmoc linker is attached at the lysine residue on position number thirty (Lys30) of said GLP-2 analog.

61. A heterologous mixture of the composition of claim 59 and the composition of claim 60.

62. A pharmaceutical composition comprising the compound of any one of claims 1-31 or the composition of any one of claims 32-61, or a salt or derivative thereof, in a mixture with a carrier.

63. A method for the treatment of a bowel disease, small bowel syndrome, inflammatory bowel syndrome, colitis including collagen colitis, radiation colitis, ulcerative colitis chronic radiation enteritis, non-tropical (gluten intolerance) and tropical sprue, Coeliac disease (gluten sensitive enteropathy), damaged tissue after vascular obstruction or trauma, diarrhea e.g. tourist diarrhea and post-infective diarrhea, chronic bowel dysfunction, dehydration, bacteremia, sepsis, anorexia nervosa, damaged tissue after chemotherapy e.g. chemotherapy-induced intestinal mucositis, premature infants incl. intestinal failure in premature infants, preborn infants incl. intestinal failure in preborn infants, schleroderma, gastritis including atrophic gastritis, postantrectomy atrophic gastritis and Helicobacter pylori gastritis, pancreatitis, general septic shock ulcers, enteritis, cul-de-sac, lymphatic obstruction, vascular disease and graft-versus-host, healing after surgical procedures, post radiation atrophy and chemotherapy, weight loss in Parkinson's Disease, intestinal adaptation after surgical procedure, parenteral nutrition-induced mucosal atrophy, e.g. total parenteral nutrition (TPN)-induced mucosal atrophy, and bone-related disorders including osteoporosis, hypercalcemia of malignancy, osteopenia due to bone metastases, periodontal disease, hyperparathyroidism, periarticular erosions in rheumatoid arthritis, Paget's disease, osteodystrophy, myositis ossificans, Bechterew's disease, malignant hypercalcemia, osteolytic lesions produced by bone metastasis, bone loss due to immobilization, bone loss due to sex steroid hormone deficiency, bone abnormalities due to steroid hormone treatment, bone abnormalities caused by cancer therapeutics, osteomalacia, Bechet's disease, osteomalacia, hyperostosis, osteopetrosis, metastatic bone disease, immobilization-induced osteopenia, or glucocorticoid-induced osteoporosis, the method comprising administering a therapeutically or prophylactically effective amount of the compound of any one of claims 1-31 or the composition of any one of claims 32-61 to the patient.

64. A method for the treatment of acid-induced intestinal injury, arginine deficiency, autoimmune diseases, bacterial peritonitis, bowel ischemia, bowel trauma, burn-induced intestinal damage, catabolic illness, celiac disease, chemotherapy-associated bacteremia, chemotherapy-induced enteritis, decreased gastrointestinal motility, diabetes, diarrheal diseases, fat malabsorption, febrile neutropenia, food allergies, gastric ulcers, gastrointestinal barrier disorders, gastrointestinal injury, hypoglycemia, idiopathic hypospermia, inflammatory bowel disease, intestinal failure, intestinal insufficiency, irritable bowel syndrome, ischemia, malnutrition, mesenteric ischemia, mucositis, necrotizing enterocolitis, necrotizing pancreatitis, neonatal feeding intolerance, neonatal nutritional insufficiency, NSAID-induced gastrointestinal damage, nutritional insufficiency, obesity, pouchitis, radiation-induced enteritis, radiation-induced injury to the intestines, steatorrhea, stroke, or total parenteral nutrition damage to gastrointestinal tract, the method comprising administering a therapeutically or prophylactically effective amount of the compound of any one of claims 1-31 or the composition of any one of claims 32-61 to the patient.

65. A method for increasing the crypts plus villi depth and length in a patient, the method comprising administering a therapeutically or prophylactically effective amount of the compound of any one of claims 1-31 or the composition of any one of claims 32-61 to the patient.
